(19) United States
(12) Patent Application Publication
(10) Pub. No.: US 2025/0122562 A1
(43) Pub. Date: Apr. 17, 2025

(54) PROXIMITY DETECTION OF BIOMOLECULE INTERACTIONS

(71) Applicant: Singular Genomics Systems, Inc., San Diego, CA (US)

(72) Inventor: Eli N. Glezer, Del Mar, CA (US)

(21) Appl. No.: 18/915,941

(22) Filed: Oct. 15, 2024

Related U.S. Application Data
(60) Provisional application No. 63/591,050, filed on Oct. 17, 2023.

(51) Int. Cl. C12Q 1/6874; C12Q 1/6804; C12Q 1/6841; C12Q 1/6855

(52) U.S. Cl. CPC C12Q 1/6874; C12Q 1/6804; C12Q 1/6841; C12Q 1/6855

(57) ABSTRACT

Disclosed herein, inter alia, are compositions and methods for detection of biomolecules and biomolecular interactions.

### CROSS-REFERENCE TO RELATED APPLICATIONS

This application claims the benefit of U.S. Provisional Application No. 63/591,050, filed Oct. 17, 2023, which is incorporated herein by reference in its entirety and for all purposes.

### BACKGROUND

The study of proteins is emerging as a new frontier for understanding real-time human biology. Protein biomarker discovery enables identification of signatures with pathophysiological importance, bridging the gap between genomes and phenotypes. Data including proteomic information will have a profound impact on improving future healthcare, particularly with respect to precision medicine, but progress has been hampered by the lack of technologies that can provide reliable specificity, high throughput, good precision, and high sensitivity. Expanding the knowledge of cellular protein interaction networks is vital to improve our understanding of several types of diseases, including cancer. Improved methods to study these interaction networks, especially in clinical settings, is therefore of great importance both for increasing the knowledge of the underlying disease mechanics, and also for finding new biomarkers for improved disease diagnostics and treatment response prediction. Disclosed herein, inter alia, are solutions to these and other problems in the art.

### BRIEF SUMMARY

In a first aspect is provided a method of forming a circular oligonucleotide. In embodiments, the method includes: (i) contacting a protein-probe complex with a coupling oligonucleotide, wherein the protein-probe complex includes a protein bound to a first probe and a second probe, the first probe includes a first specific binding molecule (e.g., an antibody) attached to a first oligonucleotide tag including a first barcode sequence, and the second probe includes a second specific binding molecule (e.g., an antibody) attached to a second oligonucleotide tag including a second barcode sequence, and hybridizing a first sequence of the coupling oligonucleotide to the first oligonucleotide tag and hybridizing a second sequence of the coupling oligonucleotide to the second oligonucleotide tag, thereby forming a linked complex including the protein-probe complex and the coupling oligonucleotide; (ii) contacting the linked complex with a bridge oligonucleotide and hybridizing a first sequence of the bridge oligonucleotide to the first oligonucleotide tag and hybridizing a second sequence of the bridge oligonucleotide to the second oligonucleotide tag, thereby forming a linked-bridge complex; and (iii) extending the second sequence of the coupling oligonucleotide with a polymerase to form a complement of the second barcode sequence; extending the second sequence of the bridge oligonucleotide with a polymerase to form a complement of the first barcode sequence; and ligating the complement of the second barcode sequence to the first sequence of the bridge oligonucleotide and ligating the complement of the first barcode sequence to form the circular oligonucleotide. In embodiments, the first oligonucleotide tag is the first oligonucleotide described herein. In embodiments, the second oligonucleotide tag is the second oligonucleotide described herein.

In embodiments, the method includes: (i) binding a first probe to a first protein and binding a second probe to a second protein, wherein the first probe includes a specific binding reagent attached to a first oligonucleotide including a first barcode sequence, and the second probe includes a specific binding reagent attached to a second oligonucleotide including a second barcode sequence, thereby forming a protein-probe complex; (ii) contacting the protein-probe complex with a coupling oligonucleotide and hybridizing a first sequence of the coupling oligonucleotide to the first oligonucleotide and hybridizing a second sequence of the coupling oligonucleotide to the second oligonucleotide, thereby forming a linked complex including the protein-probe complex and the coupling oligonucleotide; (iii) contacting the linked complex with a bridge oligonucleotide and hybridizing a first sequence of the bridge oligonucleotide to the first oligonucleotide and hybridizing a second sequence of the bridge oligonucleotide to the second oligonucleotide, thereby forming a linked-bridge complex; and (iv) extending the second sequence of the coupling oligonucleotide with a polymerase to form a complement of the second barcode sequence; extending the second sequence of the bridge oligonucleotide with a polymerase to form a complement of the first barcode sequence; and ligating the complement of the second barcode sequence to the first sequence of the bridge oligonucleotide and ligating the complement of the first barcode sequence to form the circular oligonucleotide.

In embodiments, the method further includes amplifying the circular oligonucleotide to form amplification products. In embodiments, the method includes detecting the circular oligonucleotide, or the amplification products. In embodiments, the method includes sequencing the circular oligonucleotide and/or the amplification products. In embodiments, the method includes sequencing the barcode sequence (e.g., the first and second barcode sequences).

### BRIEF DESCRIPTION OF THE DRAWINGS

FIGS. 1A-1E. Illustrations of the components useful for the methods described herein. FIG. 1A shows a universal padlock probe (PLP) oligonucleotide (also referred to as a “coupling oligonucleotide), including a 5′ arm region (A′), a PLP linker region (1), and a 3′ arm region (D′). The space between the 5′ and 3′ arm regions, prior to circularization, is referred to here as a “gap”. FIG. 1B provides a universal bridge oligonucleotide including a 3′ tag binding sequence (B′), a bridge linker region (2), and a 5′ tag binding sequence (C′). FIG. 1C provides a set of two barcoded probes. The first probe (3) includes a first specific binding molecule (4; e.g., an antibody), attached to a first oligonucleotide tag (5). The first oligonucleotide tag includes, from 5′ to 3′, a first coupling sequence (A) which is complementary to the 5′ arm region (A′) of the PLP illustrated in FIG. 1A, a first barcode sequence (UMI-1, also referred to as a first unique molecular identifying sequence), and a first bridge binding sequence (B) which is complementary to the 3′ tag binding sequence (B′) of the universal bridge oligonucleotide illustrated in FIG. 1B. The second probe (6) includes a second specific binding molecule (7; e.g., an antibody), attached to a second oligonucleotide tag (8). The second oligonucleotide tag includes, from 5′ to 3′, a second bridge binding sequence (C) which is complementary to the 5′ tag binding sequence (C′) of the universal bridge oligonucleotide illustrated in FIG. 1B, a second barcode sequence (UMI-2, also referred to as a second unique molecular identifying sequence), and a second coupling sequence (D) which is complementary to the 3′ arm region (D′) of the PLP illustrated in FIG. 1A. The 3′ ends of the oligonucleotide tags may include modifications (e.g., dideoxynucleotide or a reversibly-terminated nucleotide) to prevent 3′ extension and potential displacement. When applied to a biological sample (e.g., a cell or tissue), the probes bind to their respective targets, either different proteins (FIG. 1D) or different regions on the same protein (FIG. 1E), thus forming protein-probe complexes (e.g., 9 as illustrated in FIG. 1D). FIGS. 1D and 1E both illustrate how the Universal PLP serves here as a “coupling oligonucleotide” in that it anneals to the two different oligonucleotide tags due to their close proximity to each other. After the annealing of the universal bridge oligo, and the PLP, to the different oligonucleotide tags, as is illustrated in FIGS. 1D (10) and 1E, the 3′ ends of both the PLP and the Universal Bridge oligos may be extended to generate complements of the different barcode sequences, and a ligase joins the adjacent ends to form a closed circular polynucleotide, as depicted in FIG. 3. The circular polynucleotide may be amplified and detected.

FIGS. 2A-2E. Illustrations of the components useful for the methods described herein. FIG. 2A shows a universal PLP oligonucleotide, including a 5′ arm region (D′), a PLP linker region, and a 3′ arm region (A′). FIG. 2B provides a universal bridge oligonucleotide including a 3′ tag binding sequence (C′), a bridge linker region, and a 5′ tag binding sequence (B′). FIG. 2C provides a set of two barcoded probes. The first probe includes a first specific binding molecule attached to a first oligonucleotide tag (17). The first oligonucleotide tag includes, from 5′ to 3′, a first bridge binding sequence (B) which is complementary to the 5′ tag binding sequence (B′) of the universal bridge oligonucleotide illustrated in FIG. 2B, a first barcode sequence (UMI-1), and a first coupling sequence (A) which is complementary to the 3′ arm region (A′) of the PLP illustrated in FIG. 2A. The second probe includes a second specific binding molecule attached to a second oligonucleotide tag (18). The second oligonucleotide tag includes, from 5′ to 3′, a second coupling sequence (D) which is complementary to the 5′ arm region (D′) of the PLP illustrated in FIG. 2A, a second barcode sequence (UMI-2), and a second bridge binding sequence (C) which is complementary to the 3′ tag binding sequence (C′) of the universal bridge oligonucleotide illustrated in FIG. 2B. The 3′ ends of the oligonucleotide tags may be attached to their respective specific binding molecules at their 3′ end as a means to prevent extension. When applied to a biological sample (e.g., a cell or tissue), the probes bind to their respective targets, either different proteins (FIG. 2D) or different regions on the same protein (FIG. 2E), thus forming protein-probe complexes. After the annealing of the universal bridge oligo, and the PLP, to the different oligonucleotide tags, the 3′ ends of both the PLP and the Universal Bridge oligos may be extended to generate complements of the different barcode sequences, and a ligase joins the adjacent ends to form a closed circular polynucleotide, as depicted in FIG. 3. The circular polynucleotide may be amplified and detected.

FIG. 3 illustrates a circular oligonucleotide including the complements of two unique molecular identifying sequences, UMI-l′ and UMI-2′.

FIGS. 4A-4D provide embodiments to generate amplification products. FIG. 4A illustrates annealing a primer (12) to the circular oligonucleotide and, with a strand displacing enzyme (13), extending to generate a concatemer of tandem repeats of the sequences of interest (14), where concatemer (14) is shown in FIG. 4B. FIG. 4B depicts the amplification products following rolling circle amplification, generating an amplification product including one or more copies of the first sequence, the first UMI sequence, the third sequence, the fourth sequence, the second UMI sequence, and the second sequence. The concatemer may be detected and sequenced in situ, for example according to the methods described herein (e.g., the G4X™ Platform). FIG. 4C illustrates an embodiment wherein the universal padlock probe (PLP) oligonucleotide includes a first primer binding sequence (PBS1) and a second primer binding sequence (PBS2′), thus according to the methods described herein the resulting circular oligonucleotide includes the primer binding sequences. A primer complementary to the first primer binding sequence may be used to generate a complementary single-stranded oligonucleotide including the first primer binding sequence (PBS1′), the first sequence, the first UMI sequence, the third sequence, the fourth sequence, the second UMI sequence, the second sequence, and the second primer binding sequence (PBS2), as illustrated in FIG. 4D. The single-stranded nucleotide may be further processed and sequenced on a next-generation sequencing device, such as the G4® Sequencing Platform.

FIG. 5 illustrates an embodiment wherein the association of two proteins (i.e., Protein A with Protein B), as well as the location of their association within the cell, can be detected in parallel with, and distinguished from, that of a different protein-protein association (i.e., Protein C with Protein B), via the generation of distinct barcode combinations. In this particular example, Protein B's association with Protein A can be distinguished from its association with Protein C due to the combination of the Protein B barcode, with that of either Protein A or Protein C, to generate unique Protein A-Protein B (15) and Protein C-Protein B (16) barcodes, respectively. The resulting localized, circular RCA templates can then be amplified, and then sequenced, in situ, thus revealing the identities and locations of the different interacting proteins.

FIGS. 6A-6C illustrate additional embodiments. FIGS. 6A-B show how the proximity of two proteins (i.e., Protein A with Protein C), that are associated with each other indirectly through other proteins within the same complex (i.e., Protein B and/or Protein D), can be detected via technology described herein. FIG. 6C shows how these methods can be used to determine the proximity of proteins with other types of compounds as well (i.e., carbohydrates). As such the posttranslational modifications of these proteins (i.e., glycosylation), and the correlation the modifications may have with protein-protein associations and localizations, may be determined.

### DETAILED DESCRIPTION

The aspects and embodiments described herein relate to compositions and method for detection of biomolecules, including glycosylation and interaction information.

### I. Definitions

All patents, patent applications, articles and publications mentioned herein, both supra and infra, are hereby expressly incorporated herein by reference in their entireties.

The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. Sec, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices, and materials similar or equivalent to those described herein can be used in the practice of embodiments of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure, or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The terms “attached,” “bind,” and “bound” as used herein are used in accordance with their plain and ordinary meanings and refer to an association between atoms or molecules. The association can be direct or indirect. For example, attached molecules may be directly bound to one another, e.g., by a covalent bond or non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

“Specific binding” as used herein, refers to a binding event which is selective between two molecules. A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. In embodiments, the K_{D} (equilibrium dissociation constant) between two specific binding molecules is less than 10⁻⁶ M, less than 10⁻⁷M, less than 10⁻⁸ M, less than 10⁻⁹M, less than 10⁻⁹M, less than 10⁻¹¹ M, or less than about 10⁻¹² M or less. As used herein, a “specific binding reagent” or “specific binding agent” refers to an agent that binds specifically to a particular biomolecule (e.g., carbohydrate, cell surface receptor, protein, nucleic acid, or lipid molecule). Examples of a specific binding reagent include, but are not limited to, an antibody or target-specific oligonucleotide.

As used herein, the term “contacting” is used in accordance with its plain ordinary meaning and refers to the process of allowing at least two distinct species (e.g., chemical compounds, biomolecules, nucleotides, binding reagents, or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, a protein (e.g., an antibody), or enzyme.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances, two or more associated species are “tethered”, “coated”, “attached”, or “immobilized” to one another or to a common solid or semisolid support (e.g., a receiving substrate). An association may refer to a relationship, or connection, between two entities. For example, a barcode sequence may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. In embodiments, a proximity probe is associated with a particular barcode, such that identifying the barcode identifies the probe with which it is associated. Because the proximity probe specifically binds to a target, identifying the barcode thus identifies the target.

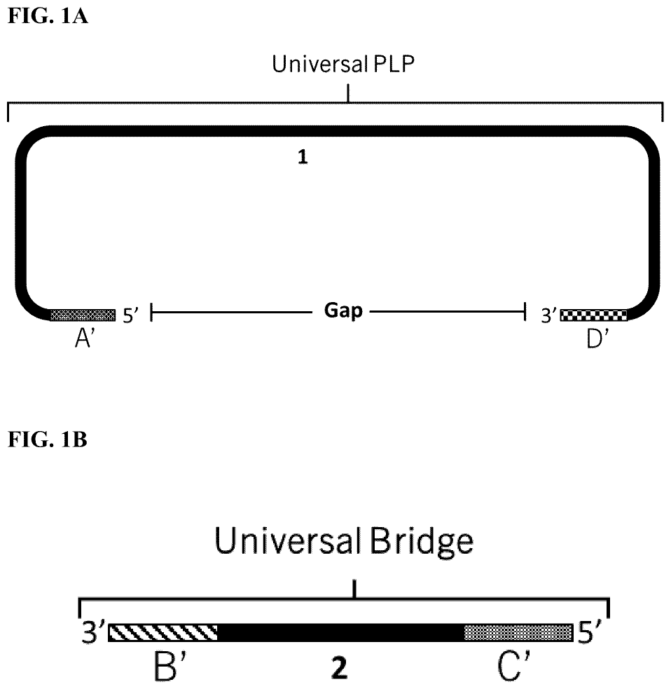

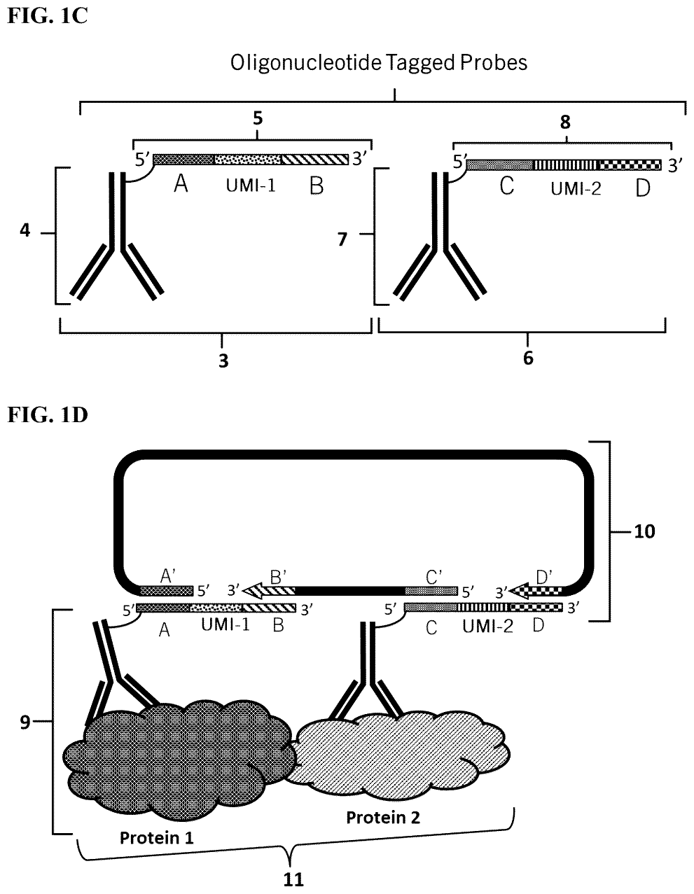

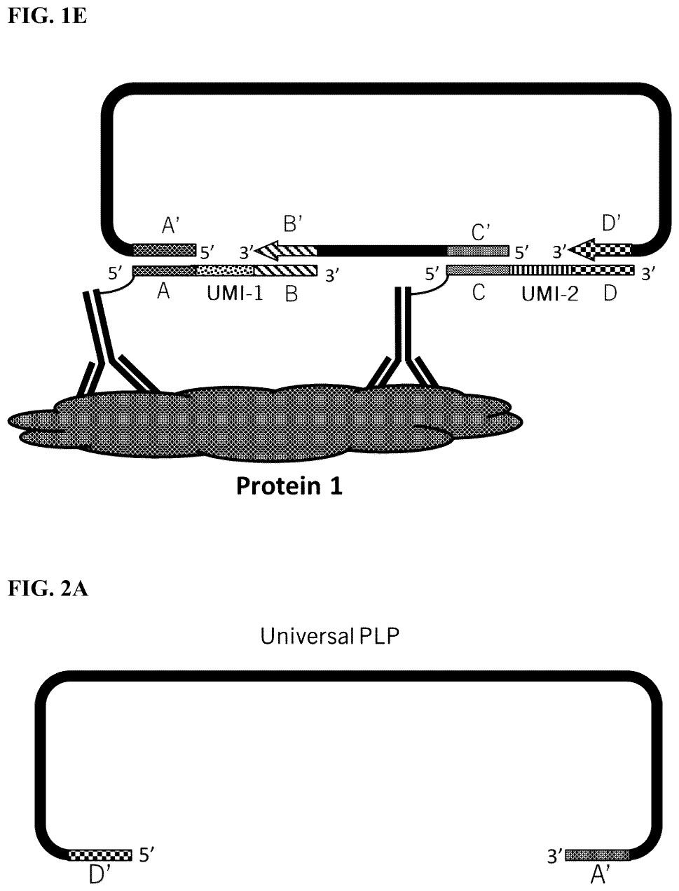

As used herein, the term “proximity probe” or “probe” (e.g., a first probe or a second probe described herein) is used in accordance with its plain ordinary meaning and refers to a specific binding agent (e.g., an antibody) attached to an oligonucleotide. In embodiments, pairs or sets of proximity probes can be employed to target multiple biomolecules of interest. Alternatively, in embodiments, a pair of proximity probes may be employed for a single biomolecule of interest. When different proximity probes harboring complementary oligonucleotides are adjacent, these oligonucleotides can be ligated, extended, and/or amplified to facilitate the detection of proteins and/or complexes. In embodiments, pairs or sets of probes can be employed to target multiple biomolecules of interest (e.g., as shown using a first probe or a second probe to detect different biomolecules proximal to each other in FIGS. 1D, 2D, 5, and 6A). Alternatively, in embodiments, a pair of probes may be employed for a single biomolecule of interest (e.g., as shown using a first probe or a second probe to different regions on the same biomolecule in FIGS. 1E, 2E, and 6C). When different proximity probes harboring complementary oligonucleotides are adjacent, these oligonucleotides can be ligated, extended, and/or amplified to facilitate the detection of proteins and/or complexes. Examples of biological assay that utilize proximity probes include proximity ligation assay (PLA) and proximity extension assay (PEA). In addition, proximity probes or probes include an antibody fragment, an affimer, an aptamer, or nucleic acid to facilitate interaction between biomolecule of interest.

As used herein, the term “affimer” is used in accordance with its plain ordinary meaning and refers to non-antibody binding proteins. These small proteins bind to target proteins with nanomolar affinity to facilitate the labelling of biomolecules in cells. An example of affimer includes, and is not limited to, Affimer® Technology, which is commercialized by Avacta® for diagnostic applications.

As used herein, the term “aptamer” is used in accordance with its plain ordinary meaning and refers to oligonucleotide or peptide molecules that bind to a specific target molecule. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions. In embodiments, peptide aptamers consist of one (or more) short variable peptide domains, attached at both ends to a protein scaffold. Aptamers may be designed with any combination of the base modified nucleotides desired. Aptamers to a given target include nucleic acids that are identified from a candidate mixture of nucleic acids, where the aptamer is a ligand of the target, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target with a much higher degree of affinity than it binds to other, non-target, components in a mixture or sample. An aptamer can be identified using any known method, including the SELEX process. Sec, e.g., U.S. Pat. No. 5,475,096 entitled “Nucleic Acid Ligands”. Once identified, an aptamer can be prepared or synthesized in accordance with any known method, including chemical synthetic methods and enzymatic synthetic methods.

Nucleic acid aptamers are nucleic acid species that are typically the product of engineering through repeated rounds of in vitro selection, such as SELEX (systematic evolution of ligands by exponential enrichment), to bind to various molecular targets such as small molecules, proteins, nucleic acids, and even cells, tissues, and organisms. At the molecular level, aptamers bind to its target site through non-covalent interactions. Aptamers bind to these specific targets because of electrostatic interactions, hydrophobic interactions, and their complementary shapes. In embodiments, peptide aptamers are artificial proteins selected or engineered to bind specific target molecules. These proteins may include or consist of one or more peptide loops of variable sequence displayed by a protein scaffold. They are typically isolated from combinatorial libraries and often subsequently improved by directed mutation or rounds of variable region mutagenesis and selection. An example of an aptamer is Macugen, which is a pegylated aptamer that targets the growth factor, VEFG165. (Sec Ni et al. ACS Appl Mater Interfaces. 2021 Mar. 3;13 (8): 9500-9519 and Song et al. Sensors (Basel). 2012; 12 (1): 612-31).

As used herein, the term “complement,” refers to a nucleotide (e.g., RNA or DNA) or a sequence of nucleotides capable of base pairing with a complementary nucleotide or sequence of nucleotides. For example, complementarity exists between the two strands of a double stranded DNA molecule or between an oligonucleotide primer and a primer binding site on a single stranded nucleic acid when a nucleotide (e.g., RNA or DNA) or a sequence of nucleotides is capable of base pairing with a respective cognate nucleotide or cognate sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence, only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Another example of complementary sequences are a template sequence and an amplicon sequence polymerized by a polymerase along the template sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often comprise nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, one or both sequences in a pair of complementary sequences form portions of longer polynucleotides, which may or may not include additional regions of complementarity.

“Hybridize” is used in accordance with its ordinary meaning and refers to the annealing or binding of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution. The primary interaction between polynucleotide strands typically is nucleotide base specific, e.g., A:T, A:U, and G:C, by Watson-Crick and Hoogsteen-type hydrogen bonding. Base-stacking and hydrophobic interactions also may contribute to duplex stability. Hybridization conditions may include salt concentrations of less than about 1 M, more usually less than about 500 mM, or less than about 200 mM. A hybridization buffer may include a buffered salt solution such as 5% SSPE or another suitable buffer known in the art. Hybridization temperatures may be as low as 5° C., but are typically greater than 22° C., and more typically greater than about 30° C., and typically in excess of 37° C. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

As used herein, the term “adjacent,” refers to two nucleotide sequences in a nucleic acid molecule, and can refer to nucleotide sequences separated by 0 to about 20 nucleotides, more specifically, in a range of about 1 to about 10 nucleotides, or to sequences that directly abut one another. As those of skill in the art appreciate, two nucleotide sequences that are ligated together will generally directly abut one another.

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives, or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward, or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the terms “polynucleotide template” and “template polynucleotide” refer to any polynucleotide molecule that may be bound by a polymerase and utilized as a template for nucleic acid synthesis. As used herein, the term “polynucleotide primer” refers to any polynucleotide molecule that may hybridize to a polynucleotide template, be bound by a polymerase, and be extended in a template-directed process for nucleic acid synthesis, such as in a PCR or sequencing reaction. Polynucleotide primers attached to a core polymer within a core are referred to as “core polynucleotide primers.” A primer can be of any length depending on the particular technique it will be used for. For example, amplification primers are generally between 10 and 40 nucleotides in length. The length and complexity of the nucleic acid fixed onto the nucleic acid template may vary. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide.

In general, the terms “target polynucleotide” and “target nucleic acid” are used interchangeably herein refer to a nucleic acid molecule or polynucleotide in a starting population of nucleic acid molecules having a target sequence whose presence, amount, and/or nucleotide sequence, or changes in one or more of these, are desired to be determined. In general, the terms “target sequence” and “target region”, refer to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In embodiments, the template polynucleotide includes a target nucleic acid sequence and one or more barcode sequences. In embodiments, the template polynucleotide is a barcode sequence.

The term “adapter” as used herein refers to any oligonucleotide that can be ligated to a nucleic acid molecule, thereby generating nucleic acid products that can be sequenced on a sequencing platform (e.g., an Illumina® or Singular Genomics® sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., primer sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification, or sequencing. In embodiments, the adapter can include an index sequence (also referred to as barcode or tag) to assist with downstream error correction, identification, or sequencing. In some embodiments, an adapter is hairpin adapter. In some embodiments, a hairpin adapter comprises a single nucleic acid strand comprising a stem-loop structure. In some embodiments, a hairpin adapter comprises a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter comprises a stem portion (i.e., stem) and a loop, wherein the stem portion is substantially double stranded thereby forming a duplex. In some embodiments, the loop of a hairpin adapter comprises a nucleic acid strand that is not complementary (e.g., not substantially complementary) to itself or to any other portion of the hairpin adapter. In some embodiments, a method herein comprises ligating a first adapter to a first end of a double stranded nucleic acid, and ligating a second adapter to a second end of a double stranded nucleic acid. In some embodiments, the first adapter and the second adapter are different. For example, in certain embodiments, the first adapter and the second adapter may comprise different nucleic acid sequences or different structures. In some embodiments, the first adapter is a Y-adapter and the second adapter is a hairpin adapter. In some embodiments, the first adapter is a hairpin adapter and a second adapter is a hairpin adapter. In certain embodiments, the first adapter and the second adapter may comprise different primer binding sites, different structures, and/or different capture sequences (e.g., a sequence complementary to a capture nucleic acid). In some embodiments, some, all or substantially all of the nucleic acid sequence of a first adapter and a second adapter are the same. In some embodiments, some, all or substantially all of the nucleic acid sequence of a first adapter and a second adapter are substantially different.

As used herein, the terms “bridge oligonucleotide,” “bridge oligo”, and “bridging oligonucleotide” refer to an oligonucleotide with a first region of complementarity to a first independent polynucleotide at the 5′ end of the bridge oligonucleotide, and a second region of complementarity to a second independent polynucleotide at the 3′ end of the bridge oligonucleotide. In embodiments, the first independent polynucleotide and the second independent polynucleotide are different or substantially different. In embodiments, the first independent polynucleotide and the second independent polynucleotide are the same or substantially the same.

A “targeted primer sequence” refers to a nucleic acid sequence that is complementary to a known nucleic acid region (e.g., complementary to a universally conserved region, or complementary sequences to target specific genes or mutations that have relevancy to a particular cancer phenotype). The hybridization sequences may include sequences designed through computational software, e.g., Primer BLAST, LaserGene (DNAStar), Oligo (National Biosciences, Inc.), MacVector (Kodak/IBI) or the GCG suite of programs to optimize desired properties. In embodiments, the hybridization sequence includes a limited-diversity sequence. A “limited-diversity sequence” refers to a nucleic acid sequence that includes random nucleotide regions and fixed nucleotide regions (e.g., NNANN, ANNTN, TNCNA, etc., wherein N represents random nucleotides and A, T, C, G represent fixed nucleotides). In embodiments, each hybridization sequence is composed of 3 random nucleotides and 1 to 2 non-random nucleotides. In embodiments, each hybridization sequence is composed of 4 random nucleotides and 1 to 2 non-random nucleotides.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate,- or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Other analog nucleic acids include bis-locked nucleic acids (bisLNAs; e.g., including those described in Moreno PMD et al. Nucleic Acids Res. 2013; 41 (5): 3257-73), twisted intercalating nucleic acids (TINAs; e.g., including those described in Doluca O et al. Chembiochem. 2011; 12 (15): 2365-74), bridged nucleic acids (BNAs; e.g., including those described in Soler-Bistue A et al. Molecules. 2019; 24 (12): 2297), 2′-O-methyl RNA: DNA chimeric nucleic acids (e.g., including those described in Wang S and Kool ET. Nucleic Acids Res. 1995; 23 (7): 1157-1164), minor groove binder (MGB) nucleic acids (e.g., including those described in Kutyavin IV et al. Nucleic Acids Res. 2000; 28 (2): 655-61), morpholino nucleic acids (e.g., including those described in Summerton J and Weller D. Antisense Nucleic Acid Drug Dev. 1997; 7 (3): 187-95), C5-modified pyrimidine nucleic acids (e.g., including those described in Kumar P et al. J. Org. Chem. 2014; 79 (11): 5047-5061), peptide nucleic acids (PNAs; e.g., including those described in Gupta A et al. J. Biotechnol. 2017; 259:148-59), and/or phosphorothioate nucleotides (e.g., including those described in Eckstein F. Nucleic Acid Ther. 2014; 24 (6): 374-87).

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytidine, thymidine or uracil and the N-4 position on cytosine.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl) phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules (e.g., an internal cleavable site). In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al., Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al., J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase. Cleavage agents used in methods described herein may be selected from nicking endonucleases, DNA glycosylases, or any single-stranded cleavage agents described in further detail elsewhere herein. Enzymes for cleavage of single-stranded DNA may be used for cleaving heteroduplexes in the vicinity of mismatched bases, D-loops, heteroduplexes formed between two strands of DNA which differ by a single base, an insertion or deletion. Mismatch recognition proteins that cleave one strand of the mismatched DNA in the vicinity of the mismatch site may be used as cleavage agents. Nonenzymatic cleaving may also be done through photodegredation of a linker introduced through a custom oligonucleotide used in a PCR reaction.

As used herein, the term “cleavable complement” refers to a nucleotide (e.g., RNA or DNA) or a sequence of nucleotides capable of base pairing with a complementary nucleotide or sequence of nucleotides, wherein the complementary nucleotide or sequence of nucleotides includes a cleavable site, and the cleavable complement also includes a complement to the cleavable site. In embodiments, the cleavable complement of the cleavable site and the cleavable site are cleaved by the same mechanism (e.g., restriction enzyme digestion of the duplexed cleavable site and cleavable complement of the cleavable site).

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

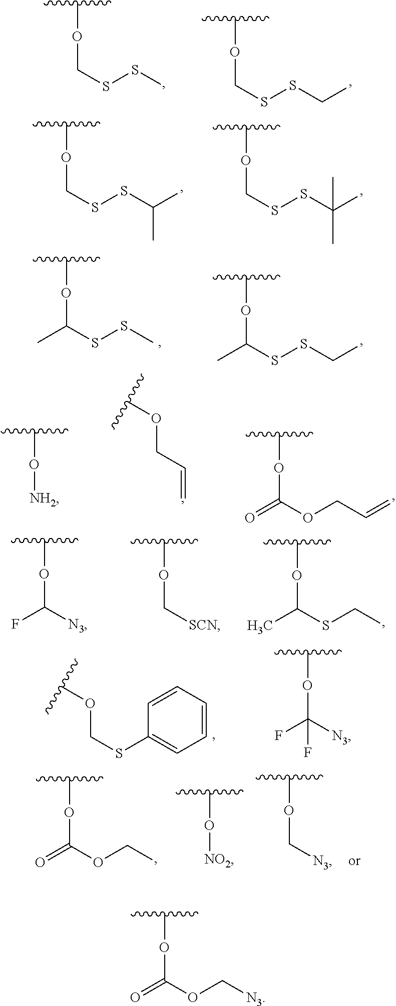

A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes. Non-limiting examples of detectable labels include labels including fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF® dyes (Biotium, Inc.), Alexa Fluor® dyes (Thermo Fisher), DyLight® dyes (Thermo Fisher), Cy® dyes (GE Healthscience), IRDye® dyes (Li-Cor Biosciences, Inc.), and HiLyte™ dyes (Anaspec, Inc.). In embodiments, the label is a fluorophore.

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF® dyes (Biotium, Inc.), Alexa Fluor® dyes (Thermo Fisher), DyLight® dyes (Thermo Fisher), Cy® dyes (GE Healthscience), IRDyes® dyes (Li-Cor Biosciences, Inc.), and HiLyte™ dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa Fluor® dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy®3). In embodiments, the cyanine moiety has 5 methine structures (i.e., cyanine 5 or Cy®5). In embodiments, the cyanine moiety has 7 methine structures (i.e., cyanine 7 or Cy®7).

The terms “detect” and “detecting” as used herein refer to the act of viewing (e.g., imaging, indicating the presence of, quantifying, or measuring (e.g., spectroscopic measurement), an agent based on an identifiable characteristic of the agent, for example, the light emitted from the present compounds. For example, the compound described herein can be bound to an agent, and, upon being exposed to an absorption light, will emit an emission light. The presence of an emission light can indicate the presence of the agent. Likewise, the quantification of the emitted light intensity can be used to measure the concentration of the agent. In embodiments, detecting includes detecting a light emission in the near-infrared spectrum. In embodiments, detecting includes detecting a light emission with a wavelength from 600 nm-900 nm. In embodiments, detecting includes detecting a light emission with a wavelength from 600 nm-1450 nm. In embodiments, detecting includes detecting a light emission with a wavelength from 1000 nm-1700 nm. In embodiments, detecting includes detecting a light emission in the “imaging window,” which refers to a range of wavelengths where tissue autofluorescence is minimal and the absorption and emission of light in tissue results in minimal light scattering (see, e.g., Pansare et al. Chem Mater. 2012 Mar. 13; 24 (5): 812-827 and Wang et al. ACS Cent Sci. 2020 Aug. 26; 6 (8): 1302-1316).

The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine, and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group-OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula:

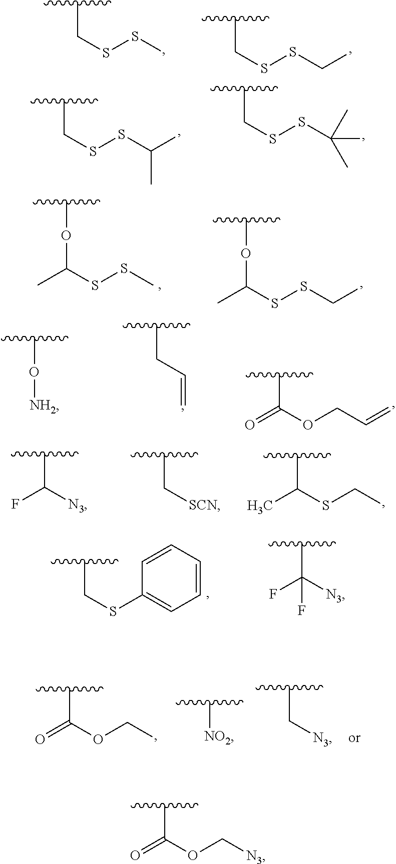

wherein the 3′ oxygen of the nucleotide is not shown in the formulae above. The term “allyl” as described herein refers to an unsubstituted methylene attached to a vinyl group (i.e., —CH═CH₂). In embodiments, the reversible terminator moiety is attached to the 3′-oxygen of the nucleotide, having the formula:

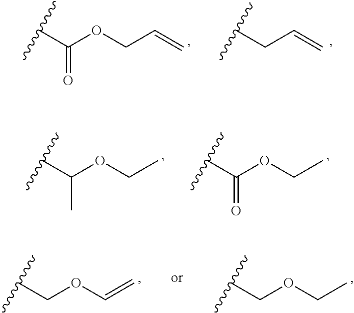

wherein the 3′ oxygen of the nucleotide is not shown in the formulae above. In embodiments, the reversible terminator moiety is

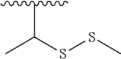 [SMILES: *C(C)SSC]

as described in U.S. Pat. No. 10,738,072, which is incorporated herein by reference for all purposes. For example, a nucleotide including a reversible terminator moiety may be represented by the formula:

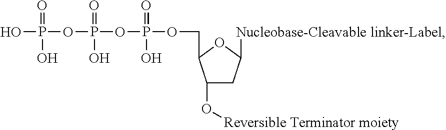

where the nucleobase is adenine or adenine analogue, thymine or thymine analogue, guanine or guanine analogue, or cytosine or cytosine analogue.

In some embodiments, a nucleic acid (e.g., a probe or a primer) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of nucleic acid molecules is unique, such that sequencing reads including the barcode can be identified as originating from a single sample polynucleotide molecule on the basis of the barcode alone. In other embodiments, individual barcode sequences may be used more than once, but oligonucleotides including the duplicate barcodes are associated with different sequences and/or in different combinations of barcoded oligonucleotides, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 1, 2, 3, 4, 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined. In embodiments, the barcode sequence is a nucleic acid sequence (e.g., 8 to 24 nucleotides) from a known set of barcode sequences. In embodiments, each barcode sequence is unique within the known set of barcodes. In embodiments, the barcodes are selected to form a known set of barcodes, e.g., the set of barcodes may be distinguished by a particular Hamming distance. In embodiments, a barcode is associated with a particular proximity probe. In embodiments, a set of barcodes is associated with a particular probe or target (e.g., a set of barcodes may be associated with a protein). In embodiments, a set of different UMIs may be coupled to a plurality of different targets, and each of those targets may be identified using the particular UMI. One example of a UMI is a “barcode”. In embodiments, a barcode is a nucleotide, nucleotide sequence, or identifier, that conveys or is capable of conveying information (e.g., information about an analyte in a cell or tissue.

In embodiments, a nucleic acid (e.g., a probe or primer) includes a sample barcode. In general, a “sample barcode” is a nucleotide sequence that is sufficiently different from other sample barcode to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

As used herein, the terms “biomolecule”, “analyte”, or “target molecule” refers to an agent (e.g., a compound, macromolecule, or small molecule), and the like derived from a biological system (e.g., an organism, a cell, or a tissue) to be detected using the methods and compositions described herein. The biomolecule (i.e., target molecule) may contain multiple individual components that collectively construct the biomolecule, for example, in embodiments, the biomolecule (i.e., target molecule) is a polynucleotide wherein the polynucleotide is composed of nucleotide monomers. The biomolecule (i.e., target molecule) may be or may include DNA, RNA, organelles, carbohydrates, lipids, proteins, or any combination thereof. These components may be extracellular. In some examples, the biomolecule (i.e., target molecule) may be referred to as a clump or aggregate of combinations of components. In some instances, the biomolecule (i.e., target molecule) may include one or more constituents of a cell but may not include other constituents of the cell. In embodiments, a biomolecule (i.e., target molecule) is a molecule produced by a biological system (e.g., an organism). The biomolecule (i.e., target molecule) may be any substance (e.g., molecule) or entity that is desired to be detected by the method of the invention. The biomolecule is the “target” of the assay method of the invention. The biomolecule (i.e., target molecule) may accordingly be any compound that may be desired to be detected, for example a peptide or protein, or nucleic acid molecule or a small molecule, including organic and inorganic molecules. The biomolecule (i.e., target molecule) may be a cell or a microorganism, including a virus, or a fragment or product thereof. Biomolecules of particular interest (i.e., target molecules) may thus include proteinaceous molecules such as peptides, polypeptides, proteins or prions or any molecule which includes a protein or polypeptide component, etc., or fragments thereof. The biomolecule (i.e., target molecule) may be a single molecule or a complex that contains two or more molecular subunits, which may or may not be covalently bound to one another, and which may be the same or different. Thus, in addition to cells or microorganisms, such a complex biomolecule (i.e., target molecule) may also be a protein complex. Such a complex may thus be a homo- or hetero-multimer. Aggregates of molecules e.g., proteins may also be target analytes, for example aggregates of the same protein or different proteins. The biomolecule (i.e., target molecule) may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules.

In embodiments, the target molecules are proteins or carbohydrates. In embodiments, the target molecules are proteins. In embodiments, the target molecules are carbohydrates. In embodiments when the target molecules are proteins and/or carbohydrates, the method includes contacting the proteins with a specific binding reagent, wherein the specific binding reagent comprises an oligonucleotide barcode. In embodiments, the specific binding reagent comprises an antibody, single-chain Fv fragment (scFv), antibody fragment-antigen binding (Fab), or an aptamer. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminocthyl (DEAE)-dextran, cholesterol, or a sterol moiety. In embodiments, the specific binding reagent interacts (e.g., contacts, or binds) with one or more specific binding reagents on the cell surface. Carbohydrate-specific antibodies are known in the art, see for example Kappler, K., Hennet, T. Genes Immun 21, 224-239 (2020).

As used herein, “biomaterial” refers to any biological material produced by an organism. In some embodiments, biomaterial includes secretions, extracellular matrix, proteins, lipids, organelles, membranes, cells, portions thereof, and combinations thereof. In some embodiments, cellular material includes secretions, extracellular matrix, proteins, lipids, organelles, membranes, cells, portions thereof, and combinations thereof. In some embodiments, biomaterial includes viruses. In some embodiments, the biomaterial is a replicating virus and thus includes virus infected cells. In embodiments, a biological sample includes biomaterials.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9°N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9°N polymerase (exo-) A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol A DNA polymerase, Pol σ DNA polymerase, Pol α DNA polymerase, Pol δ DNA polymerase, Pol & DNA polymerase, Pol η DNA polymerase, Pol l DNA polymerase, Pol κ DNA polymerase, Pol (DNA polymerase, Pol γ DNA polymerase, Pol θ DNA polymerase, Pol ν DNA polymerase, or a thermophilic nucleic acid polymerase (e.g. Therminator γ, 9°N polymerase (exo-), Therminator™ II, Therminator™ III, or Therminator™ IX). In embodiments, the DNA polymerase is a modified archacal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884.

As used herein, the term “exonuclease activity” is used in accordance with its ordinary meaning in the art, and refers to the removal of a nucleotide from a nucleic acid by an enzyme (e.g., a DNA polymerase with exonuclease activity). For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at either the 3′ end of a polynucleotide chain to excise the nucleotide. In embodiments, 3′-5′ exonuclease activity refers to the successive removal of nucleotides in single-stranded DNA in a 3′->5′ direction, releasing deoxyribonucleoside 5′-monophosphates one after another. Methods for quantifying exonuclease activity are known in the art, see for example Southworth et al., PNAS Vol 93, 8281-8285 (1996).

As used herein, the term “endonuclease” refers to an enzyme capable of cleaving the phosphodiester bond within a polynucleotide chain. The polynucleotide may be double-stranded DNA (dsDNA), single-stranded DNA (ssDNA), RNA, double-stranded hybrids of DNA and RNA, and synthetic DNA (for example, containing bases other than A, C, G, and T). An endonuclease may cut a polynucleotide symmetrically, leaving “blunt” ends, or in positions that are not directly opposing, creating overhangs, which may be referred to as “sticky ends.” An endonuclease may cut a double-stranded polynucleotide on a single strand. The methods and compositions described herein may be applied to cleavage sites generated by endonucleases. In some alternatives of the system, the system can further provide nucleic acids that encode an endonuclease, such as Cas9, TALEN, or MegaTAL, or a fusion protein comprising a domain of an endonuclease, for example, Cas9, TALEN, or MegaTAL, or one or more portion thereof. These examples are not meant to be limiting and other endonucleases and alternatives of the system and methods comprising other endonucleases and variants and modifications of these exemplary alternatives are possible without undue experimentation. All such variations and modifications are within the scope of the current teachings.

As used herein, the term “nicking endonuclease” refers to any enzyme, naturally occurring or engineered, that is capable of breaking a phosphodiester bond on a single DNA strand, leaving a 3′-hydroxyl. Nicking endonucleases can be engineered by modifying restriction enzymes to eliminate cutting activity for one DNA strand, or produced by fusing a nicking subunit to a DNA binding domain, for example, zinc fingers and DNA recognition domains from transcription activator-like effectors.

As used herein, “nick” generally refers to enzymatic cleavage of only one strand of a double-stranded nucleic acid at a particular region, while leaving the other strand intact, regardless of whether one or more bases are removed. In some cases, one or more bases are removed while in other cases no bases are removed and only phosphodiester bonds are broken. In some instances, such cleavage events leave behind intact double-stranded regions lacking nicks that are a short distance apart from each other on the double-stranded nucleic acid, for example a distance of about or at least about 1, 2, 3, 4, 5, 6, 7, 8, 9, 10, 11, 12, 13, 14, 15 bases or more. In some cases, the distance between the intact double-stranded regions is equal to or less than 15, 14, 13, 12, 11, 10, 9, 8, 7, 6, 5, 4, 3, or 2 bases. In some instances, the distance between the intact double-stranded regions is 2 to 10 bases, 3 to 9 bases, or 4 to 8 bases.

As used herein, the term “incorporating” or “chemically incorporating,” when used in reference to a primer and cognate nucleotide, refers to the process of joining the cognate nucleotide to the primer or extension product thereof by formation of a phosphodiester bond.

In embodiments, a target polynucleotide is a cell-free polynucleotide. In general, the terms “cell-free,” “circulating,” and “extracellular” as applied to polynucleotides (e.g., “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g., apoptosis or necrosis) or cell shedding, releasing polynucleotides into surrounding body fluids or into circulation. Accordingly, cell-free polynucleotides may be isolated from a non-cellular fraction of blood (e.g., serum or plasma), from other bodily fluids (e.g., urine), or from non-cellular fractions of other types of samples.

A nucleic acid can be amplified by a suitable method. The term “amplified” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction includes a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that includes a polymerase chain reaction (PCR). Conditions conducive to amplification (i.e., amplification conditions) are well known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

Amplification according to the present teachings encompasses any means by which at least a part of at least one target nucleic acid is reproduced, typically in a template-dependent manner, including without limitation, a broad range of techniques for amplifying nucleic acid sequences, cither linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4 (1): 41-7, U.S. Pat. Nos. 6,027,998; 6,605,451, Barany et al., PCT Publication No. WO 97/31256; Wenz et al., PCT Publication No. WO 01/92579; Day et al., Genomics, 29 (1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Barany, Proc. Natl. Acad. Sci. USA 88:188-93 (1991); Bi and Sambrook, Nucl. Acids Res. 25:2924-2951 (1997); Zirvi et al., Nucl. Acid Res. 27: c40i-viii (1999); Dean et al., Proc Natl Acad Sci USA 99:5261-66 (2002); Barany and Gelfand, Genc 109:1-11 (1991); Walker et al., Nucl. Acid Res. 20:1691-96 (1992); Polstra et al., BMC Inf. Dis. 2:18-(2002); Lage et al., Genome Res. 2003 February; 13 (2): 294-307, and Landegren et al., Science 241:1077-80 (1988), Demidov, V., Expert Rev Mol Diagn. 2002 November; 2 (6): 542-8., Cook et al., J Microbiol Methods. 2003 May; 53 (2): 165-74, Schweitzer et al., Curr Opin Biotechnol. 2001 February; 12 (1): 21-7, U.S. Pat. Nos. 5,830,711, 6,027,889, 5,686,243, PCT Publication No. WO0056927A3, and PCT Publication No. WO9803673A1.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge PCR amplification, emulsion PCR, WildFire amplification (e.g., US patent publication US20130012399), the like or combinations thereof.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example, an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Provided herein are methods and compositions for analyzing a sample (e.g., sequencing nucleic acids within a sample). A sample (e.g., a sample including nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, car, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, car, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). The biological targets or molecules to be detected can be any biological molecules including but not limited to proteins, nucleic acids, lipids, carbohydrates, ions, or multicomponent complexes containing any of the above. Examples of subcellular targets include organelles, e.g., mitochondria, Golgi apparatus, endoplasmic reticulum, chloroplasts, endocytic vesicles, exocytic vesicles, vacuoles, lysosomes, etc. Exemplary nucleic acid targets can include genomic DNA of various conformations (e.g., A-DNA, B-DNA, Z-DNA), mitochondria DNA (mtDNA), mRNA, tRNA, rRNA, hRNA, miRNA, and piRNA.

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof.

A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the terms “sequencing”, “sequence determination”, and “determining a nucleotide sequence”, are used in accordance with their ordinary meaning in the art, and refer to determination of partial as well as full sequence information of the nucleic acid being sequenced, and particular physical processes for generating such sequence information. That is, the term includes sequence comparisons, fingerprinting, and like levels of information about a target nucleic acid, as well as the express identification and ordering of nucleotides in a target nucleic acid. The term also includes the determination of the identification, ordering, and locations of one, two, or three of the four types of nucleotides within a target nucleic acid. Sequencing produces one or more sequencing reads.

As used herein, the term “sequencing reaction mixture” is used in accordance with its plain and ordinary meaning and refers to an aqueous mixture that contains the reagents necessary to add a dNTP or dNTP analogue (e.g., a modified nucleotide) a DNA strand by a DNA polymerase. In embodiments, the sequencing reaction mixture includes a buffer. In embodiments, the buffer includes an acetate buffer, 3-(N-morpholino) propanesulfonic acid (MOPS) buffer, N-(2-Acctamido)-2-aminocthanesulfonic acid (ACES) buffer, phosphate-buffered saline (PBS) buffer, 4-(2-hydroxyethyl)-1-piperazincethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

As used herein, the term “sequencing cycle” is used in accordance with its plain and ordinary meaning and refers to binding and/or incorporating one or more nucleotides (e.g., a compound described herein) to the 3′ end of a polynucleotide with a polymerase, and detecting one or more labels that identify the one or more nucleotides. The sequencing may be accomplished by, for example, sequencing by synthesis, sequencing by binding, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes and other substances can be removed between steps by washing. Cycles may include repeating these steps, and the sequence of each cluster is read over the multiple repetitions.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 1, 5, 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic, or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic. In some embodiments, the alternating layers of polymeric gels described include a hydrophilic material.

As used herein, the term “hydrogel” refers to a three-dimensional polymeric structure that is substantially insoluble in water, but which is capable of absorbing and retaining water (e.g., large quantities of water) to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers. Hydrogels can contain over 99% water and may include natural or synthetic polymers, or a combination thereof. Hydrogels also possess a degree of flexibility very similar to natural tissue, due to their significant water content. A detailed description of suitable hydrogels may be found in published U.S. patent application 2010/0055733, herein incorporated by reference. By “hydrogel subunits” or “hydrogel precursors” is meant hydrophilic monomers, prepolymers, or polymers that can be crosslinked, or “polymerized”, to form a three-dimensional (3D) hydrogel network. In some embodiments, the alternating layers of polymeric gels described herein are hydrogels. Hydrogels may be prepared by cross-linking hydrophilic biopolymers or synthetic polymers. Thus, in some embodiments, the hydrogel may include a crosslinker. As used herein, the term “crosslinker” refers to a molecule that can form a three-dimensional network when reacted with the appropriate base monomers. Examples of the hydrogel polymers, which may include one or more crosslinkers, include but are not limited to, hyaluronans, chitosans, agar, heparin, sulfate, cellulose, alginates (including alginate sulfate), collagen, dextrans (including dextran sulfate), pectin, carrageenan, polylysine, gelatins (including gelatin type A), agarose, (meth) acrylate-oligolactide-PEO-oligolactide-(meth) acrylate, PEO—PPO-PEO copolymers (Pluronics), poly(phosphazene), poly(methacrylates), poly(N-vinylpyrrolidone), PL(G) A-PEO-PL(G) A copolymers, poly(ethylene iminc), polyethylene glycol (PEG)-thiol, PEG-acrylate, acrylamide, N,N′-bis(acryloyl) cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. Thus, for example, a combination may include a polymer and a crosslinker, for example polyethylene glycol (PEG)-thiol/PEG-acrylate, acrylamide/N,N′-bis(acryloyl) cystamine (BACy), or PEG/polypropylene oxide (PPO). In embodiments, the hydrogel includes chemical crosslinks (e.g., intermolecular or intramolecular joining of two or more molecules by a covalent bond) and may be referred to as a chemical hydrogel. In embodiments, the hydrogel includes physical crosslinks (e.g., intermolecular or intramolecular joining of two or more molecules by a non-covalent bond) and may be referred to as a physical hydrogel. In embodiments, the physical hydrogel include one or more crosslinks including hydrogen bonds, hydrophobic interactions, and/or polymer chain entanglements.

As used herein, the term “substrate” refers to a solid support material. The substrate can be non-porous or porous. The substrate can be rigid or flexible. As used herein, the terms “solid support” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently, or non-covalently). A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of the particle to maximize the contact between as substantially circular particle. In embodiments, the wells of an array are randomly located such that nearest neighbor features have random spacing between each other. Alternatively, in embodiments the spacing between the wells can be ordered, for example, forming a regular pattern. The term solid substrate is encompassing of a substrate (e.g., a flow cell) having a surface including a polymer coating covalently attached thereto. In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

The term “microplate”, or “multiwell container” as used herein, refers to a substrate including a surface, the surface including a plurality of reaction chambers separated from each other by interstitial regions on the surface. In embodiments, the microplate has dimensions as provided and described by American National Standards Institute (ANSI) and Society for Laboratory Automation and Screening (SLAS); for example, the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins, or polynucleotides in a cell.

The reaction chambers may be provided as wells of a multiwell container (alternatively referred to as reaction chambers), for example a microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches, and includes a plurality of 6 mm diameter wells. In embodiments, the microplate is about 5 inches by about 3.33 inches, and includes a plurality of 7 mm diameter wells. In embodiments, the microplate is about 5 inches by about 3.33 inches, and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches, and includes a plurality of 8 mm diameter wells. In embodiments, the microplate is a flat glass or plastic tray in which an array of wells are formed, wherein each well can hold between from a few microliters to hundreds of microliters of fluid reagents and samples. In embodiments, the microplate has a rectangular shape that measures 127.7 mm+0.5 mm in length by 85.4 mm+0.5 mm in width, and includes 6, 12, 24, 48, or 96 wells, wherein each well has an average diameter of about 5-7 mm. In embodiments, the microplate has a rectangular shape that measures 127.7 mm+0.5 mm in length by 85.4 mm+0.5 mm in width, and includes 6, 12, 24, 48, or 96 wells, wherein each well has an average diameter of about 6 mm.

The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, or star shaped (i.e., star shaped with any number of vertices). The cross section of a well taken orthogonally with the surface may be curved, square, polygonal, hyperbolic, conical, or angular. The wells of a microplate are available in different shapes, for example F-Bottom: flat bottom; C-Bottom: bottom with minimal rounded edges; V-Bottom: V-shaped bottom; or U-Bottom: U-shaped bottom. In embodiments, the well is substantially square. In embodiments, the well is square. In embodiments, the well is F-bottom. In embodiments, the microplate includes 24 substantially round flat bottom wells. In embodiments, the microplate includes 48 substantially round flat bottom wells. In embodiments, the microplate includes 96 substantially round flat bottom wells. In embodiments, the microplate includes 384 substantially square flat bottom wells.

The discrete regions (i.e., features, wells) of the microplate may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. In embodiments, interstitial regions have a surface material that differs from the surface material of the wells (e.g., the interstitial region contains a photoresist and the surface of the well is glass). In embodiments, interstitial regions have a surface material that is the same as the surface material of the wells (e.g., both the surface of the interstitial region and the surface of well contain a polymer or copolymer).

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

As used herein the term “determine” can be used to refer to the act of ascertaining, establishing, or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate reactive group 1 | Bioconjugate reactive group 2 | |
|---|---|---|
| (e.g., electrophilic bioconjugate | (e.g., nucleophilic bioconjugate | Resulting Bioconjugate |
| reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH2, —COOH, —N-hydroxysuccinimide, or -malcimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., malcimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., malcimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or malcimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with malcimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (1) metal silicon oxide bonding; (m) metal bonding to reactive phosphorus groups (e.g., phosphines) to form, for example, phosphate diester bonds.; (n) azides coupled to alkynes using copper catalyzed cycloaddition click chemistry; (o) biotin conjugate can react with avidin or streptavidin to form a avidin-biotin complex or streptavidin-biotin complex.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

As used herein, the term “control” or “control experiment” is used in accordance with its plain and ordinary meaning and refers to an experiment in which the subjects, cells, tissues, or reagents of the experiment are treated as in a parallel experiment except for omission of a procedure, reagent, or variable of the experiment. In some instances, the control is used as a standard of comparison in evaluating experimental effects. In embodiments, a control cell is the same cell type as the cell being examined, wherein the control cell does not include the variable or is subjected to conditions being examined.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein, the terms “incubate,” and “incubation” refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

As used herein, “biological activity” may include the in vivo activities of a compound or physiological responses that result upon in vivo administration of a compound, composition, or other mixture. Biological activity, thus, may encompass therapeutic effects and pharmaceutical activity of such compounds, compositions, and mixtures. Biological activities may be observed in vitro systems designed to test or use such activities.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

As used herein, a “plurality” refers to two or more.

As used herein the terms “automated” and “semi-automated” mean that the operations are performed by system programming or configuration with little or no human interaction once the operations are initiated, or once processes including the operations are initiated.

Provided herein are methods, systems, devices, and compositions for analyzing a sample in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, car, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, car, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation.

As used herein, the term “disease state” is used in accordance with its plain and ordinary meaning and refers to any abnormal biological state or aberration of a cell. The presence of a disease state may be identified by the same collection of biological constituents used to determine the cell's biological state. In general, a disease state will be detrimental to a biological system. A disease state may be a consequence of, inter alia, an environmental pathogen, for example a viral infection (e.g., HIV/AIDS, hepatitis B, hepatitis C, influenza, measles, etc.), a bacterial infection, a parasitic infection, a fungal infection, or infection by some other organism. A disease state may also be the consequence of some other environmental agent, such as a chemical toxin or a chemical carcinogen. As used herein, a disease state further includes genetic disorders wherein one or more copies of a gene is altered or disrupted, thereby affecting its biological function. Exemplary genetic diseases include, but are not limited to polycystic kidney disease, familial multiple endocrine neoplasia type I, neurofibromatosis, Tay-Sachs disease, Huntington's disease, sickle cell anemia, thalassemia, and Down's syndrome, as well as others (see, e.g., The Metabolic and Molecular Bases of Inherited Diseases, 7th ed., McGraw-Hill Inc., New York). Other exemplary diseases include, but are not limited to, cancer, hypertension, Alzheimer's disease, neurodegenerative diseases, and neuropsychiatric disorders such as bipolar affective disorders or paranoid schizophrenic disorders. Disease states are monitored to determine the level (e.g., the stage or progression) of one or more disease states of a subject and, more specifically, detect changes in the biological state of a subject which are correlated to one or more disease states (see, e.g., U.S. Pat. No. 6,218,122, which is incorporated by reference herein in its entirety). The methods provided herein may also be applicable to monitoring the disease state or states of a subject undergoing one or more therapies. Thus, provided herein, for example, are methods for determining or monitoring efficacy of a therapy or therapies (i.e., determining a level of therapeutic effect) upon a subject. In embodiments, the methods provided herein can be used to assess therapeutic efficacy in a clinical trial, e.g., as an early surrogate marker for success or failure in such a clinical trial. Within eukaryotic cells, there are hundreds to thousands of signaling pathways that are interconnected. For this reason, perturbations in the function of proteins within a cell have numerous effects on other proteins and the transcription of other genes that are connected by primary, secondary, and sometimes tertiary pathways. This extensive interconnection between the function of various proteins means that the alteration of any one protein is likely to result in compensatory changes in a wide number of other proteins. In particular, the partial disruption of even a single protein within a cell, such as by exposure to a drug or by a disease state which modulates the gene copy number (e.g., a genetic mutation), results in characteristic compensatory changes in the transcription of enough other genes that these changes in transcripts can be used to define a “signature” of particular transcript alterations which are related to the disruption of function, i.e., a particular disease state or therapy, even at a stage where changes in protein activity are undetectable.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A protein may refer to a protein expressed in a cell. A polypeptide, or a cell is “recombinant” when it is artificial or engineered, or derived from or contains an artificial or engineered protein or nucleic acid (e.g., non-natural, or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

An “antibody” (Ab) is a protein that binds specifically to a particular substance, known as an “antigen” (Ag). An “antibody” or “antigen-binding fragment” is an immunoglobulin that binds a specific “epitopc.” The term encompasses polyclonal, monoclonal, and chimeric antibodies. In nature, antibodies are generally produced by lymphocytes in response to immune challenge, such as by infection or immunization. An “antigen” (Ag) is any substance that reacts specifically with antibodies or T lymphocytes (T cells). An antibody may include the entire antibody as well as any antibody fragments capable of binding the antigen or antigenic fragment of interest. Examples include complete antibody molecules, antibody fragments, such as Fab, F(ab′)2, CDRs, VL, VH, and any other portion of an antibody which is capable of specifically binding to an antigen. Antibodies used herein are immunospecific for, and therefore specifically and selectively bind to, for example, proteins either detected (i.e., biological targets of interest) or used for detection (i.e., probes containing oligonucleotide barcodes) in the methods and devices as described herein. The term “monoclonal antibody” as used herein refers to an antibody obtained from a population of substantially homogeneous antibodies. That is, the individual antibodies including the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The “monoclonal antibodies” may also be isolated from phage antibody libraries using the techniques known in the art. Monoclonal antibodies, as the term is used herein, may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class.

The terms “cellular component” is used in accordance with its ordinary meaning in the art and refers to any organelle, nucleic acid, protein, or analyte that is found in a prokaryotic, eukaryotic, archacal, or other organismic cell type. Examples of cellular components (e.g., a component of a cell) include RNA transcripts, proteins, membranes, lipids, and other analytes.

A “gene” refers to a polynucleotide that is capable of conferring biological function after being transcribed and/or translated.

As used herein, the term “post translational modification” (PTM) refers to a modification of a protein following biosynthesis of that protein. Nonlimiting examples of PTMs include phosphorylation, methylation, nitrosylation, acetylation, and glycosylation.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic. As used herein, the term “multiplex” is used to refer to an assay in which multiple (i.e. at least two) different biomolecules are assayed at the same time, and more particularly in the same aliquot of the sample, or in the same reaction mixture. In embodiments, more than two different biomolecules are assayed at the same time. In embodiments, at least 2, 4, 6, 8, 10, 20, 50, 100, 200, 300, 400, 500, 600, 700, 800, 900, 1000, 1100, 1200, 1300, 1400 or 1500 or more biomolecules are detected according to the present method.

As used herein a “genetically modifying agent” is a substance that alters the genetic sequence of a cell following exposure to the cell, resulting in an agent-mediated nucleic acid sequence. In embodiments, the genetically modifying agent is a small molecule, protein, pathogen (e.g., virus or bacterium), toxin, oligonucleotide, or antigen. In embodiments, the genetically modifying agent is a virus (e.g., influenza) and the agent-mediated nucleic acid sequence is the nucleic acid sequence that develops within a T-cell upon cellular exposure and contact with the virus. In embodiments, the genetically modifying agent modulates the expression of a nucleic acid sequence in a cell relative to a control (e.g., the absence of the genetically modifying agent).

The term “synthetic agent” as used herein refers to a modified protein or nucleic acid such as those constructed by synthetic methods. In embodiments, a synthetic target is artificial or engineered, or derived from or contains an artificial or engineered protein or nucleic acid (e.g., non-natural or not wild type). For example, a polynucleotide that is inserted or removed such that it is not associated with nucleotide sequences that normally flank the polynucleotide as it is found in nature is a synthetic target polynucleotide.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2-dimensional representation of a 3-dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “tissue section” refers to a piece of tissue that has been obtained from a subject, optionally fixed, and attached to a surface, e.g., a microscope slide.

As used herein, “probe oligonucleotide” refers to the oligonucleotide attached, conjugated, or otherwise linked to a probe (e.g., a proximity probe or probe described herein). In embodiments, the probe oligonucleotide is a single-stranded oligonucleotide. In embodiments, the probe oligonucleotide is partially double-stranded. In embodiments, the 3′ end of the probe oligonucleotide is single-stranded. In embodiments, the proximity probe is covalently linked via a linker to the probe oligonucleotide. In embodiments, the linker includes one or more cleavable sites. In embodiments, the probe oligonucleotide includes the linker (i.e., the probe linker) covalently attached to the proximity probe.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

It will be appreciated that a barcode sequence and a complement of the barcode sequence, as described in the methods and compositions herein, are equivalent, in that if one sequence is known then the other sequence may be deduced and/or inferred.

As used herein, the term “complex” is intended to mean an element that includes two or more elements with different functional properties than one another. As described herein, a “protein-probe complex” refers to a complex including one or more proteins (e.g., a protein of interest) bound to a first probe and a second probe (see, e.g., FIG. 1E), which forms following the binding of the first probe and the second probe to one of more proteins of interest. In embodiments, “protein-probe complex” includes more than one protein bound to a first probe and a second probe. In embodiments, “protein-probe complex” includes two, three, or four proteins indirectly bound to a first probe described herein and a second probe described herein (see, e.g., FIG. 1D, 6A, 6B, or 6C). As described herein, a “linked complex” refers to a complex including a coupling oligonucleotide (see, e.g., FIG. 1A) and a protein-probe complex described herein (e.g., depicted as (9) in FIG. 1D); the linked complex forms as a result of hybridizing a first sequence of the coupling oligonucleotide to a first oligonucleotide attached to a first probe described herein and hybridizing a second sequence of the coupling oligonucleotide to a second oligonucleotide attached to a second probe described herein. As described herein, a “linked-bridge complex” refers to a complex including the linked complex described herein with a bridge oligonucleotide (see, e.g., FIG. 1B); the linked-bridge complex forms as a result of hybridizing a first sequence of the bridge oligonucleotide to the first oligonucleotide attached to the first probe described herein and hybridizing a second sequence of the bridge oligonucleotide to a second oligonucleotide attached to the second probe described herein.

The term “coupling oligonucleotide”, or “coupling oligo”, as used herein, refer to a nucleic acid that can anneal to two or more separate oligonucleotides when the two or more separate oligonucleotides are in close proximity to each other. In embodiments, a coupling oligonucleotide is a padlock probe (PLP) that includes a first sequence capable of hybridizing with the first oligonucleotide attached to a first probe described herein and a second sequence capable of hybridizing with the second oligonucleotide attached to a second probe described herein.

As used herein, “lectin” is intended to mean a protein that selectively binds a particular sugar or sugars, and as such does not bind any other sugars. “Monovalent” lectins may bind a single sugar at a given time, while “divalent” lectins may bind two sugars at once, and “multivalent” lectins may bind two or more sugars at once. Lectins may be naturally occurring, or non-naturally occurring. Naturally occurring lectins may include plant lectins and animal lectins.

### II. Methods

In an aspect is provided a method of forming a circular oligonucleotide. In embodiments, the method includes: (i) contacting a protein-probe complex with a coupling oligonucleotide, wherein the protein-probe complex includes one or more proteins bound to a first probe and a second probe, the first probe includes a first specific binding molecule (e.g., an antibody) attached to a first oligonucleotide tag including a first barcode sequence, and the second probe includes a second specific binding molecule (e.g., an antibody) attached to a second oligonucleotide tag including a second barcode sequence, and hybridizing a first sequence of the coupling oligonucleotide to the first oligonucleotide tag and hybridizing a second sequence of the coupling oligonucleotide to the second oligonucleotide tag, thereby forming a linked complex including the protein-probe complex and the coupling oligonucleotide; (ii) contacting the linked complex with a bridge oligonucleotide and hybridizing a first sequence of the bridge oligonucleotide to the first oligonucleotide tag and hybridizing a second sequence of the bridge oligonucleotide to the second oligonucleotide tag, thereby forming a linked-bridge complex; and (iii) extending the second sequence of the coupling oligonucleotide with a polymerase to form a complement of the second barcode sequence; extending the second sequence of the bridge oligonucleotide with a polymerase to form a complement of the first barcode sequence; and ligating the complement of the second barcode sequence to the first sequence of the bridge oligonucleotide and ligating the complement of the first barcode sequence to form the circular oligonucleotide. In embodiments, the first oligonucleotide tag is the first oligonucleotide described herein. In embodiments, the second oligonucleotide tag is the second oligonucleotide described herein. In embodiments, the protein-probe complex includes one, two, or three proteins bound directly or indirectly to the first probe and second probe.

In an aspect is provided a method of forming a circular oligonucleotide in or on a cell or tissue. In embodiments, the method includes: (i) contacting a protein-probe complex with a coupling oligonucleotide, wherein the protein-probe complex includes a protein bound to a first probe and a second probe, the first probe includes a first specific binding molecule (e.g., an antibody) attached to a first oligonucleotide including a first barcode sequence, and the second probe includes a second specific binding molecule (e.g., an antibody) attached to a second oligonucleotide including a second barcode sequence, and hybridizing a first sequence of the coupling oligonucleotide to the first oligonucleotide and hybridizing a second sequence of the coupling oligonucleotide to the second oligonucleotide, thereby forming a linked complex including the protein-probe complex and the coupling oligonucleotide; (ii) contacting the linked complex with a bridge oligonucleotide and hybridizing a first sequence of the bridge oligonucleotide to the first oligonucleotide and hybridizing a second sequence of the bridge oligonucleotide to the second oligonucleotide, thereby forming a linked-bridge complex; and (iii) extending the second sequence of the coupling oligonucleotide with a polymerase to form a complement of the second barcode sequence; extending the second sequence of the bridge oligonucleotide with a polymerase to form a complement of the first barcode sequence; and ligating the complement of the second barcode sequence to the first sequence of the bridge oligonucleotide and ligating the complement of the first barcode sequence to form the circular oligonucleotide.

In an aspect is provided a method of forming a circular oligonucleotide (e.g., a circular oligonucleotide including two barcode sequences). In embodiments, the method includes detecting the circular oligonucleotide. In embodiments, the method includes amplifying the circular oligonucleotide. In embodiments, the method includes (i) contacting a protein-probe complex with a coupling oligonucleotide, wherein the protein-probe complex includes a protein bound to a first probe and a second probe, the first probe attached to a first oligonucleotide including a first barcode sequence and the second probe attached to a second oligonucleotide including a second barcode sequence, and hybridizing a first sequence of the coupling oligonucleotide to the first oligonucleotide and hybridizing a second sequence of the coupling oligonucleotide to the second oligonucleotide, thereby forming a linked complex including the protein-probe complex and the coupling oligonucleotide; (ii) contacting the linked complex with a bridge oligonucleotide and hybridizing a first sequence of the bridge oligonucleotide to the first oligonucleotide and hybridizing a second sequence of the bridge oligonucleotide to the second oligonucleotide, thereby forming a linked-bridge complex; and (iii) extending the second sequence of the coupling oligonucleotide with a polymerase to form a complement of the second barcode sequence; extending the second sequence of the bridge oligonucleotide with a polymerase to form a complement of the first barcode sequence; and ligating the complement of the second barcode sequence to the first sequence of the bridge oligonucleotide and ligating the complement of the first barcode sequence to form the circular oligonucleotide. In embodiments, the method includes detecting a first molecule and a second molecule (e.g., a first protein and a second protein) based on at least the detection of the first and second barcodes.

In embodiments, the method includes forming the protein-probe complex (e.g., prior to step (i) of the aforementioned method). For example, forming the protein-probe complex may include binding the first probe to the protein, wherein the first probe includes a first specific binding reagent attached to the first oligonucleotide, and binding the second probe to the protein, wherein the second probe includes a second specific binding reagent attached to the second oligonucleotide. In embodiments, the protein may be within a cell, or on a cell (e.g., a cell-surface protein). In embodiments, the protein may be within a cell (e.g., a signal transduction protein or transcription factor). In embodiments, the protein may be outside a cell and in a tissue sample. An example of a protein-probe complex is illustrated in FIG. 1D, wherein a first probe (3 of FIG. 1C) is bound to a protein, wherein the probe is attached to an oligonucleotide tag (5 of FIG. 1C). In embodiments, forming the protein-probe complex (9 of FIG. 1D) is performed under suitable incubation conditions. Factors that may impact the formation of protein-probe complexes include, but are not limited to, incubation time, temperature, and probe concentration.

In some embodiments, samples (e.g., a sample including one or more cells or tissues, as described herein) may be pretreated to facilitate the formation of the protein-probe complexes. Pretreatments for increasing the availability of protein targets for interaction with specific detection reagents in situ (e.g., “antigen retrieval”) are known in the art, as exemplified by Shi et al. 1997, J. Histochem Cytochem, 45 (3): 327. In some embodiments, antigen retrieval may be achieved using protease-induced epitope retrieval (PIER), and may employ enzymes such as proteinase K, pepsin, trypsin, protease, and any subtypes thereof, in an appropriate buffer to restore the epitope for antibody binding. In some embodiments, antigen retrieval may be achieved using heat-induced epitope retrieval (HIER), and may employ heat to reverse some cross-links and allow the restoration of epitopes. In some embodiments, citrate buffers, Tris, and EDTA base may be employed as exemplary heat-induced reagents in appropriately pH stabilized manner (e.g., 10 mM sodium citrate, 6.0 pH; 1 mM EDTA, pH 8.0; 10 mM Tris base, 1 mM EDTA solution, 0.05% Tween 20, pH 9.0). Detergents (e.g., Tween 20) may be added to the HIER buffer to increase the epitope retrieval. In certain aspects, many proprietary formulations are available for the PIER or HIER mediate antigen retrieval.

In some embodiments, the sample may be treated with a blocking solution, prior to introduction of the probes to the sample, in order to reduce the likelihood of unspecific binding of the probes. Typically, depending on the tissue type and the method of antigen detection, endogenous biotin or enzymes may need to be blocked or quenched, respectively, prior to antibody staining. In some embodiments, samples are incubated with a “blocking buffer” that blocks reactive sites to which the probes may otherwise bind. In embodiments, the blocking buffer may include normal serum (i.e., goat serum). In embodiments, the blocking buffer may include non-fat dry milk. In embodiments, the blocking buffer may include FBS (fetal bovine serum). In embodiments, the blocking buffer may include BSA (bovine serum albumin). In embodiments, the blocking buffer may include gelatin. In embodiments, any number of commercial blocking buffers, each with proprietary formulations, may be used. There are many commercial blocking buffers available that are known in the art. In some embodiments, the sample may incubated with the blocking buffer for about 5 minutes to about 1 hour, about 5 minutes to about 40 minutes, about 5 minutes to about 30 minutes, about 5 minutes to about 20 minutes, or about 5 minutes to about 10 minutes. In some embodiments, the sample may incubated with the blocking buffer at room temperature. In some embodiments, the sample may incubated with the blocking buffer at a temperature of about 4° C. to about 35° C., about 4° C. to about 25° C., about 4° C. to about 20° C., about 4° C. to about 10° C., about 10° C. to about 25° C., about 10° C. to about 20° C., about 10° C. to about 15° C., about 35° C. to about 50° C., about 35° C. to about 45° C., about 35° C. to about 40° C., about 40° C. to about 50° C., about 40° C. to about 45° C., or about 45° C. to about 50° C.

Methods for optimizing probe concentration to minimize background staining and to maximize probe-target complex formation in situ are known in the art. Such methods include, but are not limited to, experimenting with serial dilutions of the concentrated antibody. For example, a common approach includes starting with the dilution recommended by the manufacturer with one dilution concentration above and one dilution concentration below. For example, if the recommended dilution is 1:400, then testing serial dilutions of 1:200, 1:400, and 1:800 may show optimal signal to background staining on one of the dilutions.

Methods for optimizing incubation time and/or incubation temperature to minimize background staining and to maximize probe-target complex formation in situ are known in the art. In some embodiments, the sample may incubated with the probe for about 60 minutes to about 120 minutes, about 60 minutes to about 110 minutes, about 60 minutes to about 100 minutes, about 60 minutes to about 90 minutes, or about 60 minutes to about 80 minutes. In some embodiments, the sample may incubated with the probe overnight. In some embodiments, the sample may incubated with the probe at room temperature. In some embodiments, the sample may incubated with the probe at a temperature of about 4° C. to about 35° C., about 4° C. to about 25° C., about 4° C. to about 20° C., about 4° C. to about 10° C., about 10° C. to about 25° C., about 10° C. to about 20° C., about 10° C. to about 15° C., about 35° C. to about 50° C., about 35° C. to about 45° C., about 35° C. to about 40° C., about 40° C. to about 50° C., about 40° C. to about 45° C., or about 45° C. to about 50° C.

In some embodiments, the sample may be washed, e.g., with a washing buffer, after probe incubation is completed. In some embodiments, the sample may be washed in around 3 distinct changes of washing buffer. In some embodiments, the washing buffer may contain around 0.1% TBS-Tween. In some embodiments, the sample may be washed using a different reaction container for each wash. In some embodiments, the sample is washed for a total of about 5 minutes to about 20 minutes, about 9 minutes to about 20 minutes, about 10 minutes to about 20 minutes, about 11 minutes to about 20 minutes, about 12 minutes to about 20 minutes, about 13 minutes to about 20 minutes, or about 15 minutes to about 20 minutes.

In embodiments, controls may be included. For example, a tissue known to express the antigen may be used as a positive control. In embodiments, a tissue known not to express the antigen may be used as a negative control. In embodiments, a tissue may be probed in the same way, albeit with the omission of the target-specific probe, as a negative control.

In embodiments, the method includes (i) binding a first probe to a first protein and binding a second probe to a second, different, protein, wherein the first probe includes a specific binding reagent attached to a first oligonucleotide including a first barcode sequence, and the second probe includes a specific binding reagent attached to a second oligonucleotide including a second barcode sequence, thereby forming a protein-probe complex; (ii) contacting the protein-probe complex with a coupling oligonucleotide and hybridizing a first sequence of the coupling oligonucleotide to the first oligonucleotide and hybridizing a second sequence of the coupling oligonucleotide to the second oligonucleotide, thereby forming a linked complex including the protein-probe complex and the coupling oligonucleotide; (iii) contacting the linked complex with a bridge oligonucleotide and hybridizing a first sequence of the bridge oligonucleotide to the first oligonucleotide and hybridizing a second sequence of the bridge oligonucleotide to the second oligonucleotide, thereby forming a linked-bridge complex; and (iv) extending the second sequence of the coupling oligonucleotide with a polymerase to form a complement of the second barcode sequence; extending the second sequence of the bridge oligonucleotide with a polymerase to form a complement of the first barcode sequence; and ligating the complement of the second barcode sequence to the first sequence of the bridge oligonucleotide and ligating the complement of the first barcode sequence to form the circular oligonucleotide.

In embodiments, the first protein and the second protein are different proteins (e.g., a CD2 protein and a CD58 protein). In embodiments, the first protein and the second protein are the same protein. In embodiments, the first probe and the second probe contact the same protein (e.g., the first protein and the second protein are different epitopes on the same protein). In embodiments, the first probe and the second probe contact different proteins. In embodiments, the first probe and the second probe contact the same protein. In embodiments, the second probe binds to an agent (e.g., a small molecule or analyte). In embodiments, the agent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polykctide, a polylysine, polyethylenciminc, dicthylaminocthyl (DEAE)-dextran, cholesterol, or a sterol moiety. In embodiments, the first protein and the second protein are both bound to a third protein. In embodiments, the first protein is bound to a third protein and the second protein is bound to a fourth protein.

In embodiments, the protein-probe complex is in or on a cell or tissue. In embodiments, the cell or tissue is fixed to a solid support. In embodiments, the cell is attached to a substrate. In embodiments, the cell is attached to the substrate via a bioconjugate reactive moiety. In embodiments, the protein-probe complex is on a cell or tissue sample. In embodiments, the cell or tissue sample is cleared (e.g., digested) of proteins, lipids, or proteins and lipids. In embodiments, the cell or tissue sample is processed according to a known technique in the art, for example CLARITY (Chung K., et al. Nature 497, 332-337 (2013)), PACT-PARS (Yang B., et al. Cell 158, 945-958 (2014).), CUBIC (Susaki E. A. et al. Cell 157, 726-739 (2014)., 18), ScaleS (Hama H., et al. Nat. Neurosci. 18, 1518-1529 (2015)), OPTIClear (Lai H. M., et al. Nat. Commun. 9, 1066 (2018)), Cₑ3D (Li W., et al. Proc. Natl. Acad. Sci. U.S.A. 114, E7321-E7330 (2017)), BABB (Dodt H.U. et al. Nat. Methods 4, 331-336 (2007)), iDISCO (Renier N., et al. Cell 159, 896-910 (2014)), uDISCO (Pan C., et al. Nat. Methods 13, 859-867 (2016)), FluoClearBABB (Schwarz M. K., et al. PLOS ONE 10, c0124650 (2015)), Ethanol-ECi (Klingberg A., et al. J. Am. Soc. Nephrol. 28, 452-459 (2017)), and PEGASOS (Jing D. et al. Cell Res. 28, 803-818 (2018)).

In embodiments, the cell forms part of a tissue in situ. In embodiments, the cell is an isolated single cell. In embodiments, the cell is a prokaryotic cell. In embodiments, the cell is a eukaryotic cell. In embodiments, the cell is a bacterial cell, a fungal cell, a plant cell, or a mammalian cell. In embodiments, the cell is a stem cell. In embodiments, the stem cell is an embryonic stem cell, a tissue-specific stem cell, a mesenchymal stem cell, or an induced pluripotent stem cell. In embodiments, the cell is an endothelial cell, muscle cell, myocardial, smooth muscle cell, skeletal muscle cell, mesenchymal cell, epithelial cell; hematopoietic cell, such as lymphocytes, including T cell, e.g., (Th 1 T cell, Th2 T cell, Th0 T cell, cytotoxic T cell); B cell, pre-B cell; monocytes; dendritic cell; neutrophils; or a macrophage. In embodiments, the cell is a stem cell, an immune cell, a cancer cell, a viral-host cell, or a cell that selectively binds to a desired target. In embodiments, the cell includes a T cell receptor gene sequence, a B cell receptor gene sequence, or an immunoglobulin gene sequence. In embodiments, the cell includes a Toll-like receptor (TLR) gene sequence. In embodiments, the cell includes a gene sequence corresponding to an immunoglobulin light chain polypeptide and a gene sequence corresponding to an immunoglobulin heavy chain polypeptide. In embodiments, the cell is a genetically modified cell.

In embodiments, the cell is a viral-host cell. A “viral-host cell” is used in accordance with its ordinary meaning in virology and refers to a cell that is infected with a viral genome (e.g., viral DNA or viral RNA). The cell, prior to infection with a viral genome, can be any cell that is susceptible to viral entry. In embodiments, the viral-host cell is a lytic viral-host cell. In embodiments, the viral-host cell is capable of producing viral protein. In embodiments, the viral-host cell is a lysogenic viral-host cell. In embodiments, the cell is a viral-host cell including a viral nucleic acid sequence, wherein the viral nucleic acid sequence is from a Hepadnaviridae, Adenoviridae, Herpesviridae, Poxviridae, Parvoviridae, Reoviridae, Coronaviridae, Retroviridae virus.

In embodiments, the cell is an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. Examples of such cells include, but are not limited to, cell lines derived from hematopoietic cells, and from the following cell lines: Colo205, CCRF-CEM, HL-60, K562, MOLT-4, RPMI-8226, SR, HOP-92, NCI-H322M, and MALME-3M. Non-limiting examples of adherent cells include DU145 (prostate cancer) cells, H295R (adrenocortical cancer) cells, HeLa (cervical cancer) cells, KBM-7 (chronic myelogenous leukemia) cells, LNCaP (prostate cancer) cells, MCF-7 (breast cancer) cells, MDA-MB-468 (breast cancer) cells, PC3 (prostate cancer) cells, SaOS-2 (bone cancer) cells, SH-SY5Y (neuroblastoma, cloned from a myeloma) cells, T-47D (breast cancer) cells, THP-1 (acute myeloid leukemia) cells, U87 (glioblastoma) cells, National Cancer Institute's 60 cancer cell line panel (NCI60), vero (African green monkey Chlorocebus kidney epithelial cell line) cells, MC3T3 (embryonic calvarium) cells, GH3 (pituitary tumor) cells, PC12 (pheochromocytoma) cells, dog MDCK kidney epithelial cells, Xenopus A6 kidney epithelial cells, zebrafish AB9 cells, and Sf9 insect epithelial cells. In embodiments, the cell is a neuronal cell, an endothelial cell, epithelial cell, germ cell, plasma cell, a muscle cell, peripheral blood mononuclear cell (PBMC), a myocardial cell, or a retina cell.

In embodiments, the cell is bound to a known antigen. In embodiments, the cell is a cell that selectively binds to a desired target, wherein the target is an antibody, or antigen binding fragment, an aptamer, affimer, non-immunoglobulin scaffold, small molecule, or genetic modifying agent. In embodiments, the cell is a leukocyte (i.e., a white-blood cell). In embodiments, leukocyte is a granulocyte (neutrophil, eosinophil, or basophil), monocyte, or lymphocyte (T cells and B cells). In embodiments, the cell is a lymphocyte. In embodiments, the cell is a T cell, an NK cell, or a B cell. In embodiments, the cell is an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the plurality of target nucleic acids includes non-contiguous regions of a nucleic acid molecule. In embodiments, the non-contiguous regions include regions of a VDJ recombination of a B cell or T cell.

In embodiments, the cell is a cancer cell. In embodiments, the cancer is lung cancer, colorectal cancer, skin cancer, colon cancer, pancreatic cancer, breast cancer, cervical cancer, lymphoma, leukemia, or a cancer associated with aberrant K-Ras, aberrant APC, aberrant Smad4, aberrant p53, or aberrant TGFβ. In embodiments, the cancer cell includes a ERBB2, KRAS, TP53, PIK3CA, or FGFR2 gene. In embodiments, the cancer cell includes a HER2 gene. In embodiments, the cancer cell includes a cancer-associated gene (e.g., an oncogene associated with kinases and genes involved in DNA repair) or a cancer-associated biomarker. A “biomarker” is a substance that is associated with a particular characteristic, such as a disease or condition. A change in the levels of a biomarker may correlate with the risk or progression of a disease or with the susceptibility of the disease to a given treatment. In embodiments, the cancer is Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, or Uveal Melanoma. In embodiments, the cancer-associated gene is a nucleic acid sequence identified within The Cancer Genome Atlas Program, accessible at online at cancer.gov/tcga.

In embodiments, the cell in situ is obtained from a subject (e.g., human or animal tissue). Once obtained, the cell is placed in an artificial environment in plastic or glass containers supported with specialized medium containing essential nutrients and growth factors to support proliferation. In embodiments, the cell is permeabilized and immobilized to a solid support surface (e.g., a microplate). In embodiments, the cell is permeabilized and immobilized within a well of the microplate. In embodiments, the cell is immobilized to a solid support surface (e.g., a well or a slide). In embodiments, the surface includes a patterned surface (e.g., suitable for immobilization of a plurality of cells in an ordered pattern. In embodiments, a plurality of cells is immobilized in wells of a microplate that have a mean or median separation from one another of about 10⁻²⁰ μm. In embodiments, a plurality of cells is immobilized in wells of a microplate that have a mean or median separation from one another of about 10-20; 10-50; or 100 μm. In embodiments, a plurality of cells is arrayed on a substrate (e.g., a solid support). In embodiments, the solid support includes a functionalized glass surface or a functionalized plastic surface. In embodiments, the cell is attached to a multiwell container (e.g., a flow cell).

In embodiments, the cell is attached to the substrate (e.g., a solid support, such as a plastic or glass solid support) via a bioconjugate reactive linker. In embodiments, the cell is attached to the substrate via a specific binding reagent. In embodiments, the specific binding reagent includes an antibody, single-chain Fv fragment (scFv), antibody fragment-antigen binding (Fab), or an aptamer. In embodiments, the specific binding reagent includes an antibody, or antigen binding fragment, an aptamer, affimer, or non-immunoglobulin scaffold. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. Substrates may be prepared for selective capture of particular cells. For example, a substrate containing a plurality of bioconjugate reactive moieties or a plurality of specific binding reagents, optionally in an ordered pattern, contacts a plurality of cells. Only cells containing complementary bioconjugate reactive moieties or complementary specific binding reagents are capable of reacting, and thus adhering, to the substrate. In embodiments, the cell is immobilized to a substrate. Substrates can be two- or three-dimensional and can include a planar surface (e.g., a glass slide). A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites. In embodiments, the substrate includes a polymeric coating, optionally containing bioconjugate reactive moieties capable of affixing the sample. Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a sample. In embodiments, the substrate is not a flow cell. In embodiments, the substrate includes a polymer matrix material (e.g., polyacrylamide, cellulose, alginate, polyamide, cross-linked agarose, cross-linked dextran or cross-linked polyethylene glycol), which may be referred to herein as a “matrix”, “synthetic matrix”, “exogenous polymer” or “exogenous hydrogel”. In embodiments, a matrix may refer to the various components and organelles of a cell, for example, the cytoskeleton (e.g., actin and tubulin), endoplasmic reticulum, Golgi apparatus, vesicles, etc. In embodiments, the matrix is endogenous to a cell. In embodiments, the matrix is exogenous to a cell. In embodiments, the matrix includes both the intracellular and extracellular components of a cell. In embodiments, polynucleotide primers may be immobilized on a matrix including the various components and organelles of a cell. Immobilization of polynucleotide primers on a matrix of cellular components and organelles of a cell is accomplished as described herein, for example, through the interaction/reaction of complementary bioconjugate reactive moieties. In embodiments, the exogenous polymer may be a matrix or a network of extracellular components that act as a point of attachment (e.g., act as an anchor) for the cell to a substrate.

In embodiments, the methods are performed in situ on isolated cells or in tissue sections (alternatively referred to as a sample) that have been prepared according to methodologies known in the art. Methods for permeabilization and fixation of cells and tissue samples are known in the art, as exemplified by Cremer et al., The Nucleus: Volume 1: Nuclei and Subnuclear Components, R. Hancock (ed.) 2008; and Larsson et al., Nat. Methods (2010) 7:395-397, the content of each of which is incorporated herein by reference in its entirety. In embodiments, the cell is cleared (e.g., digested) of proteins, lipids, or proteins and lipids. In embodiments, the biological sample can be permeabilized using any of the methods described herein (e.g., using any of the detergents described herein, e.g., SDS and/or N-lauroylsarcosine sodium salt solution) before or after enzymatic treatment (e.g., treatment with any of the enzymes described herein, e.g., trypsin, proteases (e.g., pepsin and/or proteinase K)). In embodiments, the biological sample can be permeabilized by contacting the sample with a permeabilization solution. In some embodiments, the biological sample is permeabilized by exposing the sample to greater than about 1.0 w/v % (e.g., greater than about 2.0 w/v %, greater than about 3.0 w/v %, greater than about 4.0 w/v %, greater than about 5.0 w/v %, greater than about 6.0 w/v %, greater than about 7.0 w/v %, greater than about 8.0 w/v %, greater than about 9.0 w/v %, greater than about 10.0 w/v %, greater than about 11.0 w/v %, greater than about 12.0 w/v %, or greater than about 13.0 w/v %) sodium dodecyl sulfate (SDS) and/or N-lauroylsarcosine or N-lauroylsarcosine sodium salt. In some embodiments, the biological sample can be permeabilized by exposing the sample (e.g., for about 5 minutes to about 1 hour, about 5 minutes to about 40 minutes, about 5 minutes to about 30 minutes, about 5 minutes to about 20 minutes, or about 5 minutes to about 10 minutes) to about 1.0 w/v % to about 14.0 w/v % (e.g., about 2.0 w/v % to about 14.0 w/v %, about 2.0 w/v % to about 12.0 w/v %, about 2.0 w/v % to about 10.0 w/v %, about 4.0 w/v % to about 14.0 w/v %, about 4.0 w/v % to about 12.0 w/v %, about 4.0 w/v % to about 10.0 w/v %, about 6.0 w/v % to about 14.0 w/v %, about 6.0 w/v % to about 12.0 w/v %, about 6.0 w/v % to about 10.0 w/v %, about 8.0 w/v % to about 14.0 w/v %, about 8.0 w/v % to about 12.0 w/v %, about 8.0 w/v % to about 10.0 w/v %, about 10.0% w/v % to about 14.0 w/v %, about 10.0 w/v % to about 12.0 w/v %, or about 12.0 w/v % to about 14.0 w/v %) SDS and/or N-lauroylsarcosine salt solution and/or proteinase K (e.g., at a temperature of about 4° C. to about 35° C., about 4° C. to about 25° C., about 4° C. to about 20° C., about 4° C. to about 10° C., about 10° C. to about 25° C., about 10° C. to about 20° C., about 10° C. to about 15° C., about 35° C. to about 50° C., about 35° C. to about 45° C., about 35° C. to about 40° C., about 40° C. to about 50° C., about 40° C. to about 45° C., or about 45° C. to about 50° C.).

In embodiments, the cell is exposed to paraformaldehyde (i.e., by contacting the cell with paraformaldehyde). In embodiments, the cell is exposed to glutaraldehyde (i.e., by contacting the cell with glutaraldehyde). Any suitable permeabilization and fixation technologies can be used for making the cell available for the detection methods provided herein. In embodiments the method includes affixing single cells or tissues to a transparent substrate. Exemplary tissue includes those from skin tissue, muscle tissue, bone tissue, organ tissue and the like. In embodiments, the method includes immobilizing the cell in situ to a substrate and permeabilized for delivering probes, enzymes, nucleotides and other components required in the reactions. In embodiments, the cell includes many cells from a tissue section in which the original spatial relationships of the cells are retained. In embodiments, the cell in situ is within a Formalin-Fixed Paraffin-Embedded (FFPE) sample. In embodiments, the cell is subjected to paraffin removal methods, such as methods involving incubation with a hydrocarbon solvent, such as xylene or hexane, followed by two or more washes with decreasing concentrations of an alcohol, such as ethanol. The cell may be rehydrated in a buffer, such as PBS, TBS or MOPs. In embodiments, the FFPE sample is incubated with xylene and washed using ethanol to remove the embedding wax, followed by treatment with Proteinase K to permeabilized the tissue. In embodiments, the cell is fixed with a chemical fixing agent. In embodiments, the chemical fixing agent is formaldehyde or glutaraldehyde. In embodiments, the chemical fixing agent is glyoxal or dioxolane. In embodiments, the chemical fixing agent includes one or more of ethanol, methanol, 2-propanol, acetone, and glyoxal. In embodiments, the chemical fixing agent includes formalin, Greenfix®, Greenfix® Plus, UPM, CyMol®, HOPE®, CytoSkelFix™, F-Solv®, FincFIX®, RCL2/KINFix, UMFIX, Glyo-Fixx®, Histochoice®, or PAXgenc®. In embodiments, the cell is fixed within a synthetic three-dimensional matrix (e.g., polymeric material). In embodiments, the synthetic matrix includes polymeric-crosslinking material. In embodiments, the material includes polyacrylamide, poly-ethylene glycol (PEG), poly(acrylate-co-acrylic acid) (PAA), or Poly(N-isopropylacrylamide) (NIPAM). In embodiments, the sample can be a biological sample selected from the group consisting of a freshly isolated sample, a fixed sample, a frozen sample, an embedded sample, a processed sample, or a combination thereof.

In embodiments the cell is lysed to release nucleic acid or other materials from the cells. For example, the cells may be lysed using reagents (e.g., a surfactant such as Triton-X or SDS, an enzyme such as lysozyme, lysostaphin, zymolase, cellulase, mutanolysin, glycanases, proteases, mannase, proteinase K, etc.) or a physical lysing mechanism a physical condition (e.g., ultrasound, ultraviolet light, mechanical agitation, etc.). The cells may release, for instance, DNA, RNA, mRNA, proteins, or enzymes. The cells may arise from any suitable source. For instance, the cells may be any cells for which nucleic acid from the cells is desired to be studied or sequenced, etc., and may include one, or more than one, cell type. The cells may be for example, from a specific population of cells, such as from a certain organ or tissue (e.g., cardiac cells, immune cells, muscle cells, cancer cells, etc.), cells from a specific individual or species (e.g., human cells, mouse cells, bacteria, etc.), cells from different organisms, cells from a naturally occurring sample (e.g., pond water, soil, etc.), or the like. In some cases, the cells may be dissociated from tissue. In embodiments, the method does not include dissociating the cell from the tissue or the cellular microenvironment. In embodiments, the method does not include lysing the cell.

In embodiments, a permeabilization solution can contain additional reagents or a biological sample may be treated with additional reagents in order to optimize biological sample permeabilization. In some embodiments, an additional reagent is an RNA protectant. As used herein, the term “RNA protectant” typically refers to a reagent that protects RNA from RNA nucleases (e.g., RNases). Any appropriate RNA protectant that protects RNA from degradation can be used. A non-limiting example of an RNA protectant includes organic solvents (e.g., at least 60%, 65%, 70%, 75%, 80%, 85%, 90%, or 95% v/v organic solvent), which includes ethanol, methanol, propan-2-ol, acetone, trichloroacetic acid, propanol, polyethylene glycol, acetic acid, or a combination thereof. In embodiments, the RNA protectant includes ethanol, methanol and/or propan-2-ol, or a combination thereof. In embodiments, the RNA protectant includes RNAlater ICE (ThermoFisher Scientific). In embodiments, the RNA protectant includes a salt. The salt may include ammonium sulfate, ammonium bisulfate, ammonium chloride, ammonium acetate, cesium sulfate, cadmium sulfate, cesium iron (II) sulfate, chromium (III) sulfate, cobalt (II) sulfate, copper (II) sulfate, lithium chloride, lithium acetate, lithium sulfate, magnesium sulfate, magnesium chloride, manganese sulfate, manganese chloride, potassium chloride, potassium sulfate, sodium chloride, sodium acetate, sodium sulfate, zinc chloride, zinc acetate and zinc sulfate. In some embodiments, the biological sample is treated with one or more RNA protectants before, contemporaneously with, or after permeabilization.

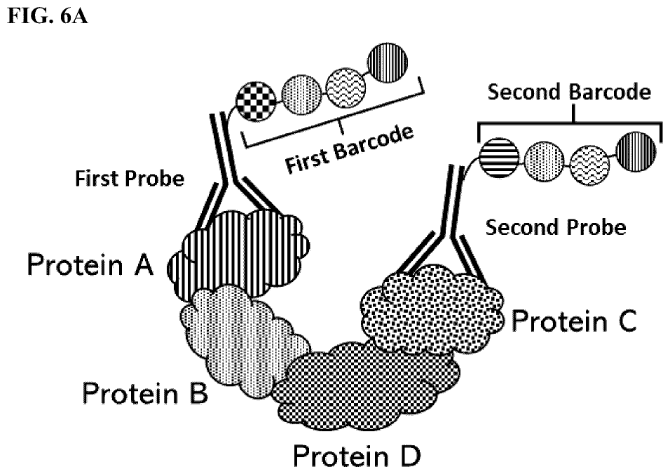

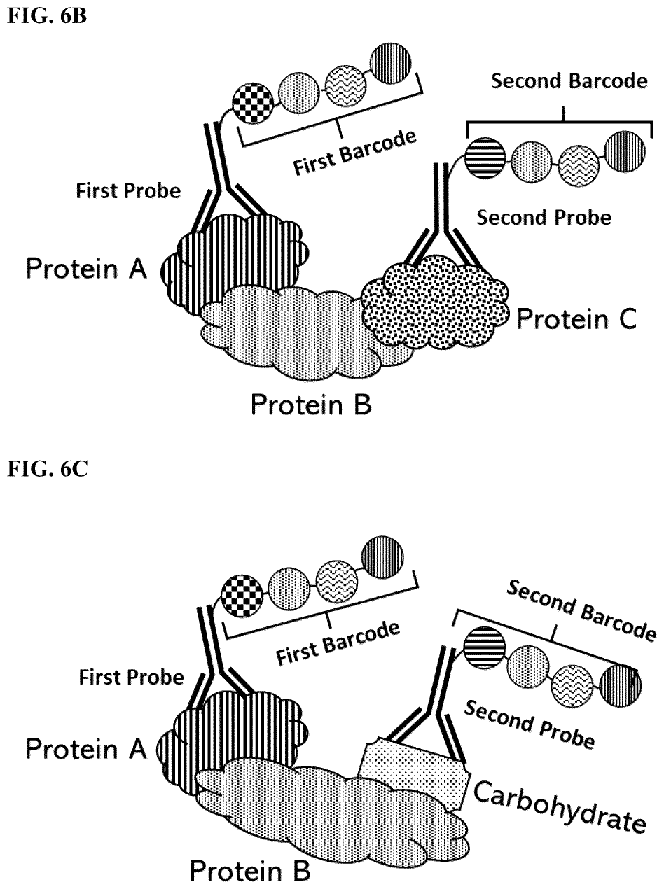

In embodiments, the second probe binds to a carbohydrate, which is attached to the protein. In embodiments, the probe binds to a glycoprotein at the surface of the cell or tissue. In embodiments, the probe binds to a glycolipid at the surface of the cell or tissue (e.g., as illustrated in FIG. 6C). Post-translational modifications (PTMs) like glycosylation and phosphorylation play crucial roles in regulating protein function and cellular processes. Glycosylation, for example, can affect protein stability, folding, localization, and recognition by other molecules. By detecting and studying these modifications, scientists can gain insights into protein structure, function, and signaling pathways, helping us understand cellular processes, diseases, and develop targeted therapies.

In embodiments, the second probe binds to a post-translational modification (e.g., phosphorylation analyte, methylation analyte, nitrosylation analyte, acetylation analyte, or glycosylation analyte). For example, phosphorylation is the addition of a phosphate group to a protein, such as phosphorylation of a serine residue in the protein kinase AMPK. In embodiments, the second probe binds to a phosphorylated amino acid. In embodiments, the second probe binds to a methylated amino acid. Methylation involves the addition of a methyl group to a protein. One example is the methylation of histone proteins, which can regulate gene expression by acting as a signal for other proteins to bind and modify chromatin structure. In embodiments, the second probe binds to a nitrosylated amino acid. Nitrosylation is the addition of a nitric oxide group to a protein. In cardiovascular disease, the nitrosylation of a specific cysteine residue in hemoglobin can affect its oxygen-carrying capacity. In embodiments, the second probe binds to an aceylated amino acid. Acetylation is the addition of an acetyl group to a protein. Acetylation of histone tails can alter the chromatin structure, influencing gene expression. For instance, acetylation of histone H3 lysine 9 (H3K9) is associated with transcriptional activation. In embodiments, the second probe binds to an glycosylated amino acid. Glycosylation refers to the addition of a sugar molecule (oligosaccharide) to a protein. One example is N-linked glycosylation, where sugars are added to a specific asparagine residue. Glycosylation of the glycoprotein erythropoietin is important for its stability and secretion.

In embodiments, the specific binding reagent includes the first specific binding reagent as described herein. In embodiments, the specific binding reagent includes the second specific binding reagent as described herein. In embodiments, the specific binding reagent further includes a third specific binding reagent, a fourth specific binding reagent, a fifth specific binding reagent, a sixth specific binding reagent, a seventh specific binding reagent, a eighth specific binding reagent, or additional specific binding reagents.

In embodiments, the specific binding reagent is capable of binding to a cluster of differentiation (CD) marker, integrin, selectin, cadherin, cytokine receptor, chemokine receptor, Toll-like receptor (TLR), ion channel, transmembrane protein, lipoprotein, glycoprotein, cell surface protein, transport protein, or transcription factor. In embodiments, the specific binding agent is capable of binding an intracellular organelle. In embodiments, the intracellular organelle includes actin, centrosomes and centrioles, chloroplasts (in plant cells and some protists), cytoskeleton, endoplasmic reticulum, endosome, golgi apparatus, intermediate filaments, lysosome, microfilaments, microtubules, mitochondria, nuclear envelope, nuclear pores, nucleoid, nucleolus, nucleus, peroxisome, phosphatidylserine, plasma membrane, ribosomes, rough endoplasmic reticulum, smooth endoplasmic reticulum, transferrin receptor, transport vesicles, and/or vacuoles. In embodiments, the biomolecule specific binding agent is capable of binding to a biomolecule in the mitogen-activated protein kinase (MAPK) pathway, PI3K/AKT/mTOR pathway, Wnt/β-catenin pathway, intrinsic (mitochondrial) pathway, extrinsic (death receptor) pathway, caspase cascade, Notch signaling pathway, hedgehog signaling pathway, TGF-β (transforming growth factor Beta) pathway, JAK/STAT pathway, G-protein coupled receptor (GPCR) pathway, calcium signaling pathway, glycolysis, citric acid cycle (Krebs Cycle), oxidative phosphorylation, lipid metabolism pathway, amino acid metabolism, Toll-like receptor (TLR) pathway, NF-κB signaling pathway, complement pathway, nucleotide excision repair (NER), base excision repair (BER), mismatch repair (MMR), cyclin-dependent kinase (CDK) pathway, Rb (retinoblastoma) pathway, p53 pathway, unfolded protein response (UPR), heat shock response pathway, oxidative stress pathway, BMP (bone morphogenetic protein) pathway, FGF (fibroblast growth factor) pathway, Sonic Hedgehog pathway, neurotrophin signaling pathway, synaptic transmission pathway, axon guidance pathways, insulin signaling pathway, thyroid hormone pathway, steroid hormone pathway, VEGF (vascular endothelial growth factor) pathway, DNA methylation pathway, histone modification pathway, or angiogenesis. In embodiments, the biomolecule specific binding agent is capable of binding to a biomolecule on the surface of or in a B cell, Mature B Cell, Follicular B cell, Marginal Zone B cell, Short lived plasma cell, Memory B cell, Long lived plasma cell, BI cell, Breg, Germinal Center B cell, Macrophage, Monocyte, M1 macrophage, M2 macrophage, Dendritic Cell, Plasmacytoid dendritic cell, Monocyte-derived dendritic cell, T cell, T Follicular Helper, Th1, Th2, Th9, Th17, Th22, Treg, platelet (activated), platelet (rested), natural killer cell, neutrophil, basophil, cosinophil, mast cell, astrocyte, neuron, glial cell, lymphocyte, myeloid cell, granulocytes, neural cells, stem cells, endothelial cells, epithelial cells, mesenchymal stem cell, hematopoietic stem cell, embryonic stem, stromal cell, erythrocyte, fibroblast, or apoptotic cell.

In embodiments, the first specific binding reagent is an antibody, single-chain Fv fragment (scFv), antibody fragment-antigen binding (Fab), lectin, affimer, or an aptamer. In embodiments, the second specific binding reagent is an antibody, single-chain Fv fragment (scFv), antibody fragment-antigen binding (Fab), lectin, affimer, or an aptamer. In embodiments, the first specific binding reagent is an antibody and the second specific binding reagent is an antibody. The selection of the specific binding reagent depends on the intended target biomolecule. In embodiments, the specific binding reagent binds to a target molecule having a K_{D} is less than 10⁻⁶ M, less than 10⁻⁷M, less than 10⁻⁸ M, less than 10⁻⁹ M, less than 10⁻⁹ M, less than 10⁻¹¹ M, or less than about 10⁻¹² M or less. The K_{D} represents the concentration of ligand (e.g., target molecule) at which half of the binding sites of the binding partner (e.g., specific binding reagent) are occupied. A lower K_{D} indicates stronger binding affinity, as less ligand is required to achieve 50% occupancy. In embodiments, the K_{D} is 10⁻⁶ M to 10⁻¹² M. In embodiments, the K_{D} is 10⁻⁸ M to 10⁻¹² M. In embodiments, the K_{D} is 10⁻⁹M to 10⁻¹² M. In embodiments, the K_{D} is 10⁻¹⁰ M to 10⁻¹² M. In embodiments, the K_{D} is 10⁻⁸ M to 10⁻¹⁰ M. Non-limited examples of target biomolecules and specific binding reagents is provided in the Target Table:

Target Table:

| Target Biomolecule | Specific Binding Reagent |
|---|---|
| Protein | Antibodies, aptamers, RNAs, modified bases |
| Carbohydrate | Lectins |
| Peptide | Antibodies or aptamers |
| Small molecule(s) | Proteins or aptamers |
| Lipids | Proteins or aptamers |
| Biotinylated biomolecule | Avidin (e.g., streptavidin) |

In embodiments, each probe is an antibody, an antibody fragment, an affimer, an aptamer, or a nucleic acid. The antibodies used for the probes may be polyclonal or monoclonal antibodies, or fragments of antibodies. Further, the antibodies forming the protein-probe complex may have the same binding specificity or differ in their binding specificities. Further contemplated herein is the use of variations, e.g., that are described in WO2012/104261, which is incorporated herein by reference in its entirety. For example, the probes may each be linked to their respective antibody at the 5′ end, or one probe may be linked at the 5′ end and the other at the 3′ end.

A probe is defined herein as an entity including an analyte-binding domain specific for a biomolecule (e.g., a protein), and a nucleic acid domain (e.g., a probe oligonucleotide). By “specific for biomolecule” is meant that the biomolecule-binding domain specifically recognizes and binds a particular target biomolecule, i.e., it binds its target biomolecule with higher affinity than it binds to other biomolecules or moieties. In embodiments, the biomolecule-binding domain is an antibody, in particular a monoclonal antibody. Antibody fragments or derivatives of antibodies including the biomolecule-binding domain are also suitable for use as the biomolecule binding domain. Examples of such antibody fragments or derivatives include Fab, Fab′, F(ab′)2 and scFv molecules.

A Fab fragment consists of the antigen-binding domain of an antibody. An individual antibody may be seen to contain two Fab fragments, each consisting of a light chain and its conjoined N-terminal section of the heavy chain. Thus, a Fab fragment contains an entire light chain and the VH and CHI domains of the heavy chain to which it is bound. Fab fragments may be obtained by digesting an antibody with papain.

F(ab′)2 fragments consist of the two Fab fragments of an antibody, plus the hinge regions of the heavy domains, including the disulfide bonds linking the two heavy chains together. In other words, a F(ab′)2 fragment can be seen as two covalently joined Fab fragments. F(ab′)2 fragments may be obtained by digesting an antibody with pepsin. Reduction of F(ab′)2 fragments yields two Fab′ fragments, which can be seen as Fab fragments containing an additional sulfhydryl group which can be useful for conjugation of the fragment to other molecules. ScFv molecules are synthetic constructs produced by fusing together the variable domains of the light and heavy chains of an antibody. Typically, this fusion is achieved recombinantly, by engineering the antibody gene to produce a fusion protein which includes both the heavy and light chain variable domains.

The nucleic acid domain of a probe may be a DNA domain or an RNA domain. Preferably it is a DNA domain. In embodiments, the nucleic acid domains (e.g., probe oligonucleotide) of the probes are designed to hybridize to another oligonucleotide molecule. In embodiments the probe oligonucleotides of the probes are single-stranded. In other embodiments, the probe oligonucleotides of the probes are partially single-stranded, including both a single-stranded portion and a double-stranded portion.

In embodiments, each probe binds to a protein with a specific binding affinity (e.g., a specific dissociation constant K_{D}). In embodiments, the first probe binds to the first protein with a specific binding affinity (e.g., a specific dissociation constant K_{D}). In embodiments, the second probe binds to the second protein with a specific binding affinity (e.g., a specific dissociation constant K_{D}). The equilibrium dissociation constant, K_{D}, is a measure of the strength of an interaction between a biomolecule and its binding partner. In embodiments, the proximity probe binds to the first molecule with a K_{D} in the low micromolar (106) to nanomolar (10⁻⁷ to 10⁻⁹) range. In embodiments, the probe binds to the first molecule with a K_{D} in the low nanomolar range (10⁻⁹). In embodiments, the probe binds to the first molecule with a K_{D} in the picomolar (10⁻¹²) range. In embodiments, the probe binds to the first molecule with a K_{D} of at least 10⁻⁹ nM. In embodiments, the probe binds to the first molecule with a K_{D} of at least 10⁻¹² nM.

In embodiments, specific binding entails a binding affinity, typically expressed as a K_{D} (such as a K_{D} measured by surface plasmon resonance at an appropriate temperature, such as 37° C.). In embodiments, the K_{D} of a specific binding interaction is less than about 100 nM, 50 nM, 10 nM, 1 nM, 0.05 nM, or lower. In embodiments, the K_{D} of a specific binding interaction is about 0.01-100 nM, 0.1-50 nM, or 1-10 nM. In embodiments, the K_{D} of a specific binding interaction is less than 10 nM. The binding affinity of an antibody can be readily determined by one of ordinary skill in the art (for example, by Scatchard analysis). A variety of immunoassay formats can be used to select antibodies specifically immunoreactive with a particular antigen. For example, solid-phase ELISA immunoassays are routinely used to select monoclonal antibodies specifically immunoreactive with an analyte. See Harlow and Lane, ANTIBODIES: A LABORATORY MANUAL, Cold Springs Harbor Publications, New York, (1988) for a description of immunoassay formats and conditions that can be used to determine specific immunorcactivity. Typically, a specific or selective reaction will be at least twice background signal to noise and more typically more than 10 to 100 times greater than background.

In embodiments, the first oligonucleotide is attached to the first probe via a linker. In embodiments, the second oligonucleotide is attached to the second probe via a linker. The two components of the probe (e.g., a protein-binding domain, such as an antibody, and a probe oligonucleotide) are joined together either directly through a bond or indirectly through a linking group. Where linking groups are employed, such groups may be chosen to provide for covalent attachment of the probe oligonucleotide and biomolecule-binding domains through the linking group, as well as maintain the desired binding affinity of the biomolecule-binding domain for its target biomolecule. Linking groups of interest may vary widely depending on the biomolecule-binding domain. The linking group (i.e., the linker), when present, is in many embodiments biologically inert. A variety of linking groups are known to those of skill in the art and find use in the subject proximity probes. In embodiments, the linking group is at least between 50 Daltons to 1,000 Daltons, 1,000 Daltons to 10,000 Daltons, or 10,000 Daltons to 100,000 Daltons. In embodiments, the linking group is generally at least about 50 Daltons, 100 Daltons, 300 Daltons, 500 Daltons, 1000 Daltons, 2000 Daltons, 3000 Daltons, 6000 Daltons, 12,000 Daltons, 30,000 Daltons, or larger, for example up to 1,000,000 Daltons. In embodiments, the linker may contain a spacer. Generally, such linkers will include a spacer group terminated at either end with a reactive functionality capable of covalently bonding to the probe oligonucleotide or biomolecule-binding moieties. Spacer groups of interest may include aliphatic and unsaturated hydrocarbon chains, spacers containing heteroatoms such as oxygen (ethers such as polyethylene glycol) or nitrogen (polyamines), peptides, carbohydrates, cyclic or acyclic systems that may possibly contain heteroatoms. Spacer groups may also be comprised of ligands that bind to metals such that the presence of a metal ion coordinates two or more ligands to form a complex. Specific spacer elements include: 1,4-diaminohexane, xylylenediamine, terephthalic acid, 3,6-dioxaoctanedioic acid, ethylenediamine-N,N-diacetic acid, 1,1′-ethylenebis(5-oxo-3-pyrrolidinecarboxylic acid), 4,4′-ethylenedipiperidine. Potential reactive functionalities include nucleophilic functional groups (amines, alcohols, thiols, hydrazides), electrophilic functional groups (aldehydes, esters, vinyl ketones, epoxides, isocyanates, malcimides), functional groups capable of cycloaddition reactions, forming disulfide bonds, or binding to metals. Specific examples include primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, and malcimides.

In embodiments, the oligonucleotide is covalently attached to a protein-specific binding agent, wherein the protein-specific binding agent is an antibody, single-chain Fv fragment (scFv), affimer, aptamer, single-domain antibody (sdAb), or antibody fragment-antigen binding (Fab). In embodiments, the oligonucleotide is covalently attached to an antibody or single-domain antibody (sdAb). In embodiments, the oligonucleotide is covalently attached to an antibody. In embodiments, the oligonucleotide is covalently attached to a single-chain Fv fragment (scFv). In embodiments, the oligonucleotide is covalently attached to an affimer. In embodiments, the oligonucleotide is covalently attached to an aptamer. In embodiments, the oligonucleotide is covalently attached to a single-domain antibody (sdAb). In embodiments, the oligonucleotide is covalently attached to an antibody fragment-antigen binding (Fab). In embodiments, the oligonucleotide is covalently attached to an antibody or single-domain antibody (sdAb). The design and preparation of protein-specific binding agent oligonucleotide conjugates is known, for example various different binding moieties which may be used, the design of probe oligonucleotides, and the coupling of such oligonucleotides to the binding moieties to form the conjugates. The details and principles may be applied to the design of the probes for use in the methods described herein. For example, reference may be made to WO 2007/107743, U.S. Pat. Nos. 7,306,904 and 6,878,515 which are incorporated herein by reference.

In embodiments, the oligonucleotide (e.g., the first oligonucleotide and/or the second oligonucleotide) is attached to a specific binding agent (e.g., an antibody) via a linker (e.g., a bioconjugate linker). In embodiments, the oligonucleotide is attached to the protein-specific binding agent via a linker formed by reacting a first bioconjugate reactive moiety (e.g., the bioconjugate reactive moiety includes an amine moiety, aldehyde moiety, alkyne moiety, azide moiety, carboxylic acid moiety, dibenzocyclooctyne (DBCO) moiety, tetrazine moiety, epoxy moiety, isocyanate moiety, furan moiety, malcimide moiety, thiol moiety, or transcyclooctone (TCO) moiety) with a second bioconjugate reactive moiety). In embodiments, the oligonucleotide includes a barcode, wherein the barcode is a known sequence associated with the specific binding reagent. In embodiments, the barcode is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length.

In embodiments, specific binding entails a binding affinity, expressed as a K_{D} (such as a K_{D} measured by surface plasmon resonance at an appropriate temperature, such as 37° C.). In embodiments, the K_{D} of a specific binding interaction is less than about 100 nM, 50 nM, 10 nM, 1 nM, 0.05 nM, or lower. In embodiments, the K_{D} of a specific binding interaction is about 0.01-100 nM, 0.1-50 nM, or 1-10 nM. In embodiments, the K_{D} of a specific binding interaction is less than 10 nM. The binding affinity of an antibody can be readily determined by one of ordinary skill in the art (for example, by Scatchard analysis). A variety of immunoassay formats can be used to select antibodies specifically immunorcactive with a particular antigen. For example, solid-phase ELISA immunoassays are routinely used to select monoclonal antibodies specifically immunoreactive with an analyte. Sec Harlow and Lane, ANTIBODIES: A LABORATORY MANUAL, Cold Springs Harbor Publications, New York, (1988) for a description of immunoassay formats and conditions that can be used to determine specific immunoreactivity. Typically, a specific or selective reaction will be at least twice background signal to noise and more typically more than 10 to 100 times greater than background.

Protein-specific binding agents, Ab-O conjugates, and the like, collectively referred to as “proximity probes” as used herein, are generally used in pairs, and individually consist of an biomolecule-binding domain with specificity to the target analyte or biomolecule, and a nucleic acid domain linked, coupled, or conjugated thereto. For example, the biomolecule-binding domain can be for example a nucleic acid “aptamer” (Fredriksson et al (2002) Nat Biotech 20:473-477) or can be proteinaccous, such as a monoclonal or polyclonal antibody (Gullberg et al (2004) Proc Natl Acad Sci USA 101:8420-8424). The respective biomolecule-binding domains of each proximity probe pair may have specificity for different binding sites on the biomolecule, which biomolecule may consist of a single molecule or a complex of interacting molecules, or may have identical specificities, for example in the event that the target biomolecule exists as a multimer. When a proximity probe pair come into close proximity with each other, which will primarily occur when both are bound to their respective sites on the biomolecule molecule or biomolecule complex, the oligonucleotides are able to interact, directly or indirectly.

In embodiments, prior to extending the first oligonucleotide, the method includes binding the protein-specific binding agent to a target molecule in a cell or tissue. In embodiments, the targets are proteins or carbohydrates. In embodiments, the targets are proteins. In embodiments, the targets are carbohydrates. In embodiments, the targets are nucleic acid molecules (e.g., RNA or DNA). In embodiments when the target are proteins and/or carbohydrates, the method includes contacting the proteins with a specific binding reagent, wherein the specific binding reagent comprises an oligonucleotide barcode sequence. In embodiments, the specific binding reagent comprises an antibody, single-chain Fv fragment (scFv), antibody fragment-antigen binding (Fab), or an aptamer. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. In embodiments, the specific binding reagent interacts (e.g., contacts, or binds) with one or more specific binding reagents on the cell surface. Carbohydrate-specific antibodies are known in the art, see for example Kappler, K., Hennet, T. Genes Immun 21, 224-239 (2020).

In embodiments, the protein-specific binding agent is an antibody, single-chain Fv fragment (scFv), affimer, aptamer, single-domain antibody (sdAb), or antibody fragment-antigen binding (Fab). In embodiments, the protein-specific binding agent is an antibody. In embodiments, the protein-specific binding agent is a single-chain Fv fragment (scFv). In embodiments, the protein-specific binding agent is an affimer. In embodiments, the protein-specific binding agent is an aptamer. In embodiments, the protein-specific binding agent is a single-domain antibody (sdAb). In embodiments, the protein-specific binding agent is an antibody fragment-antigen binding (Fab).

In embodiments, the first oligonucleotide includes about 20 to about 30 nucleotides. In embodiments, the first oligonucleotide includes about 25 to about 50 nucleotides. In embodiments, the first oligonucleotide includes about 25 to about 40 nucleotides. In embodiments, the second oligonucleotide includes about 20 to about 30 nucleotides. In embodiments, the second oligonucleotide includes about 25 to about 50 nucleotides. In embodiments, the second oligonucleotide includes about 25 to about 40 nucleotides.

Specific linker groups that may find use in the subject proximity probes include heterofunctional compounds, such as azidobenzoyl hydrazide, N-[4-(p-azidosalicylamino)butyl]-3′-[2′-pyridyldithio] propionamid), bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, and succinimidyl-4-[N-maleimidomethyl|cyclohexane-1-carboxylate, 3-(2-pyridyldithio) propionic acid N-hydroxysuccinimide ester (SPDP), 4-(N-maleimidomethyl)-cyclohexane-1-carboxylic acid N-hydroxysuccinimide ester (SMCC), and the like.

In embodiments, the first oligonucleotide includes, from 5′ to 3′, a first coupling sequence, a first barcode sequence, and a first bridge sequence. In embodiments, the first coupling sequence is a sequence capable of hybridizing (e.g., is complementary to) to a complementary sequence on the universal padlock probe (PLP) oligonucleotide. In embodiments, the first coupling sequence is a sequence capable of hybridizing (e.g., is complementary to) to a complementary sequence on the coupling oligonucleotide described herein. In embodiments, the first bridge sequence is a sequence capable of hybridizing (e.g., is complementary to) to a complementary sequence on the bridge oligonucleotide. For example, as illustrated in FIGS. 1A-1E, the first oligonucleotide tag (5 of FIG. 1C) includes the coupling sequence, A, which is complementary to the 5′ arm sequence, A′, of the universal (PLP) oligonucleotide, and the bridge binding sequence, B, which is complementary to the 3′ tag binding sequence, B′, of the universal bridge oligonucleotide.

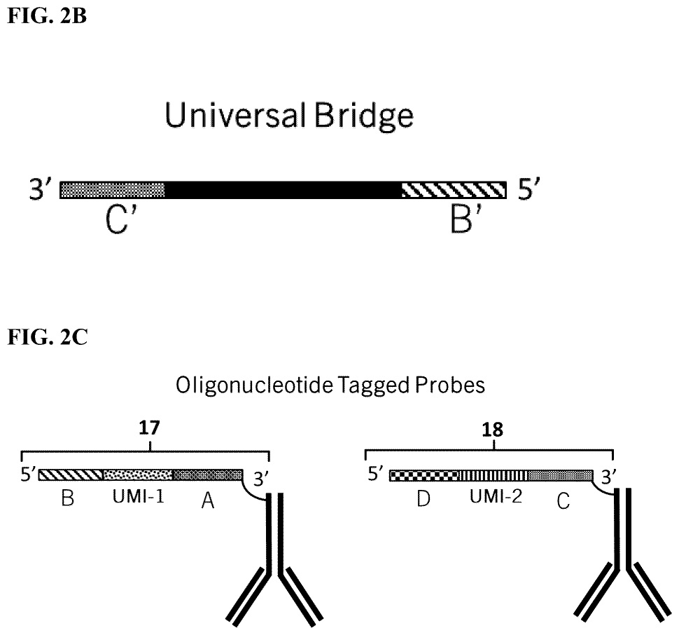

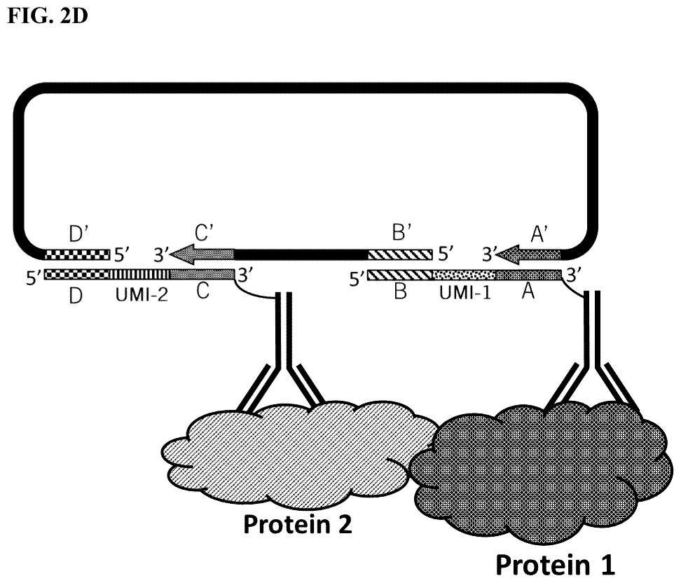

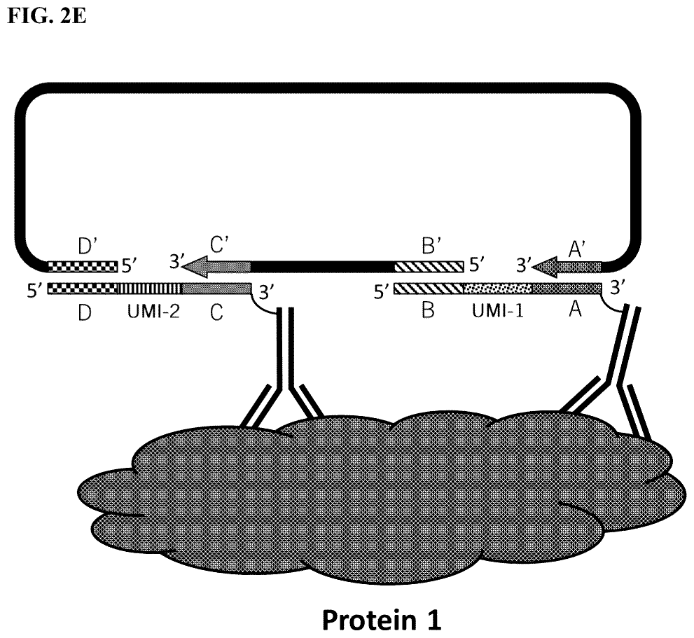

In embodiments, the first oligonucleotide tag includes, from 5′ to 3′, a first bridge binding sequence, a first barcode sequence, and a first coupling sequence. In embodiments, the first bridge binding sequence is a sequence capable of hybridizing to a complementary sequence on the universal bridge oligonucleotide. In embodiments, the first coupling sequence is a sequence capable of hybridizing to a complementary sequence on the universal padlock probe (PLP) oligonucleotide. For example, as illustrated in FIGS. 2A-2E, the first oligonucleotide (17) includes the bridge binding sequence, B, which is complementary to the 5′ tag binding sequence, B′, of the universal bridge oligonucleotide, and the coupling sequence, A, which is complementary to the 3′ arm sequence, A′, of the universal (PLP) oligonucleotide.

In embodiments, the second oligonucleotide tag includes, from 5′ to 3′, a second bridge binding sequence, a second barcode sequence, and a second coupling sequence. In embodiments, the second bridge binding sequence is a sequence capable of hybridizing (e.g., is complementary to) to a complementary sequence on the universal bridge oligonucleotide. In embodiments, the second coupling sequence is a sequence capable of hybridizing (e.g., is complementary to) to a complementary sequence on the coupling oligonucleotide. In embodiments, the second coupling sequence is a sequence capable of hybridizing (e.g., is complementary to) to a complementary sequence on the universal padlock probe (PLP) oligonucleotide. For example, as illustrated in FIGS. 1A-1E, the second oligonucleotide tag (8) includes the bridge binding sequence, C, which is complementary to the 5′ tag binding sequence, C′, of the universal bridge oligonucleotide, and the coupling sequence, D, which is complementary to the 3′ arm sequence, D′, of the universal padlock probe (PLP) oligonucleotide.

In embodiments, the second oligonucleotide includes, from 5′ to 3′, a second coupling sequence, a second barcode sequence, and a second bridge binding sequence. In embodiments, the second coupling sequence is a sequence capable of hybridizing (e.g., is complementary to) to a complementary sequence on the universal padlock probe (PLP) oligonucleotide. In embodiments, the second bridge binding sequence is a sequence capable of hybridizing (e.g., is complementary to) to a complementary sequence on the universal bridge oligonucleotide. For example, as illustrated in FIGS. 2A-2E, the second oligonucleotide tag (18) includes the coupling sequence, D, which is complementary to the 5′ arm sequence, D′, of the universal padlock probe (PLP) oligonucleotide, and the bridge binding sequence, C, which is complementary to the 3′ tag binding sequence, C′, of the universal bridge oligonucleotide.

In embodiments, the first barcode sequence and/or the second barcode sequence is 1 to 20 nucleotides. In embodiments, the barcode (i.e., the barcode sequence) is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. In embodiments, the barcode is at least about 1, 2, 3, 4, 5, 6, 7, 8, 9, 10, 15, 20, 25, 30, 35, 40, 45, 50 or more nucleotides in length. In embodiments, the barcode can be at most about 300, 200, 100, 90, 80, 70, 60, 50, 40, 30, 20, 15, 12, 10, 9, 8, 7, 6, 5, 4 or fewer or more nucleotides in length. In embodiments, the barcode includes between about 5 to about 8, about 5 to about 10, about 5 to about 15, about 5 to about 20, about 10 to about 150 nucleotides. In embodiments, the barcode includes between 5 to 8, 5 to 10, 5 to 15, 5 to 20, 10 to 150 nucleotides. In embodiments, the barcode is 10 nucleotides. In embodiments, the barcode may include a unique sequence (e.g., a barcode sequence) that gives the barcode its identifying functionality. The unique sequence may be random or non-random. Attachment of the barcode sequence (via binding of a probe conjugated to the barcode sequence) to a protein or nucleic acid of interest (i.e., the target) may associate the barcode sequence with the protein or nucleic acid of interest. The barcode may then be used to identify the protein or nucleic acid of interest during sequencing, even when other proteins or nucleic acids of interest (e.g., including different oligonucleotide barcodes) are present. In embodiments, the barcode consists only of a unique barcode sequence. In embodiments, the 5′ end of a barcoded oligonucleotide is phosphorylated. In embodiments, the barcode is known (i.e., the nucleic sequence is known before sequencing) and is sorted into a basis-set according to their Hamming distance. Oligonucleotide barcodes (e.g., barcode sequences included in an oligonucleotide) can be associated with a target of interest by knowing, a priori, the target of interest, such as a gene sequence or protein. The unique identifier sequence of the barcode can be a nucleic acid sequence which associates the oligonucleotide barcode with the protein(s) of interest.

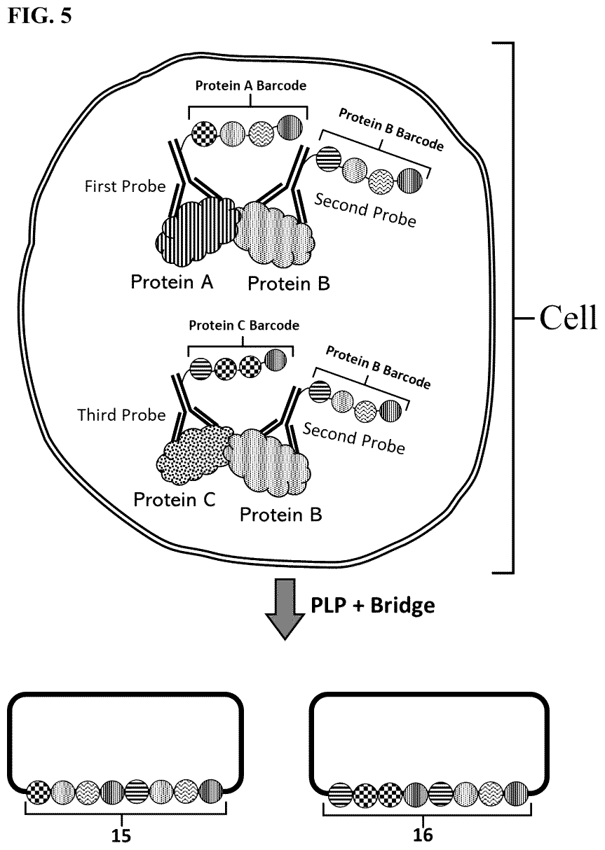

In embodiments, the method includes forming a unified barcode, wherein the unified barcode includes the first and second barcode sequences. For example, as illustrate in FIG. 5, the resulting association of the first barcode sequence and the second barcode sequence provides a circular polynucleotide including both barcode sequences. The resulting localized, circular RCA templates can then be amplified, and then sequenced thereby revealing the identities and of different interacting proteins. In embodiments, the unified barcode includes may have a length of 10 to 40, 10 to 30, 10 to 20, 25 to 50, 15 to 40, 15 to 30, 20 to 50, 20 to 40, or 20 to 30 nucleotides. It will be appreciated that a barcode sequence and a complement of the barcode sequence, as described in the methods and compositions herein, are equivalent, in that if one sequence is known then the other sequence may be deduced and/or inferred.

In embodiments, a barcode is a degenerate or partially-degenerate sequence, such that one or more nucleotides are selected at random from a set of two or more different nucleotides at one or more positions, with each of the different nucleotides selected at one or more positions represented in a pool of oligonucleotides including the degenerate or partially-degenerate sequence. The number of possible barcodes in a given set of barcodes will vary with the number of degenerate positions, and the number of bases permitted at each such position. For example, a barcode of five nucleotides (consecutive or non-consecutive), in which each position can be any of A, T, G, or C represents 54, or 1024 possible barcodes. In embodiments, certain barcode sequences may be excluded from a pool, such as barcodes in which every position is the same base. In embodiments, there are about, 102, 103 104, 105, 106, 107, 108, 109, or a number or a range between any two of these values, unique nucleotide barcode sequences. In embodiments, there are at least, or at most 102, 103 104, 105, 106, 107, 108, 109 unique barcode sequences. In embodiments, a barcode is about, 1, 2, 3, 4, 5, 10, 15, 20, 25, 30, 35, 40, 45, 50, or a number or a range between any two of these values, nucleotides in length. A barcode can be at least, or at most, 1, 2, 3, 4, 5, 10, 15, 20, 25, 30, 35, 40, 45, 50, 100, or 200 nucleotides in length.

In embodiments, the barcodes in the known set of barcodes have a specified Hamming distance. In embodiments, the Hamming distance is 4 to 15. In embodiments, the Hamming distance is 8 to 12. In embodiments, the Hamming distance is 10. In embodiments, the Hamming distance is 0 to 100. In embodiments, the Hamming distance is 0 to 15. In embodiments, the Hamming distance is 0 to 10. In embodiments, the Hamming distance is 1 to 10. In embodiments, the Hamming distance is 5 to 10. In embodiments, the Hamming distance is 1 to 100. In embodiments, the Hamming distance between any two barcode sequences of the set is at least 2, 3, 4, or 5. In embodiments, the Hamming distance between any two barcode sequences of the set is at least 3. In embodiments, the Hamming distance between any two barcode sequences of the set is at least 4.

In embodiments, the method further includes measuring an amount of one or more of the targets by counting the one or more associated barcodes. In embodiments, the method further includes counting the one or more associated barcodes in an optically resolved volume. In embodiments, the number of unique targets detected within an optically resolved volume of a sample is about 3, 10, 30, 50, or 100. In embodiments, the number of unique targets detected within an optically resolved volume of a sample is about 1 to 10. In embodiments, the number of unique targets detected within an optically resolved volume of a sample is about 5 to 10. In embodiments, the number of unique targets detected within an optically resolved volume of a sample is about 1 to 5. In embodiments, the number of unique targets detected within an optically resolved volume of a sample is at least 3, 10, 30, 50, or 100. In embodiments, the number of unique targets detected within an optically resolved volume of a sample is less than 3, 10, 30, 50, or 100. In embodiments, the number of unique targets detected within an optically resolved volume of a sample is about 1, 2, 3, 4, 5, 6, 7, 8, 9, 10, 20, 50, 100, 500, 1,000, 5,000, 10,000, or 200,000. In embodiments, the methods allow for detection of a single target of interest. In embodiments, the methods allow for multiplex detection of a plurality of targets of interest. The use of oligonucleotide barcodes with unique identifier sequences as described herein allows for simultaneous detection of 2, 3, 4, 5, 6, 7, 8, 9, 10, 20, 30, 40, 50, 60, 70, 80, 90, 100, 200, 300, 400, 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000 or more than 10,000 unique targets within a single cell. In contrast to existing in situ detection methods, the methods presented herein have the advantage of virtually limitless numbers of individually detected molecules in parallel and in situ.

In embodiments, the method includes contacting a sample with a coupling oligonucleotide. In embodiments, the coupling oligonucleotide includes a primer binding sequence. In embodiments, the coupling oligonucleotide includes at least one primer binding sequence. In embodiments, the coupling oligonucleotide includes at least two primer binding sequences. In embodiments, the coupling oligonucleotide includes a primer binding sequence from a known set of primer binding sequences. In embodiments, the coupling oligonucleotide includes at least two primer binding sequences from a known set of primer binding sequences. In embodiments, the coupling oligonucleotide includes up to 50 different primer binding sequences from a known set of primer binding sequences. In embodiments, the coupling oligonucleotide includes up to 10 different primer binding sequences from a known set of primer binding sequences. In embodiments, the coupling oligonucleotide includes up to 5 different primer binding sequences from a known set of primer binding sequences. In embodiments, the coupling oligonucleotide includes two or more sequencing primer binding sequences from a known set of sequencing primer binding sequences. In embodiments, the coupling oligonucleotide includes 1, 2, 3, 4, 5, 6, 7, 8, 9, or 10 primer binding sequences from a known set of primer binding sequences. In embodiments, the coupling oligonucleotide includes two or more different primer binding sequences from a known set of primer binding sequences. In embodiments, the coupling oligonucleotide includes 1, 2, 3, 4, 5, 6, 7, 8, 9, or 10 different primer binding sequences from a known set of primer binding sequences. In embodiments, the coupling oligonucleotide includes 2 to 5 primer binding sequences from a known set of primer binding sequences. In embodiments, the coupling oligonucleotide includes 2 to 5 different primer binding sequences from a known set of primer binding sequences. In embodiments, the coupling oligonucleotide includes 2 to 5 sequencing primer binding sequences from a known set of sequencing primer binding sequences. In embodiments, the coupling oligonucleotide includes 2 to 5 different sequencing primer binding sequences from a known set of sequencing primer binding sequences. In embodiments, the coupling oligonucleotide includes at least two different primer binding sequences. In embodiments, the coupling oligonucleotide includes two different sequencing primer binding sequences.

In embodiments, the coupling oligonucleotide includes at least one primer binding sequence. In embodiments, the coupling oligonucleotide includes at least two primer binding sequences. In embodiments, the coupling oligonucleotide includes an amplification primer binding sequence. In embodiments, the coupling oligonucleotide includes a sequencing primer binding sequence. The amplification primer binding sequence refers to a nucleotide sequence that is complementary to a primer useful in initiating amplification (i.e., an amplification primer). Likewise, a sequencing primer binding sequence is a nucleotide sequence that is complementary to a primer useful in initiating sequencing (i.e., a sequencing primer). Primer binding sequences usually have a length in the range of between 3 to 36 nucleotides, also 5 to 24 nucleotides, also from 14 to 36 nucleotides. In embodiments, an amplification primer and a sequencing primer are complementary to the same primer binding sequence, or overlapping primer binding sequences. In embodiments, an amplification primer and a sequencing primer are complementary to different primer binding sequences.

In embodiments, the coupling oligonucleotide includes, from 5′ to 3′, a first sequence complementary to the first coupling sequence of the first oligonucleotide, a spacer sequence, and a second sequence complementary to the second coupling sequence of the second oligonucleotide. In embodiments, the coupling oligonucleotide includes 50 to 1000 nucleotides. In embodiments, the coupling oligonucleotide includes 50 to 500 nucleotides. In embodiments, the coupling oligonucleotide includes 100 to 500 nucleotides. In embodiments, the coupling oligonucleotide includes 200 to 800 nucleotides. In embodiments, the coupling oligonucleotide includes 250 to 600 nucleotides. In embodiments, the coupling oligonucleotide includes 300 to 800 nucleotides. In embodiments, the coupling oligonucleotide includes 250 to 500 nucleotides. In embodiments, the coupling oligonucleotide includes a barcode sequence (e.g., a sequence for identifying the coupling oligonucleotide). In embodiments, the coupling oligonucleotide includes about 50 nucleotides, about 60 nucleotides, about 70 nucleotides, about 80 nucleotides, about 90 nucleotides, about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, or about 200 nucleotides.

In embodiments, the coupling oligonucleotide hybridizes to sequences provided by two independent probes as described herein, wherein the probes are separated by 1-50 nm, 1-10 nm, 5-15 nm, 10⁻²⁰ nm, 15-25 nm, 20-30 nm, 25-35 nm, 30-40 nm, 35-45 nm, or 40-50 nm.

In embodiments, the coupling oligonucleotide includes one or more modified nucleotides. In embodiments, the coupling oligonucleotide includes locked nucleic acids (LNAs), Bis-locked nucleic acids (bisLNAs), twisted intercalating nucleic acids (TINAs), bridged nucleic acids (BNAs), 2′-O-methyl RNA: DNA chimeric nucleic acids, minor groove binder (MGB) nucleic acids, morpholino nucleic acids, C5-modified pyrimidine nucleic acids, peptide nucleic acids (PNAs), or combinations thereof. In embodiments, the coupling oligonucleotide includes one or more LNA nucleotides. In embodiments, the sequence complementary to the first hybridization sequence and/or the second sequence complementary to the second hybridization sequence of the coupling oligonucleotide includes one or more LNA nucleotides.

In embodiments, the method includes contacting a sample with a bridge oligonucleotide. In embodiments, the bridge oligonucleotide is a single-stranded oligonucleotide. In embodiments, the bridge oligonucleotide includes, from 3′ to 5′, a first sequence complementary to a first bridge sequence of the first oligonucleotide, a spacer sequence, and a second sequence complementary to a second bridge sequence of the second oligonucleotide.

In embodiments, the bridge oligonucleotide includes one or more modified nucleotides. In embodiments, the bridge oligonucleotide includes locked nucleic acids (LNAs), Bis-locked nucleic acids (bisLNAs), twisted intercalating nucleic acids (TINAs), bridged nucleic acids (BNAs), 2′-O-methyl RNA: DNA chimeric nucleic acids, minor groove binder (MGB) nucleic acids, morpholino nucleic acids, C₅-modified pyrimidine nucleic acids, peptide nucleic acids (PNAs), or combinations thereof. In embodiments, the bridge oligonucleotide includes one or more LNA nucleotides. In embodiments, the sequence complementary to the first hybridization sequence and/or the second sequence complementary to the second hybridization sequence of the bridge oligonucleotide includes one or more LNA nucleotides.

In embodiments, the bridge oligonucleotide includes 20 to 200 nucleotides. In embodiments, the bridge oligonucleotide includes 20 to 100 nucleotides. In embodiments, the bridge oligonucleotide includes 30 to 150 nucleotides. In embodiments, the bridge oligonucleotide includes 25 to 200 nucleotides. In embodiments, the bridge oligonucleotide includes 25 to 75 nucleotides. In embodiments, the bridge oligonucleotide includes 100 to 200 nucleotides. In embodiments, the bridge oligonucleotide includes about 50 nucleotides, about 60 nucleotides, about 70 nucleotides, about 80 nucleotides, about 90 nucleotides, about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, or about 200 nucleotides.

In embodiments, the 3′ end of the bridge oligonucleotide includes about 5 to about 50 nucleotides complementary to the 5′ end of the first independent polynucleotide. In embodiments, the 3′ end of the bridge oligonucleotide includes about 15 to about 40 nucleotides complementary to the 5′ end of the first independent polynucleotide. In embodiments, the 3′ end of the bridge oligonucleotide includes about 5, 10, 15, 20, 25, 30, 35, 40, 45, 50, or more nucleotides complementary to the 5′ end of the first independent polynucleotide.

In embodiments, the bridge oligonucleotide is about 20 to about 250 nucleotides in length. In embodiments, the bridge oligonucleotide is about 20 to 200 nucleotides, 30 to 175 nucleotides, 40 to 150 nucleotides, 50 to 125 nucleotides, or 75 to 100 nucleotides in length. In embodiments, the bridge oligonucleotide is about 20, 30, 40, 50, 75, 100, 125, 150, 175, 200, 225, 250, or more nucleotides in length.

In embodiments, the bridge oligonucleotide has an extendable 3′ end. In embodiments, the bridge oligonucleotide is extended by one or more polymerases. In embodiments, the bridge oligonucleotide is extended by a DNA polymerase, or mutant thereof. In embodiments, the bridge oligonucleotide is extended by an RNA polymerase, or mutant thereof. In embodiments, the bridge oligonucleotide is extended by a reverse transcriptase, or mutant thereof. The bridge oligo may include two hybridization sequences including or consisting of targeted priming regions flanking a linking region.

In embodiments, the method includes ligating the complement of the second barcode sequence to the first sequence of the bridge oligonucleotide (e.g., joining the 5′ and 3′ ends of each respective sequence) and ligating the complement of the first barcode sequence to form the circular oligonucleotide (e.g., joining the 5′ and 3′ ends of each respective sequence). In embodiments, the ligation includes enzymatic ligation. In embodiments, the two ends of the extended oligonucleotide primer are ligated directly together. In embodiments, the two ends of the extended oligonucleotide primer are ligated together with the aid of a bridging oligonucleotide (sometimes referred to as a splint oligonucleotide) that is complementary with the two ends of the extended oligonucleotide primer. In embodiments, ligating includes enzymatic ligation including a ligation enzyme (e.g., Circligase enzyme, Taq DNA Ligase, HiFi Taq DNA Ligase, T4 ligase, PBCV-1 DNA Ligase (also known as SplintR™ ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, ligating includes chemical ligation (e.g., enzyme-free, click-mediated ligation). In embodiments, the oligonucleotide primer includes a first bioconjugate reactive moiety capable of bonding upon contact with a second (complementary) bioconjugate reactive moiety.

In embodiments, the circular oligonucleotide is about 100 to about 1000 nucleotides in length, about 100 to about 300 nucleotides in length, about 300 to about 500 nucleotides in length, or about 500 to about 1000 nucleotides in length. In embodiments, the circular oligonucleotide is about 300 to about 600 nucleotides in length. In embodiments, the circular oligonucleotide is about 100-1000 nucleotides, about 150-950 nucleotides, about 200-900 nucleotides, about 250-850 nucleotides, about 300-800 nucleotides, about 350-750 nucleotides, about 400-700 nucleotides, or about 450-650 nucleotides in length. In embodiments, the circular oligonucleotide molecule is about 100-1000 nucleotides in length. In embodiments, the circular oligonucleotide molecule is about 100-300 nucleotides in length. In embodiments, the circular oligonucleotide molecule is about 300-500 nucleotides in length. In embodiments, the circular oligonucleotide molecule is about 500-1000 nucleotides in length. In embodiments, the circular oligonucleotide molecule is about 100 nucleotides. In embodiments, the circular oligonucleotide molecule is about 300 nucleotides. In embodiments, the circular oligonucleotide molecule is about 500 nucleotides. In embodiments, the circular oligonucleotide molecule is about 1000 nucleotides. Circular oligonucleotides may be conveniently isolated by a conventional purification column, digestion of non-circular DNA by one or more appropriate exonucleases, or both.

In embodiments, the method includes amplifying the circular oligonucleotide. In embodiments, the method includes amplifying the circular oligonucleotide in the cell or tissue. In embodiments, the method further includes amplifying the circular oligonucleotide by extending an amplification primer hybridized to the circular oligonucleotide with a strand-displacing polymerase, wherein the amplification primer extension generates an extension product including multiple complements of the circular oligonucleotide. In embodiments, the method further includes sequencing the extension product.

In embodiments, the method includes removing the circular oligonucleotide (e.g., for further processing, such as enrichment and/or amplification, and detection). In embodiments, removing the oligonucleotide includes incubation in a denaturant, for example, wherein the denaturant is a buffered solution including about 0% to about 50% dimethyl sulfoxide (DMSO); about 0% to about 50% ethylene glycol; about 0% to about 20% formamide; or about 0 to about 3M betaine, or a mixture thereof. Incubation in a denaturant should only remove the circular oligonucleotide and not remove the bound probes from the protein(s). Optimization of denaturant conditions may be performed to identify conditions suitable for selective denaturation. In embodiments, the reaction conditions are modified to denaturing conditions by i) increasing the temperature, ii) contacting the oligonucleotide with a chemical denaturant, or iii) a combination thereof.

In embodiments, amplifying includes hybridizing an amplification primer to the circular oligonucleotide and extending the primer with a strand-displacing polymerase to generate an amplification product including multiple complements of the circular oligonucleotide. In embodiments, the method includes amplifying the circular oligonucleotide by extending an amplification primer hybridized to the circular oligonucleotide with a strand-displacing polymerase, wherein the amplification primer extension generates an extension product including multiple complements of the circular oligonucleotide, including multiple complements of the first and second barcodes. In embodiments, the amplification primer extension generates an extension product including one or more complements of the circular oligonucleotide, including one or more complements of the first and second barcodes. In embodiments, the amplification primer extension generates an extension product including 2, 3, 4, 5, 6, 7, 8, 9, 10, 11, 12, 13, 14, 15, 16, 17, 18, 19, 20, or more complements of the circular oligonucleotide, including 2, 3, 4, 5, 6, 7, 8, 9, 10, 11, 12, 13, 14, 15, 16, 17, 18, 19, 20 or more complements of the first barcode and 2, 3, 4, 5, 6, 7, 8, 9, 10, 11, 12, 13, 14, 15, 16, 17, 18, 19, 20 or more complements of the second barcode. In embodiments, the method further includes sequencing the extension product.

In embodiments, the method includes amplifying the circular polynucleotide of the cell in situ. In embodiments, amplifying the circular polynucleotide generates an amplification product. In embodiments, the amplification product includes three or more copies of the circular polynucleotide. In embodiments, the amplification product includes at least three or more copies of the circular polynucleotide. In embodiments, the amplification product includes at least five or more copies of the circular polynucleotide. In embodiments, the amplification product includes at 5 to 10 copies of the circular polynucleotide. In embodiments, the amplification product includes 10 to 20 copies of the circular polynucleotide. In embodiments, the amplification product includes 20 to 50 copies of the circular polynucleotide.

In embodiments, amplifying the circular polynucleotide includes incubating the circular polynucleotide with the strand-displacing polymerase (a) for about 1 minute to about 2 hours, and/or (b) at a temperature of about 20° C. to about 50° C. In embodiments, amplifying the circular polynucleotide includes incubating the circular polynucleotide with the strand-displacing polymerase for about 1 minute to about 2 hours. In embodiments, amplifying the circular polynucleotide includes incubating the circular polynucleotide with the strand-displacing polymerase for about 5, about 10, about 20, about 30, about 40, about 45, about 50, about 55, or about 60 minutes. In embodiments, amplifying the circular polynucleotide includes incubating the circular polynucleotide with the strand-displacing polymerase for about 5 minutes. In embodiments, amplifying the circular polynucleotide includes incubating the circular polynucleotide with the strand-displacing polymerase for about 10 minutes. In embodiments, amplifying the circular polynucleotide includes incubating the circular polynucleotide with the strand-displacing polymerase for about 20 minutes. In embodiments, amplifying the circular polynucleotide includes incubating the circular polynucleotide with the strand-displacing polymerase for about 30 minutes. In embodiments, amplifying the circular polynucleotide includes incubating the circular polynucleotide with the strand-displacing polymerase for about 45 minutes. In embodiments, amplifying the circular polynucleotide includes incubating the circular polynucleotide with the strand-displacing polymerase for about 60 minutes.

In embodiments, amplifying the circular polynucleotide includes incubating the circular polynucleotide with the strand-displacing polymerase for about 1 hour to about 12 hours. In embodiments, amplifying includes incubation with the strand-displacing polymerase for about 60 seconds to about 60 minutes. In embodiments, amplifying includes incubation with the strand-displacing polymerase for about 10 minutes to about 60 minutes. In embodiments, amplifying includes incubation with the strand-displacing polymerase for about 10 minutes to about 30 minutes. In embodiments, amplifying the circular polynucleotide includes incubating the circular polynucleotide with the strand-displacing polymerase for about 1, about 2, about 3, about 4, about 5, about 6, about 7, about 8, about 9, about 10, about 11, or about 12 hours. In embodiments, amplifying the circular polynucleotide includes incubating the circular polynucleotide with the strand-displacing polymerase for more than 12 hours.

In embodiments, amplifying the circular polynucleotide includes incubating the circular polynucleotide with the strand-displacing polymerase at a temperature of about 20° C. to about 50° C. In embodiments, incubation with the strand-displacing polymerase is at a temperature of about 20° C., about 25° C., about 30° C., about 35° C., about 40° C., about 45° C., or about 50° C. In embodiments, incubation with the strand-displacing polymerase is at a temperature of about 35° C. to 42° C. In embodiments, incubation with the strand-displacing polymerase is at a temperature of about 35° C., about 36° C., about 37° C., about 38° C., about 39° C., about 40° C., about 41° C., or about 42° C. In embodiments, the strand-displacing polymerase is a phi29 polymerase, a SD polymerase, a Bst large fragment polymerase, phi29 mutant polymerase, a Thermus aquaticus polymerase, or a thermostable phi29 mutant polymerase.

In embodiments, the amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101 (43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyperbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is Bst DNA Polymerase Large Fragment, Thermus aquaticus (Taq) polymerase, or a mutant thereof.

In embodiments, the amplification method includes a standard dNTP mixture including dATP, dCTP, dGTP and dTTP (for DNA) or dATP, dCTP, dGTP and dUTP (for RNA). In embodiments, the amplification method includes a mixture of standard dNTPs and modified nucleotides that contain functional moieties (e.g., bioconjugate reactive groups) that serve as attachment points to the cell or the matrix in which the cell is embedded (e.g., a hydrogel). In embodiments, the amplification method includes a mixture of standard dNTPs and modified nucleotides that contain functional moieties (e.g., bioconjugate reactive groups) that participate in the formation of a bioconjugate linker. The modified nucleotides may react and link the amplification product to the surrounding cell scaffold. For example, amplifying may include an extension reaction wherein the polymerase incorporates a modified nucleotide into the amplification product, wherein the modified nucleotide includes a bioconjugate reactive moiety (e.g., an alkynyl moiety) attached to the nucleobase. The bioconjugate reactive moiety of the modified nucleotide participates in the formation of a bioconjugate linker by reacting with a complementary bioconjugate reactive moiety present in the cell (e.g., a crosslinking agent, such as NHS-PEG-azide, or an amine moiety) thereby attaching the amplification product to the internal scaffold of the cell. In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), malcimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, amplification reactions include standard dNTPs and a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, during amplification a mixture of standard dNTPs and aminoallyl deoxyuridine 5′-triphosphate (dUTP) nucleotides may be incorporated into the amplicon and subsequently cross-linked to the cell protein matrix by using a cross-linking reagent (e.g., an amine-reactive crosslinking agent with PEG spacers, such as (PEGylated bis(sulfosuccinimidyl) suberate) (BS(PEG) 9)).

In embodiments, amplifying includes hybridizing an amplification primer to the circular oligonucleotide and extending the primer with a non-strand-displacing polymerase to generate an amplification product. The amplification product may be isolated and processed further (e.g., capturing, enriching, and/or detecting the amplification product). It will be appreciated that any of the amplification methodologies described herein or known in the art can be utilized with universal or target-specific primers to amplify the target polynucleotide ex situ (e.g., the one or more extended polynucleotides, or circularized probes, including two or more barcodes are removed from the sample, for example the cell or tissue, and amplified on a different solid support or in solution). Suitable methods for amplification include, but are not limited to, the polymerase chain reaction (PCR), strand displacement amplification (SDA), transcription mediated amplification (TMA) and nucleic acid sequence-based amplification (NASBA), for example, as described in U.S. Pat. No. 8,003,354, which is incorporated herein by reference in its entirety. The above amplification methods can be employed to amplify one or more nucleic acids of interest ex situ. In embodiments, amplification includes an isothermal amplification reaction. In embodiments, amplification includes bridge amplification. In general, bridge amplification uses repeated steps of annealing of primers to templates, primer extension, and separation of extended primers from templates. Because primers are attached within the core polymer, the extension products released upon separation from an initial template is also attached within the core. The 3′ end of an amplification product is then permitted to anneal to a nearby reverse primer that is also attached within the core, forming a “bridge” structure. The reverse primer is then extended to produce a further template molecule that can form another bridge. In embodiments, forward and reverse primers hybridize to primer binding sites that are specific to a particular target nucleic acid. In embodiments, forward and reverse primers hybridize to primer binding sites that have been added to, and are common among, target polynucleotides. Adding a primer binding site to target nucleic acids can be accomplished by any suitable method, examples of which include the use of random primers having common 5′ sequences and ligating adapter nucleotides that include the primer binding site.

In certain embodiments the term “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In embodiments, amplifying generates an amplicon. In embodiments, an amplicon contains multiple, tandem copies of the circularized nucleic acid molecule of the corresponding sample nucleic acid. The number of copies can be varied by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. Generally, the number of copies of a nucleic acid in an amplicon is at least 100, 200, 500, 1000, 2000, 3000, 4000, 5000, 6000, 7000, 8000, 9000 and 10,000 copies, and can be varied depending on the application. As disclosed herein, one form of an amplicon is as a nucleic acid “ball” or “cluster” localized to the particle and/or well of the array. The number of copies of the nucleic acid can therefore provide a desired size of a nucleic acid “ball” or a sufficient number of copies for subsequent analysis of the amplicon, e.g., sequencing.

In embodiments of the methods provided herein, the amplicon clusters have a mean or median separation from one another of about 0.5-5 μm. In embodiments, the mean or median separation is about 0.1-10 microns, 0.25-5 microns, 0.5-2 microns, 1 micron, or a number or a range between any two of these values. In embodiments, the mean or median separation is about or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4., 4.5, 4.6, 4.7, 4.8, 4.9, 5.0 μm or a number or a range between any two of these values. The mean or median separation may be measured center-to-center (i.e., the center of one amplicon cluster to the center of a second amplicon cluster). In embodiments of the methods provided herein, the amplicon clusters have a mean or median separation (measured center-to-center) from one another of about 0.5-5 μm. The mean or median separation may be measured edge-to-edge (i.e., the edge of one amplicon cluster to the edge of a second amplicon cluster). In embodiments of the methods provided herein, the amplicon clusters have a mean or median separation (measured edge-to-edge) from one another of about 0.2-5 μm

In embodiments, the method includes detecting the amplification product. In embodiments, the method includes sequencing the amplification product. In embodiments, the method includes sequencing the circular oligonucleotide. In embodiments, the method includes sequencing the one or more barcodes, or complements thereof, of the circular oligonucleotide. In embodiments, the method includes sequencing the two or more barcodes, or complements thereof, of the circular oligonucleotide. In embodiments, the method includes sequencing the three or more barcodes, or complements thereof, of the circular oligonucleotide. Sequencing may be performed in situ or in embodiments, the circular oligonucleotide is isolated and sequenced on a separate instrument.

In embodiments, the method includes detecting the circular oligonucleotide including the first barcode sequence or complement thereof and second barcode sequence or complement thereof to detect protein interactions. In embodiments, the method includes detecting the first barcode sequence and the second barcode sequence. In embodiments, detecting includes Fluorescence In Situ Hybridization (FISH). FISH is a widely used technique for detecting short oligo sequences in situ. In FISH, oligonucleotide probes that are complementary to a sequence are labeled with a fluorescent tag. The labeled probes hybridize to the target sequence within the cells or tissue sections (e.g., in situ) allowing for the visualization and detection of the specific oligo sequence. FISH can be performed using various types of fluorophores, enabling multiplex detection of multiple oligo sequences simultaneously. In embodiments, detecting includes sequencing, for example in situ sequencing. In situ sequencing is a powerful technique that allows for the detection and characterization of short oligo sequences directly within intact cells or tissue sections. This technique combines the principles of nucleic acid hybridization, nucleotide incorporation, and imaging. In situ sequencing involves the use of sequencing-by-synthesis chemistry to read out the sequence of the target sequences directly within the sample. In embodiments, sequencing includes extending a sequencing primer by incorporating a labeled nucleotide, or labeled nucleotide analogue and detecting the label to generate a signal for each incorporated nucleotide or nucleotide analogue.

In embodiments, the method further includes sequencing each barcode to obtain a multiplexed signal in the cell in situ; demultiplexing the multiplexed signal by comparison with the known set of barcodes; and detecting the plurality of targets (e.g., the plurality of target biomolecules) by identifying the associated barcodes detected in the cell. In embodiments, demultiplexing the multiplexed signal includes a linear decomposition of the multiplexed signal. Any of a variety of techniques may be employed for decomposition of the multiplexed signal. Examples include, but are not limited to, Zimmerman et al. Chapter 5: Clearing Up the Signal: Spectral Imaging and Linear Unmixing in Fluorescence Microscopy; Confocal Microscopy: Methods and Protocols, Methods in Molecular Biology, vol. 1075 (2014); Shirawaka H. et al.; Biophysical Journal Volume 86, Issue 3, March 2004, Pages 1739-1752; and S. Schlachter, et al., Opt. Express 17, 22747-22760 (2009); the content of each of which is incorporated herein by reference in its entirety. In embodiments, multiplexed signal includes overlap of a first signal and a second signal and is computationally resolved, for example, by imaging software. In embodiments, more than one analyte type (e.g., nucleic acids and proteins) from a biological sample can be detected (e.g., simultaneously or sequentially) using any appropriate multiplexing technique.

In embodiments, sequencing includes extending a first sequencing primer to generate a sequencing read comprising the first barcode sequence, or a portion thereof. In embodiments, sequencing includes extending a first sequencing primer to generate a sequencing read comprising the first barcode sequence, or a portion thereof, and extending a second sequencing primer to generate a sequencing read comprising the second barcode sequence. In embodiments, sequencing includes sequentially extending a plurality of sequencing primers (e.g., sequencing a first barcode followed by sequencing a second barcode, followed by sequencing N barcodes, where N is the number of sequencing primers in the known sequencing primer set). In embodiments, sequencing includes generating a plurality of sequencing reads. In embodiments, following sequencing of the first barcode to a sufficient read length (e.g., the entirety of the barcode), the sequencing cycle is terminated. A sequencing cycle may be terminated by incorporated didcoxynucleoside triphosphates (ddNTPs) such that no additional nucleotides may be incorporated into an extension strand complementary to the first barcode. This process is repeated for the second, third barcodes, up to N number of primers in the set of primers. When combined with iterative sequencing (alternatively referred to as sequential sequencing) this further reduces the effects of overlapping clusters and increase detection efficiency. For example, a first primer binding site (Primer binding site 1) and a second primer binding site (Primer binding site 2) form a known primer set (i.e., a primer set of 2), and are the complementary regions for a first sequencing primer and a second sequencing primer, respectively. If six targets are present in an optically resolved volume, only half will be detected when initiating sequencing from a primer binding site, thereby increasing detection efficiency, while still allowing for overlapping target detection. If this is scaled up to a known primer set of 8 (i.e., 8 different sequencing primer binding sites and 8 corresponding sequencing primers), the individual Poisson probability of detecting one target (e.g., one RNA transcript) in a voxel increases to 42%.

In embodiments, detecting includes hybridizing a fluorescently labeled oligonucleotide to the extension product and detecting an emission light from the fluorescently labeled oligonucleotide. In embodiments, the method includes serially binding, detecting, and removing the fluorescently labeled oligonucleotide to detect a barcode sequence associated with the biomolecular interaction. For example, fluorescently labeled oligonucleotide can be removed completely after imaging by dislodging the oligonucleotides from the amplification product. In embodiments, the fluorescently labeled oligonucleotide can contain fluorophores which can be cleaved off enzymatically or chemically after imaging/detecting. The phrase “labeled probes” refers to mixture of nucleic acids that are detectably labeled, e.g., fluorescently labeled, such that the presence of the probe, as well as, any target sequence to which the probe is bound can be detected by assessing the presence of the label. In some embodiments, the probes are about 30-300 bases in length, 40-300 bases in length, or 70-300 bases in length. In some embodiments, the probes are relatively uniform in length (e.g., an average length+/−10 bases). The probes may be uniformly labeled based on position of label and/or number of labels within the probe. In some embodiments, the probes are single-stranded. In some embodiments, the probes are double-stranded. Additional detection probes and related properties may be found in, e.g., U.S. Pat. Pub. US 2011/0039735, which is incorporated herein by reference in its entirety. In embodiments, the method includes hybridizing a primer to the amplification product and incorporating a labeled nucleotide into the primer.

In embodiments, detecting includes sequencing. In embodiments, sequencing includes extending a first sequencing primer to generate a sequencing read comprising the first barcode sequence, or a portion thereof. In embodiments, sequencing includes extending a first sequencing primer to generate a sequencing read comprising the first barcode sequence, or a portion thereof, and extending a second sequencing primer to generate a sequencing read comprising the second barcode sequence. In embodiments, sequencing includes sequentially extending a plurality of sequencing primers (e.g., sequencing a first region of a target nucleic acid followed by sequencing a second region of a target nucleic acid, followed by sequencing N regions, where N is the number of sequencing primers in the known sequencing primer set). In embodiments, sequencing includes generating a plurality of sequencing reads. In embodiments, sequencing includes sequentially sequencing a plurality of different targets by initiating sequencing with different sequencing primers. For example, a first circularizable probe includes a first primer binding site (a nucleic acid sequence complementary to a first sequencing primer) and optionally a first barcode sequence or barcode nucleotide. In a similar manner, a second and third padlock probe include a second primer binding site (a nucleic acid sequence complementary to a second, different, sequencing primer) and a third primer binding site (a nucleic acid sequence complementary to a third, different from both Primer 1 and Primer 2, sequencing primer), respectively. During the first round of sequencing (following probe circularization and amplification according to the methods described herein), using primer 1, the probe hybridized to the first nucleic acid molecule is detected. In the second round of sequencing, primer 2 can hybridize and sequence an identifying sequence of the probe (e.g., a barcode sequence or nucleotide) hybridized to a second nucleic acid molecule. Similarly, in the third round of sequencing, primer 3 can hybridize and sequence the probe hybridized to the third nucleic acid molecule.

In embodiments, the method further includes sequencing the amplification product(s). Sequencing includes, for example, detecting a sequence of signals within the sample (e.g., within the cell or within the tissue). Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the readout is accomplished by epifluorescence imaging. A variety of sequencing chemistries are available, non-limiting examples of which are described herein.

In embodiments, sequencing includes extending a sequencing primer to incorporate a nucleotide containing a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product of a target nucleic acid). In embodiments, the sequencing includes sequencing-by-synthesis, sequencing-by-binding, sequencing by ligation, sequencing-by-hybridization, or pyrosequencing, and generates a sequencing read. In embodiments, generating a sequencing read includes executing a plurality of sequencing cycles, each cycle including extending the sequencing primer by incorporating a nucleotide or nucleotide analogue using a polymerase and detecting a characteristic signature indicating that the nucleotide or nucleotide analogue has been incorporated.

In embodiments, the sequencing includes extending a sequencing primer by incorporating a labeled nucleotide or labeled nucleotide analogue, and detecting the label to generate a signal for each incorporated nucleotide or nucleotide analogue, wherein the sequencing primer is hybridized to the extension product.

In embodiments, the sequencing primer includes a reversible 3′ blocking moiety. In embodiments, the reversible blocking moiety includes a dideoxy nucleoside triphosphate. In embodiments, prior to hybridizing the sequencing primer to the extension product, the reversible blocking moiety is removed, thereby generating an extendible sequencing primer. In embodiments, the sequencing primer is immobilized to a matrix or a cellular component of the cell.

In embodiments, sequencing includes a plurality of sequencing cycles. In embodiments, sequencing includes 10 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue. In embodiments, detecting includes two-dimensional (2D) or three-dimensional (3D) fluorescent microscopy. Suitable imaging technologies are known in the art, as exemplified by Larsson et al., Nat. Methods (2010) 7:395-397 and associated supplemental materials, the entire content of which is incorporated by reference herein in its entirety. In embodiments of the methods provided herein, the imaging is accomplished by confocal microscopy. Confocal fluorescence microscopy involves scanning a focused laser beam across the sample, and imaging the emission from the focal point through an appropriately-sized pinhole. This suppresses the unwanted fluorescence from sections at other depths in the sample. In embodiments, the imaging is accomplished by multi-photon microscopy (e.g., two-photon excited fluorescence or two-photon-pumped microscopy). Unlike conventional single-photon emission, multi-photon microscopy can utilize much longer excitation wavelength up to the red or near-infrared spectral region. This lower energy excitation requirement enables the implementation of semiconductor diode lasers as pump sources to significantly enhance the photostability of materials. Scanning a single focal point across the field of view is likely to be too slow for many sequencing applications. To speed up the image acquisition, an array of multiple focal points can be used. The emission from each of these focal points can be imaged onto a detector, and the time information from the scanning mirrors can be translated into image coordinates. Alternatively, the multiple focal points can be used just for the purpose of confining the fluorescence to a narrow axial section, and the emission can be imaged onto an imaging detector, such as a CCD, EMCCD, or s-CMOS detector. A scientific grade CMOS detector offers an optimal combination of sensitivity, readout speed, and low cost. One configuration used for confocal microscopy is spinning disk confocal microscopy. In 2-photon microscopy, the technique of using multiple focal points simultaneously to parallelize the readout has been called Multifocal Two-Photon Microscopy (MTPM). Several techniques for MTPM are available, with applications typically involving imaging in biological tissue. In embodiments of the methods provided herein, the imaging is accomplished by light sheet fluorescence microscopy (LSFM). In embodiments, detecting includes 3D structured illumination (3DSIM). In 3DSIM, patterned light is used for excitation, and fringes in the Moiré pattern generated by interference of the illumination pattern and the sample, are used to reconstruct the source of light in three dimensions. In order to illuminate the entire field, multiple spatial patterns are used to excite the same physical area, which are then digitally processed to reconstruct the final image. Scc York, Andrew G., et al. “Instant super-resolution imaging in live cells and embryos via analog image processing.” Nature methods 10.11 (2013): 1122-1126, which is incorporated herein by reference. In embodiments, detecting includes selective planar illumination microscopy, light sheet microscopy, emission manipulation, pinhole confocal microscopy, aperture correlation confocal microscopy, volumetric reconstruction from slices, deconvolution microscopy, or aberration-corrected multifocus microscopy. In embodiments, detecting includes digital holographic microscopy (see for example Manoharan, V. N. Frontiers of Engineering: Reports on Leading-edge Engineering from the 2009 Symposium, 2010, 5-12, which is incorporated herein by reference). In embodiments, detecting includes confocal microscopy, light sheet microscopy, or multi-photon microscopy.

In embodiments, detecting includes contacting the target of interest (e.g., a nucleic acid, protein, or biomolecule) with a fluorescently labeled probe and detecting the probe following hybridization. In embodiments, detecting includes contacting the circularized product with a fluorescently labeled probe and detecting the probe following hybridization. In embodiments, detecting includes contacting the circularized product (e.g., the circular oligonucleotide described herein) including the first barcode sequence and second barcode sequence or complement thereof with a fluorescently labeled probe and detecting the probe following hybridization. In embodiments, detecting includes contacting the amplification product with a fluorescently labeled probe and detecting the probe following hybridization. In embodiments, detecting includes contacting the sample (e.g., the sample including the circularized product and/or amplification product) with an detection solution (e.g., a buffered solution including a detectable agent, such as a fluorescently labeled probe) for about 5 minutes to about 1 hour, about 5 minutes to about 50 minutes, about 5 minutes to about 40 minutes, about 5 minutes to about 30 minutes, about 5 minutes to about 20 minutes, about 5 minutes to about 10 minutes, about 10 minutes to about 1 hour, about 10 minutes to about 50 minutes, about 10 minutes to about 40 minutes, about 10 minutes to about 30 minutes, about 10 minutes to about 20 minutes, about 20 minutes to about 1 hour, about 20 minutes to about 50 minutes, about 20 minutes to about 40 minutes, about 20 minutes to about 30 minutes, about 30 minutes to about 1 hour, about 30 minutes to about 50 minutes, about 30 minutes to about 40 minutes, about 40 minutes to about 1 hour, about 40 minutes to about 50 minutes, or about 50 minutes to about 1 hour, at a temperature of about 4° C. to about 35° C., about 4° C. to about 30° C., about 4° C. to about 25° C., about 4° C. to about 20° C., about 4° C. to about 15° C., about 4° C. to about 10° C., about 10° C. to about 35° C., about 10° C. to about 30° C., about 10° C. to about 25° C., about 10° C. to about 20° C., about 10° C. to about 15° C., about 15° C. to about 35° C., about 15° C. to about 30° C., about 15° C. to about 25° C., about 15° C. to about 20° C., about 20° C. to about 35° C., about 20° C. to about 30° C., about 20° C. to about 25° C., about 25° C. to about 35° C., about 25° C. to about 30° C., or about 30° C. to about 35° C., and detecting the detectable agent of the detection solution. The phrase “labeled probes” refers to mixture of nucleic acids that are detectably labeled, e.g., fluorescently labeled, such that the presence of the probe, as well as any target sequence to which the probe is bound, can be detected by assessing the presence of the label. In some embodiments, the probes are about 30-300 bases in length, 40-300 bases in length, or 70-300 bases in length. In some embodiments, the probes are relatively uniform in length (e.g., an average length+/−10 bases). The probes may be uniformly labeled based on position of label and/or number of labels within the probe. In some embodiments, the probes are single-stranded. In some embodiments, the probes are double-stranded. Additional detection probes and related properties may be found in, e.g., U.S. Pat. Pub. US 2011/0039735, which is incorporated herein by reference in its entirety.

In embodiments, the method includes sequencing the first and/or the second strand of an amplification product by extending a sequencing primer hybridized thereto. A variety of sequencing methodologies can be used such as sequencing-by-synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242 (1), 84-9 (1996); Ronaghi, Genome Rcs. 11 (1), 3-11 (2001); Ronaghi et al. Science 281 (5375), 363 (1998); U.S. Pat. Nos. 6,210,891; 6,258,568; and. 6,274,320, each of which are incorporated herein by reference in their entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids and amplicons thereof that are present at features of an array are subjected to repeated cycles of oligonucleotide delivery and detection. SBL methods include those described in Shendure et al. Science 309:1728-1732 (2005); U.S. Pat. Nos. 5,599,675; and 5,750,341, each of which are incorporated herein by reference in their entirety; and the SBH methodologies are as described in Bains et al., Journal of Theoretical Biology 135 (3), 303-7 (1988); Drmanac et al., Nature Biotechnology 16, 54-58 (1998); Fodor et al., Science 251 (4995), 767-773 (1995); and WO 1989/10977, each of which are incorporated herein by reference in their entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by an SBS process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072. Once such a modified nucleotide has been incorporated into the growing polynucleotide chain complementary to the region of the template being sequenced, there is no free 3′—OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934(4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, sequencing is performed according to a “sequencing-by-binding” method (scc, e.g., U.S. Pat. Pubs. US2017/0022553 and US2019/0048404, each of which is incorporated herein by reference in its entirety), which refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, car, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, car, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation.

In embodiments, the method further includes imaging the cell (e.g., obtaining bright field images (i.e., transmitted light) or dark field images (i.e., scattered light). In embodiments, the method further includes identifying and/or quantifying additional targets of interest (e.g., proteins, nucleic acids, glycolipids, or cellular structures (e.g., nucleus, mitochondria, or organelles). In embodiments, the light transmittance of the sample is measured. For example, light transmittance may be measured with a visible near-infrared optical fiber spectrometer, wherein a circular spot of light (e.g., diameter, 5 mm) is irradiated on the central part a sample and the transmitted light is collected using an optical sensor. In embodiments, the method includes obtaining cell images for analysis of cell morphology. In embodiments, a plurality of cells are immobilized in a 96-well microplate having a mean or median well-to-well spacing of about 8 mm to about 12 mm (e.g., about 9 mm). In embodiments, a plurality of cells is immobilized in a 384-well microplate having a mean or median well-to-well spacing of about 3 mm to about 6 mm (e.g., about 4.5 mm). In embodiments, the device as described herein detects scattered light from the sample. In embodiments, the device as described herein detects diffracted light from the sample. In embodiments, the device as described herein detects reflected light from the sample. In embodiments, the device as described herein detects absorbed light from the sample. In embodiments, the device as described herein detects refracted light from the sample. In embodiments, the device as described herein detects transmitted light not absorbed by the sample. In embodiments, the sample does not include a label. In embodiments, the methods and system as described herein detect scattered light from the sample. In embodiments, the methods and system as described herein detect diffracted light from the sample. In embodiments, the methods and system as described herein detect reflected light from the sample. In embodiments, the methods and system as described herein detect absorbed light from the sample. In embodiments, the methods and system as described herein detect refracted light from the sample. In embodiments, the methods and system as described herein detect transmitted light not absorbed by the sample. In embodiments, the device is configured to determine the cell morphology (e.g., the cell boundary, granularity, or cell shape). For example, to determining the cell boundary includes comparing the pixel values of an image to a single intensity threshold, which may be determined quickly using histogram-based approaches as described in Carpenter, A. et al., Genome Biology 7, R100 (2006) and Arce, S., Sci Rep 3, 2266 (2013).

In embodiments, the method further includes an additional imaging modality, immunofluorescence (IF), or immunohistochemistry modality (e.g., immunostaining). In embodiments, the method includes hematoxylin and eosin (H&E) staining. In embodiments, the method includes ER staining (e.g., contacting the cell with a cell-permeable dye which localizes to the endoplasmic reticula), Golgi staining (e.g., contacting the cell with a cell-permeable dye which localizes to the Golgi), F-actin staining (e.g., contacting the cell with a phalloidin-conjugated dye that binds to actin filaments), lysosomal staining (e.g., contacting the cell with a cell-permeable dye that accumulates in the lysosome via the lysosome pH gradient), mitochondrial staining (e.g., contacting the cell with a cell-permeable dye which localizes to the mitochondria), nucleolar staining, or plasma membrane staining. For example, the method includes live cell imaging (e.g., obtaining images of the cell) prior to or during fixing, immobilizing, and permeabilizing the cell. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology (e.g., the cell boundary or cell shape) using known methods in the art.

In embodiments, the cell is imaged using “optical sectioning” techniques, such as laser scanning confocal microscopes, laser scanning 2-Photon microscopy, parallelized confocal (i.e. spinning disk), computational image deconvolution methods, and light sheet approaches. Optical sectioning microscopy methods provide information about single planes of a volume by minimizing contributions from other parts of the volume and do so without physical sectioning. The resulting “stack” of such optically sectioned images, represents a full reconstruction of the 3-dimensional features of a tissue volume. A typical confocal microscope includes a 10×/0.5 objective (dry; working distance, 2.0 mm) and/or a 20×/0.8 objective (dry; working distance, 0.55 mm), with a z-step interval of 1 to 5 μm. A typical light sheet fluorescence microscope includes an sCMOS camera, a 2×/0.5 objective lens, and zoom microscope body (magnification range of ×0.63 to ×6.3). For entire scanning of whole samples, the z-step interval is 5 or 10 μm, and for image acquisition in the regions of interest, an interval in the range of 2 to 5 μm may be used.

In embodiments, the method includes performing an additional image processing techniques (e.g., filtering, masking, smoothing, UnSharp Mask filter (USM), deconvolution, or maximum intensity projection (MIP)). In embodiments, the method includes computationally filtering the emissions using a linear or nonlinear filter that amplifies the high-frequency components of the emission. For example, USM method applies a Gaussian blur to a duplicate of the original image and then compares it to the original. If the difference is greater than a threshold setting, the images are subtracted. In embodiments, the method includes a maximum intensity projection (MIP). A maximum intensity projection is a visualization technique that takes three-dimensional data (e.g., emissions from varying depths obtained according to the methods described herein) and turns it into a single two-dimensional image. For example, the projection takes the brightest pixel (voxel) in each depth and displays that pixel intensity value in the final two-dimensional image. Various machine learning approaches may be used, for example, the methods described in Lugagne et al. Sci Rep 8, 11455 (2018) and Pattaronc, G., et al. Sci Rep 11, 10304 (2021), each of which are incorporated herein by reference. In embodiments, the method includes focus stacking (e.g., z-stacking) which combines multiple images taken at different focus distances to give a resulting image with a greater depth of field (DOF) than any of the individual source images. The devices and methods described herein provide for the detection analytes and analyte levels (e.g., gene and/or protein expression) within or on different cells in a tissue of a mammal or within a single cell. For example, the methods can be used to detect analytes (e.g., genes and/or proteins) within or on different cells in histological slide samples, the data from which can be reassembled to generate a three-dimensional map of analytes of a tissue sample.

In embodiments, the method includes fixing and/or staining the sample. In embodiments of any of the methods described herein, the non-permeabilized biological sample is fixed and/or stained prior. In embodiments, the step of fixing the sample includes the use of a fixative (e.g., contacting and/or incubating with the sample) such as ethanol, methanol, acetone, formaldehyde, paraformaldehyde-Triton, glutaraldehyde, and combinations thereof. In embodiments, the staining the sample includes contacting and/or incubating with the sample acridine orange, Bismarck brown, carmine, coomassie blue, cresyl violet, DAPI, eosin, cthidium bromide, acid fuchsin, hematoxylin, Hoechst stains, iodine, methyl green, methylene blue, neutral red, Nile blue, Nile red, osmium tetroxide, propidium iodide, rhodamine, safranin, and combinations thereof. In embodiments, staining includes contacting the sample with eosin and hematoxylin. In embodiments, staining includes contacting the sample with a detectable label selected from the group consisting of a radioisotope, a fluorophore, a chemiluminescent compound, a bioluminescent compound, or a combination thereof.

In embodiments, the methods described herein may further include constructing a 3-dimensional pattern of abundance, expression, and/or activity of each target from spatial patterns of abundance, expression, and/or activity of each target of multiple samples. In embodiments, the multiple samples can be consecutive tissue sections of a 3-dimensional tissue sample.

In embodiments, the method further includes removing the embedding material from the sample. For example, if the embedding material is paraffin wax, the embedding material is removed by contacting the sample-carrier construct with a hydrocarbon solvent, such as xylene or hexane, followed by two or more washes with decreasing concentrations of an alcohol, such as ethanol.

In an aspect is provided a method of sequencing two barcode sequences. In embodiments, the method includes a) binding a first probe to a first target molecule and binding a second probe to a second target molecule, first probe attached to a first oligonucleotide including a first barcode sequence and the second probe attached to a second oligonucleotide including a second barcode sequence; b) forming a circular oligonucleotide including complements of the first barcode sequence and the second barcode sequence; c) amplifying the circular oligonucleotide to form amplification products including the first barcode sequence and the second barcode sequence (e.g., forming a unified barcode); and d) hybridizing a sequencing primer to an amplification product and sequencing the first barcode sequence and the second barcode sequence. In embodiments, a)-d) are performed in situ (e.g., in a cell, on a cell, or in a tissue). In embodiments, the method includes detecting a plurality of different targets within an optically resolved volume of a cell in situ. In embodiments, the method includes associating a different oligonucleotide barcode from a known set of barcodes with each of the plurality of targets; ii) sequencing each barcode to obtain a multiplexed signal in situ; iii) demultiplexing the multiplexed signal by comparison with the known set of barcodes; and detecting the plurality of targets by identifying the associated barcodes detected in the cell.

In embodiments, the method includes detecting multiple target molecules on the surface of a cell or tissue using a probe or plurality of probes described. In embodiments, the method includes detecting multiple target molecules within a cell or tissue using a probe or plurality of probes described. In embodiments, the method includes detecting multiple target molecules from a cell or tissue using a probe or plurality of probes described. In embodiments, the method includes detecting multiple target molecules from a cell or tissue using a probe or plurality of probes described herein includes detecting proteins or oligonucleotides. In embodiments, the method includes detecting multiple target molecules from a cell or tissue using a probe or plurality of probes described herein includes detecting proteins. In embodiments, the method includes detecting multiple target molecules from a cell or tissue using a probe or plurality of probes described herein includes detecting cell surface markers. In embodiments, the method includes detecting multiple target molecules from a cell or tissue using a probe or plurality of probes described herein includes detecting intracellular organelles. In embodiments, the method includes detecting multiple target molecules from a cell or tissue using a probe or plurality of probes described herein includes detecting oligonucleotides. In embodiments, the method further includes imaging the cell and determining a location of the first target molecule and the second target molecule.

In embodiments, the first target molecule and the second target molecule are different (e.g., different and distinct molecules). In embodiments, the first target molecule and the second target molecule are part of the same target molecule (e.g., see FIG. 2E). In embodiments, the target molecule is located in a cell or tissue. In embodiments, the target molecule is located on the surface of a cell. In embodiments, the target molecule is a protein. In embodiments, the target molecule is an oligonucleotide. In embodiments, the target molecule is a carbohydrate. In embodiments, the target molecule is a lipid. In embodiments, the target molecule is an RNA nucleic acid sequence. In embodiments, the target molecule is a DNA nucleic acid sequence. In embodiments, the target molecule is a nucleic acid. In embodiments, the target molecule is a non-nucleic acid target. Non-nucleic acid targets include, but are not limited to, lipids, carbohydrates, peptides, proteins, glycoproteins, lipoproteins, phosphoproteins, acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In embodiments, the target molecule is an organelle. In embodiments, the target molecule is located within the cell. In embodiments, the target molecule is located on the surface of the cell. In embodiments, the target molecule is attached on a cell surface, such as a transmembrane analyte. In embodiments, target molecule detected using the method described herein are each independently different. In embodiments, the first target molecule is an RNA molecule and the second target molecule is a protein. In embodiments, the first target molecule is a protein and the second target molecule is a protein.

In embodiments, the method includes computationally grouping target molecules based on the combined detection of the two barcode sequences. In embodiments, the method includes quantifying the amount of target molecules.

### III. Compositions & Kits

In an aspect is provided a circular oligonucleotide (e.g., a circular oligonucleotide including two barcode sequences) as described herein. In embodiments, the circular oligonucleotide includes the sequence of a first barcode as described herein or complement thereof, a second barcode as described herein or complement thereof, coupling oligonucleotide as described herein, and bridge oligonucleotide as described herein. In embodiments, the circular oligonucleotide is in a cell or tissue. In embodiments, the circular oligonucleotide is on a cell. In embodiments, the circular oligonucleotide is in a tissue.

In another aspect is provided a protein-probe complex, wherein the protein-probe complex includes a protein bound to a first probe and a second probe, the first probe attached to a first oligonucleotide including a first barcode sequence and the second probe attached to a second oligonucleotide including a second barcode sequence. In embodiments, the protein-probe complex is in a cell or tissue. In embodiments, the protein-probe complex is on a cell or tissue.

In an aspect is provided a linked complex including the protein-probe complex as described herein and the coupling oligonucleotide as described herein. In embodiments, the protein-probe complexes include one or more proteins. In embodiments, the protein-probe complexes include one, two, three, or four proteins. In embodiments, the protein-probe complexes include one or more probes. In embodiments, the protein-probe complexes include two, two, three, or four probes. In embodiments, the protein-probe complexes include two probes. In embodiments, the probes in the protein-probe complex binds to different proteins (see, e.g., FIG. 1D). In embodiments, the probes in the protein-probe complex binds to different regions on the same protein (scc, e.g., FIG. 1E). In embodiments, the probe is attached to an oligonucleotide comprising a barcode sequence, a sequence capable of hybridizing with a sequence in a coupling oligonucleotide described herein, and a sequence capable of hybridizing with a sequence in a bridge oligonucleotide described herein, wherein the oligonucleotide is attached to the probe at the 3′ end of the oligonucleotide (see, e.g., FIG. 2C). In embodiments, the probe is attached to an oligonucleotide comprising a barcode sequence, a sequence capable of hybridizing with a sequence in a coupling oligonucleotide described herein, and a sequence capable of hybridizing with a sequence in a bridge oligonucleotide described herein, wherein the oligonucleotide is attached to the probe at the 5′ end of the oligonucleotide (see, e.g., FIG. 1C). In embodiments, the linked complex is in a cell or tissue. In embodiments, the linked complex is on a cell or tissue.

In an aspect is provided a linked-bridge complex including the linked complex as described herein and bridge oligonucleotide as described herein. In embodiments, the linked-bridge complex is in a cell or tissue. In embodiments, the linked-bridge complex is on a cell or tissue.

In an aspect is provided a coupling oligonucleotide (e.g., a coupling oligonucleotide as described herein). In embodiments, the coupling oligonucleotide is in a cell or tissue. In embodiments, the coupling oligonucleotide is on a cell or tissue.

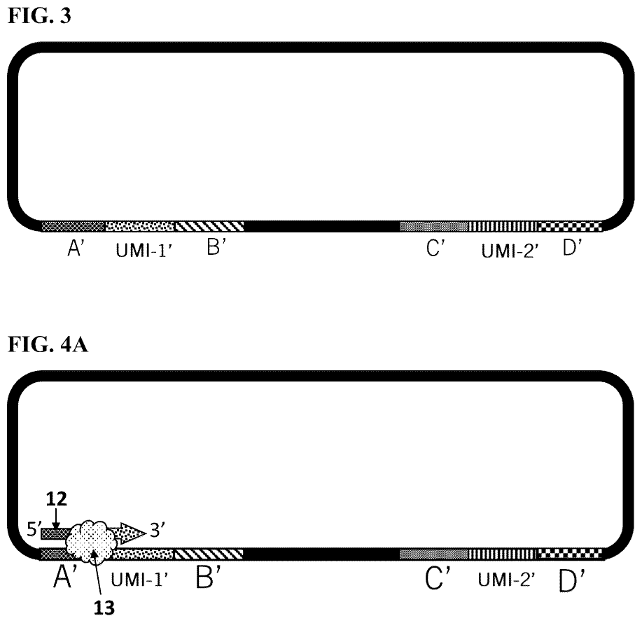

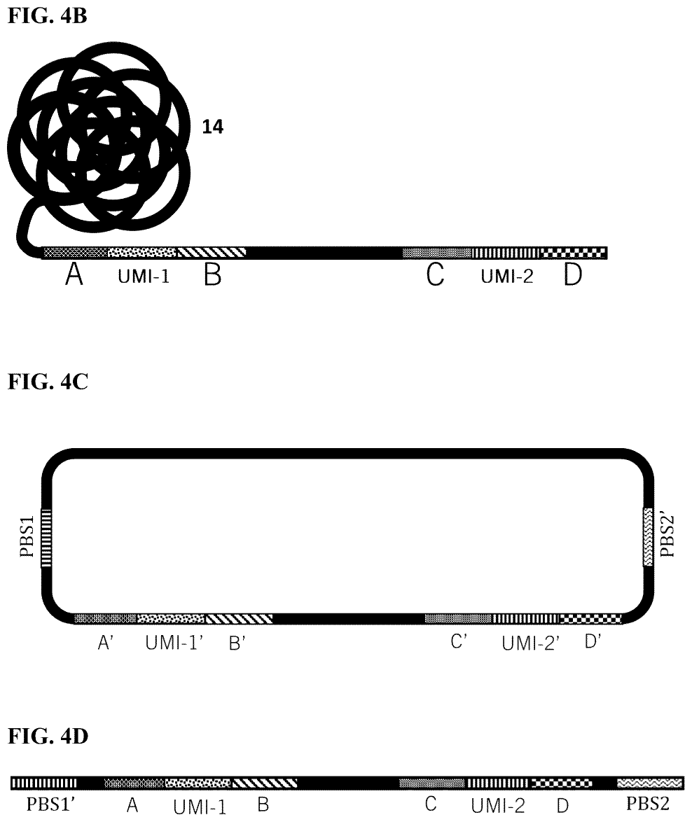

In an aspect is provided a circular oligonucleotide (e.g., a circular oligonucleotide including two barcode sequences) as described herein. In embodiments, the circular oligonucleotide may be formed when the Universal Bridge oligo anneals to two or more tag oligos which, in turn, anneal to the ends of a PLP, followed by extension and ligation. In embodiments, the circular oligonucleotide includes a primer binding sequence. In embodiments, the circular oligonucleotide includes at least one primer binding sequence. In embodiments, the circular oligonucleotide includes two primer binding sequences, such as is illustrated in FIG. 4C (PBS1 and PBS2′ in this particular, nonlimiting example). In embodiments, the circular oligonucleotide includes more than two primer binding sequences. In embodiments, the circular oligonucleotide includes a primer binding sequence from a known set of primer binding sequences. In embodiments, the circular oligonucleotide includes at least two primer binding sequences from a known set of primer binding sequences. In embodiments, the circular oligonucleotide includes up to 50 different primer binding sequences from a known set of primer binding sequences. In embodiments, the circular oligonucleotide includes up to 10 different primer binding sequences from a known set of primer binding sequences. In embodiments, the circular oligonucleotide includes up to 5 different primer binding sequences from a known set of primer binding sequences. In embodiments, the circular oligonucleotide includes two or more sequencing primer binding sequences from a known set of sequencing primer binding sequences. In embodiments, the circular oligonucleotide includes 1, 2, 3, 4, 5, 6, 7, 8, 9, or 10 primer binding sequences from a known set of primer binding sequences. In embodiments, the circular oligonucleotide includes two or more different primer binding sequences from a known set of primer binding sequences. In embodiments, the circular oligonucleotide includes 1, 2, 3, 4, 5, 6, 7, 8, 9, or 10 different primer binding sequences from a known set of primer binding sequences. In embodiments, the circular oligonucleotide includes 2 to 5 primer binding sequences from a known set of primer binding sequences. In embodiments, the circular oligonucleotide includes 2 to 5 different primer binding sequences from a known set of primer binding sequences. In embodiments, the circular oligonucleotide includes 2 to 5 sequencing primer binding sequences from a known set of sequencing primer binding sequences. In embodiments, the circular oligonucleotide includes 2 to 5 different sequencing primer binding sequences from a known set of sequencing primer binding sequences. In embodiments, the circular oligonucleotide includes at least two different primer binding sequences. In embodiments, the circular oligonucleotide includes two different sequencing primer binding sequences. In embodiments, the circular oligonucleotide includes an amplification primer binding sequence. In embodiments, the circular oligonucleotide includes a sequencing primer binding sequence. The amplification primer binding sequence refers to a nucleotide sequence that is complementary to a primer useful in initiating amplification (i.e., an amplification primer). Likewise, a sequencing primer binding sequence is a nucleotide sequence that is complementary to a primer useful in initiating sequencing (i.e., a sequencing primer). Primer binding sequences usually have a length in the range of between 3 to 36 nucleotides, also 5 to 24 nucleotides, also from 14 to 36 nucleotides. In embodiments, an amplification primer and a sequencing primer are complementary to the same primer binding sequence, or overlapping primer binding sequences. In embodiments, an amplification primer and a sequencing primer are complementary to different primer binding sequences.

In embodiments, one or more barcode sequences within the circular polynucleotide is 1 to 20 nucleotides. In embodiments, one or more barcode sequences within the circular polynucleotide is at least 5, 6, 7, 8, 9, 10, 11, 12, 13, 14, or 15 nucleotides in length. In embodiments, one or more barcode sequences within the circular polynucleotide is 5, 6, 7, 8, 9, 10, 11, 12, 13, 14, or 15 nucleotides in length. In embodiments, one or more barcode sequences within the circular polynucleotide is 10 to 15 nucleotides in length. In embodiments, one or more barcode sequences within the circular polynucleotide is at least about 1, 2, 3, 4, 5, 6, 7, 8, 9, 10, 15, 20, 25, 30, 35, 40, 45, 50 or more nucleotides in length. In embodiments, one or more barcode sequences within the circular polynucleotide can be at most about 300, 200, 100, 90, 80, 70, 60, 50, 40, 30, 20, 15, 12, 10, 9, 8, 7, 6, 5, 4 or fewer or more nucleotides in length. In embodiments, one or more barcode sequences within the circular polynucleotide includes between about 5 to about 8, about 5 to about 10, about 5 to about 15, about 5 to about 20, about 10 to about 150 nucleotides. In embodiments, one or more barcode sequences within the circular polynucleotide includes between 5 to 8, 5 to 10, 5 to 15, 5 to 20, 10 to 150 nucleotides. In embodiments, one or more barcode sequences within the circular polynucleotide is 10 nucleotides. In embodiments, one or more barcode sequences within the circular polynucleotide may include a unique sequence (e.g., a barcode sequence) that gives the barcode its identifying functionality. In embodiments, one or more barcode sequences within the circular polynucleotide may be random or non-random.

In embodiments, one or more barcode sequences within the circular polynucleotide consists only of a unique barcode sequence. In embodiments, one or more barcode sequences within the circular polynucleotide can be a nucleic acid sequence which associates the oligonucleotide barcode with the protein(s) of interest. In embodiments, one or more barcode sequences within the circular polynucleotide includes a first and second barcode sequence such as is illustrated in FIG. 3 and/or FIG. 4C. In embodiments, one or more barcode sequences within the circular polynucleotide include a unified barcode formed via the combination of two or more subsequences such as is illustrated in FIGS. 5 (15 and 16). In embodiments, one or more unified barcode sequences within the circular polynucleotide may have a length of 10 to 40, 10 to 30, 10 to 20, 25 to 50, 15 to 40, 15 to 30, 20 to 50, 20 to 40, or 20 to 30 nucleotides. In embodiments, one or more barcode sequences within the circular polynucleotide is a degenerate or partially-degenerate sequence, such that one or more nucleotides are selected at random from a set of two or more different nucleotides at one or more positions, with each of the different nucleotides selected at one or more positions represented in a pool of oligonucleotides including the degenerate or partially-degenerate sequence.

In some embodiments, one or more of the specific binding molecular moieties of the barcoded probes within the protein-probe complex is an antibody, a lectin, or an aptamer. In some embodiments, one or more of the specific binding molecular moieties of the barcoded probes within the protein-probe complex is an antibody. In some embodiments, one or more of the specific binding molecular moieties of the barcoded probes within the protein-probe complex is an aptamer. In some embodiments, one or more of the specific binding molecular moieties of the barcoded probes within the protein-probe complex is a polyclonal antibody. In some embodiments, one or more of the specific binding molecular moieties of the barcoded probes within the protein-probe complex is a monoclonal antibody. In some embodiments, one or more of the specific binding molecular moieties of the barcoded probes within the protein-probe complex is a monobody. In some embodiments, one or more of the specific binding molecular moieties of the barcoded probes within the protein-probe complex is an affibody.

In some embodiments, the barcoded probes within the protein-probe complex bind to a single target protein. In some embodiments, the barcoded probes within the protein-probe complex bind to one epitope of a single target protein. In some embodiments, the barcoded probes within the protein-probe complex bind to two epitopes of a single target protein (nonlimiting examples of which are illustrated in FIG. 1E and FIG. 2E). In some embodiments, the barcoded probes within the protein-probe complex bind to more than two epitopes of a single target protein. In some embodiments, the barcoded probes within the protein-probe complex bind to two target proteins in close proximity to each other. In some embodiments, the barcoded probes within the protein-probe complex bind to two target proteins in close proximity to each other which are directly bound to each other (nonlimiting examples of which are illustrated in FIG. 1D, FIG. 2D, and FIG. 5). In some embodiments, the barcoded probes within the protein-probe complex bind to two target proteins in close proximity to each other which are not directly bound to each other. In some embodiments, the barcoded probes within the protein-probe complex bind to two target proteins in close proximity to each other which are not directly bound to each other, but are both bound to a third protein (e.g., separated by one protein, a nonlimiting examples of which is illustrated in FIG. 6B). In some embodiments, the barcoded probes within the protein-probe complex bind to two target proteins in close proximity to each other which are not directly bound to each other, but are separated by two proteins (a nonlimiting examples of which is illustrated in FIG. 6A). In some embodiments, the barcoded probes within the protein-probe complex bind to two target proteins in close proximity to each other which are not directly bound to each other, but are separated by more than two proteins.

In some embodiments, at least some of the barcoded probes within the protein-probe complex bind to one or more target proteins. In some embodiments, at least some of the barcoded probes within the protein-probe complex bind to one or more non-protein targets. In some embodiments, at least some of the barcoded probes within the protein-probe complex bind to one or more carbohydrate targets (a nonlimiting example of which is illustrated in FIG. 6C).

In an aspect is provided a kit. In embodiments, the kit includes a composition as described herein. In embodiments, the kit includes the reagents and containers useful for performing the methods as described herein. Generally, the kit includes one or more containers providing a composition and one or more additional reagents (e.g., a buffer suitable for polynucleotide extension and/or sequencing). The kit may also include a template nucleic acid (DNA and/or RNA), one or more primer polynucleotides, nucleoside triphosphates (including, e.g., deoxyribonucleotides, ribonucleotides, labeled nucleotides, and/or modified nucleotides), buffers, salts, and/or labels (e.g., fluorophores). In embodiments, the kit includes the coupling oligonucleotide described herein, the bridge oligonucleotide described herein, a plurality of the first probe, and a plurality of the second probe. In embodiments, the kit includes a plurality of one, two, three, four, five, six, seven, eight, nine, ten, eleven, twelve, or more probes.

In embodiments, the kit includes a solid support. In embodiments, the kit includes a solid support including a cell or tissue immobilized to the surface of the solid support. In embodiments, the cell or tissue immobilized to the surface of the solid support includes protein-probe complex described herein. In embodiments, the cell or tissue immobilized to the surface of the solid support includes linked complex described herein. In embodiments, the cell or tissue immobilized to the surface of the solid support includes linked-bridge complex described herein. In embodiments, kit includes a solid support, wherein the solid support includes a functionalized glass surface or a functionalized plastic surface (e.g., a surface including a plurality of reactive moictics).

In embodiments, the kit includes a multiwell container, a microplate, and/or reagents for sample preparation and purification, amplification, and/or sequencing (e.g., one or more sequencing reaction mixtures). In embodiments, the kit includes for protein detection includes a plurality of probes linked to an oligonucleotide (e.g., DNA-conjugated antibodies).

In embodiments, amplification reagents and other reagents may be provided in lyophilized form. In embodiments, amplification reagents and other reagents may be provided in a container that includes wells within which the lyophilized reagent may be reconstituted.

In embodiments, the kit includes components useful for circularizing template polynucleotides using a ligation enzyme (e.g., Circligase enzyme, Taq DNA Ligase, HiFi Taq DNA Ligase, T4 ligase, SplintR ligase, or Ampligase DNA Ligase). For example, such a kit further includes the following components: (a) reaction buffer for controlling pH and providing an optimized salt composition for a ligation enzyme (e.g., Circligase enzyme, Taq DNA Ligase, HiFi Taq DNA Ligase, T4 ligase, SplintR ligase, or Ampligase DNA Ligasc), and (b) ligation enzyme cofactors. In embodiments, the kit further includes instructions for use thereof. In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archacal DNA polymerase. In embodiments, the kit includes a sequencing solution. In embodiments, the sequencing solution include labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof; and a guanine nucleotide, or analog thereof may be labeled with a different fluorescent label. In embodiments, the kit includes a modified terminal deoxynucleotidyl transferase (TdT) enzyme.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg2+, Mn2+, Zn2+, and Ca2+. In embodiments, the buffered solution can contain one or more divalent cations at a concentration sufficient to permit hybridization of a nucleic acid. In embodiments, the buffered solution can contain one or more divalent cations at a concentration sufficient to permit hybridization of a nucleic acid. In embodiments, the buffered solution includes about 10 mM Tris, about 20 mM Tris, about 30 mM Tris, about 40 mM Tris, or about 50 mM Tris. In embodiments the buffered solution includes about 50 mM NaCl, about 75 mM NaCl, about 100 mM NaCl, about 125 mM NaCl, about 150 mM NaCl, about 200 mM NaCl, about 300 mM NaCl, about 400 mM NaCl, or about 500 mM NaCl. In embodiments, the buffered solution includes about 0.05 mM EDTA, about 0.1 mM EDTA, about 0.25 mM EDTA, about 0.5 mM EDTA, about 1.0 mM EDTA, about 1.5 mM EDTA or about 2.0 mM EDTA. In embodiments, the buffered solution includes about 0.01% Triton X-100, about 0.025% Triton X-100, about 0.05% Triton X-100, about 0.1% Triton X-100, or about 0.5% Triton X-100. In embodiments, the buffered solution includes 20 mM Tris pH 8.0, 100 mM NaCl, 0.1 mM EDTA, 0.025% Triton X-100. In embodiments, the buffered solution includes 20 mM Tris pH 8.0, 150 mM NaCl, 0.1 mM EDTA, 0.025% Triton X-100. In embodiments, the buffered solution includes a poloxamer. In embodiments, the buffered solution includes about 0.002% Pluronic® F-127, about 0.01% Pluronic® F-127, about 0.02% Pluronic® F-127, about 0.05% Pluronic® F-127, about 0.1% Pluronic® F-127, about 0.2% Pluronic® F-127, about 0.3% Pluronic® F-127, about 0.4% Pluronic® F-127, about 0.5% Pluronic® F-127, about 0.6% Pluronic® F-127, about 0.7% Pluronic® F-127, about 0.8% Pluronic® F-127, about 0.9% Pluronic® F-127, about 1% Pluronic® F-127, about 1.1% Pluronic® F-127, about 1.2% Pluronic® F-127, about 1.3% Pluronic® F-127, about 1.4% Pluronic® F-127, about 1.5% Pluronic® F-127, about 1.6% Pluronic® F-127, about 1.7% Pluronic® F-127, about 1.8% Pluronic® F-127, about 1.9% Pluronic® F-127, or about 2% Pluronic® F-127. In embodiments, the buffered solution includes 0.1 mM DTT, 0.5 mM DTT, 1 mM DTT, 2 mM DTT, 3 mM DTT, 4 mM DTT, 5 mM DTT, 6 mM DTT, 7 mM DTT, 8 mM DTT, 9 mM DTT, 10 mM DTT, 11 mM DTT, 12 mM DTT, 13 mM DTT, 14 mM DTT, 15 mM DTT, 16 mM DTT, 17 mM DTT, 18 mM DTT, 19 mM DTT, or 20 mM DTT. Triton™ is a registered trademark of Dow Chemical Company. In embodiments, the buffered solution includes about 1 mM MgCl2, about 2 mM MgCl2, about 3 mM MgCl2, about 4 mM MgCl2, about 5 mM MgCl2, about 6 mM MgCl2, about 7 mM MgCl2, about 8 mM MgCl2, about 9 mM MgCl2, about 10 mM MgCl2, about 11 mM MgCl2, about 12 mM MgCl2, about 13 mM MgCl2, about 14 mM MgCl2, about 15 mM MgCl2, about 16 mM MgCl2, about 17 mM MgCl2, about 18 mM MgCl2, about 19 mM MgCl2, or about 20 mM MgCl2. In embodiments, the buffered solution includes about 0.01 mM ATP, about 0.05 mM ATP, about 0.1 mM ATP, about 0.25 mM ATP, about 0.5 mM ATP, about 0.75 mM ATP, about 1 mM ATP, about 2 mM ATP, about 3 mM ATP, about 4 mM ATP, about 5 mM ATP, about 6 mM ATP, about 7 mM ATP, about 8 mM ATP, about 9 mM ATP, or about 10 mM ATP. In embodiments, the buffered solution includes about 25 mM LiCI, about 50 mM LiCI, about 75 mM LiCI, about 100 mM LiCI, about 125 mM LiCI, about 150 mM LiCI, about 175 mM LiCI, about 200 mM LiCI, about 225 mM LiCI, about 250 mM LiCI, about 275 mM LiCI, about 300 mM LiCI, about 325 mM LiCI, about 350 mM LiCI, about 375 mM LiCI, about 400 mM LiCI, about 425 mM LiCI, about 450 mM LiCI, about 475 mM LiCI, or about 500 mM LiCl. In embodiments, the buffered solution includes 20 mM Tris pH 8.0, 300 mM NaCl, 0.1 mM EDTA, 0.025% Triton X-100. In embodiments, the buffered solution includes 20 mM Tris pH 8.0, 400 mM NaCl, 0.1 mM EDTA, 0.025% Triton X-100. In embodiments, the buffered solution includes 20 mM Tris pH 8.0, 500 mM NaCl, 0.1 mM EDTA, 0.025% Triton X-100.

In embodiments, the kit includes one or more sequencing reaction mixtures. In embodiments, the sequencing reaction mixture includes a buffer. In embodiments, the buffer includes an acetate buffer, 3-(N-morpholino) propanesulfonic acid (MOPS) buffer, N-(2-Acctamido)-2-aminocthanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminocthanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site. Any convenient means may be present in the kits.

In embodiments, the kit can further include one or more biological stain(s) (e.g., any of the biological stains as described herein). For example, the kit can further include eosin and hematoxylin. In other examples, the kit can include a biological stain such as acridine orange, Bismarck brown, carmine, coomassie blue, cresyl violet, DAPI, eosin, ethidium bromide, acid fuchsin, hematoxylin, Hoechst stains, iodine, methyl green, methylene blue, neutral red, Nile blue, Nile red, osmium tetroxide, propidium iodide, rhodamine, safranin, or any combination thereof.

In an aspect is provided a cell or tissue including a circular oligonucleotide (e.g., a circular oligonucleotide as described herein) in or on the cell or tissue. In embodiments, the circular oligonucleotide includes the sequence of a first barcode or complement thereof, a second barcode or complement thereof, coupling oligonucleotide, and bridge oligonucleotide. In embodiments, the cell or tissue includes a plurality of circular oligonucleotides, wherein a circular oligonucleotide in the plurality of circular oligonucleotide harbors a distinct first barcode or complement thereof and a distinct second barcode or complement thereof to permit differentiation between one circular oligonucleotide from another circular oligonucleotide in a plurality of circular oligonucleotides.

In embodiments, the tissue includes liver tissue, kidney tissue, bone tissue, lung tissue, thymus tissue, adrenal tissue, skin tissue, bladder tissue, colon tissue, spleen tissue, or brain tissue.

In embodiments, the method detects interactions between proteins and smaller molecules such as hormones, drugs, or neurotransmitters. This is accomplished by conjugating an oligonucleotide to a protein receptor and using a ligand or a small molecule conjugated to a complementary oligonucleotide. Upon ligand binding to its receptor, the proximity of the conjugates facilitates the necessary steps described herein for signal generation and detection, useful in pharmacology and biochemical signaling studies.

In embodiments, the method further includes simultaneously detecting multiple interaction types (e.g., RNA-RNA, RNA-protein, DNA-protein, protein-small molecule) in a single assay. This involves using a panel of different specific binding agent-oligonucleotide conjugates, each specific binding agent pertaining to different classes of molecules.

In an aspect is provided a microfluidic device including a solid support. In embodiments, the solid support includes a cell or tissue including: a first probe bound to a first target molecule and a second probe bound to a second target molecule, wherein the first probe is attached to a first oligonucleotide including a first barcode sequence and the second probe attached to a second oligonucleotide including a second barcode sequence; a coupling oligonucleotide hybridized to the first oligonucleotide and to the second oligonucleotide; and a bridge oligonucleotide hybridized to the first oligonucleotide and to the second oligonucleotide. In embodiments, the microfluidic device includes a flow cell. In embodiments, the microfluidic device includes an imaging system or detection apparatus. Exemplary systems having fluidic and detection components that can be readily modified for use in a system herein include, but are not limited to, those set forth in U.S. Pat. Nos. 8,241,573, 8,039,817; or US Pat. App. Pub. No. 2012/0270305 A1, each of which is incorporated herein by reference. In embodiments, the microfluidic device further includes one or more excitation lasers.

In embodiments, the microfluidic device is a nucleic acid sequencing device including: a stage configured to hold an array or solid support as described herein, including embodiments; an array or solid support as described herein, including embodiments; and a detector for obtaining sequencing data. In some embodiments, the detector is an imaging detector, such as a CCD, EMCCD, or s-CMOS detector. Nucleic acid sequencing devices utilize excitation beams to excite labeled nucleotides in the DNA containing sample to enable analysis of the base pairs present within the DNA. Many of the next-generation sequencing (NGS) technologies use a form of sequencing by synthesis (SBS), wherein modified nucleotides are used along with an enzyme to read the sequence of DNA templates in a controlled manner. In embodiments, sequencing includes a sequencing by synthesis event, where individual nucleotides are identified iteratively (e.g., incorporated and detected into a growing complementary strand), as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444 and 7,057,026. Once such a modified nucleotide has been incorporated into the growing polynucleotide chain complementary to the region of the template being sequenced, there is no free 3′—OH group available to direct further sequence extension and therefore the polymerase cannot add further nucleotides. Once the identity of the base incorporated into the growing chain has been determined, the 3′ reversible terminator may be removed to allow addition of the next successive nucleotide. In embodiments, the nucleic acid sequencing device utilizes the detection of four different nucleotides that include four different labels.

The term “nucleic acid sequencing device” means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ such as the G4™ sequencing platform, Illumina™, Inc. (e.g., HiSeq™, MiSeq™, NextSeq™, or NovaSeq™ systems), Life Technologies™ (e.g., ABI PRISM™, or SOLID™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri (carboxyethyl) phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light cmitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS).

The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

In embodiments, the systems and devices generate data that may be used to form an image. In embodiments, the image includes a 2D or 3D representation of the tissue. In some embodiments, one or more of the images include an image of other analytes, such as proteins in a biological sample. In some embodiments, an image is acquired using transmission light microscopy (e.g., bright field transmission light microscopy, dark field transmission light microscopy, oblique illumination transmission light microscopy, dispersion staining transmission light microscopy, phase contrast transmission light microscopy, differential interference contrast transmission light microscopy, emission imaging, etc.). In embodiments, the image is in any file format including but not limited to JPEG/JFIF, TIFF, Exif, PDF, EPS, GIF, BMP, PNG, PPM, PGM, PBM, PNM, WebP, HDR raster formats, HEIF, BAT, BPG, DEEP, DRW, ECW, FITS, FLIF, ICO, ILBM, IMG, PAM, PCX, PGF, JPEG XR, Layered Image File Format, PLBM, SGI, SID, CDS, CPT, PSD, PSP, XCF, PDN, CGM, SVG, PostScript, PCT, WMF, EMF, SWF, XAML, and/or RAW. In embodiments, the image is represented as an array (e.g., matrix) comprising a plurality of pixels, such that the location of each respective pixel in the plurality of pixels in the array (e.g., matrix) corresponds to its original location in the image. In some embodiments, an image is represented as a vector comprising a plurality of pixels, such that each respective pixel in the plurality of pixels in the vector comprises spatial information corresponding to its original location in the image.

In embodiments, microfluidic device includes a computing deveice, which is configured to provide an image for display via a display of the computing device. In embodiments, the image is provided for display via a GUI configured within the display of the computing device. In embodiments, the data processor receives an input identifying one or more modifications and/or one or more image analysis steps based on the provided image. For example, the display of the computing device can include a touchscreen display configured to receive a user input identifying a respective pattern of an image of the biological sample on the displayed image. In embodiments, the GUI can be configured to receive a user provided input identifying the modifications and/or one or more image analysis steps.

### EXAMPLES

Early biological experiments revealed proteins as the main agents of biological function. As such, proteins ultimately determine the phenotype of all organisms. Proteins do not function in isolation; instead, it is their interactions with one another and also with other molecules (e.g., DNA, RNA, hormones, carbohydrates) that mediate metabolic and signaling pathways, cellular processes, and organismal systems. The concept of “protein interaction” is generally used to describe the physical contact between proteins and their interacting partners and any subsequent downstream effects. Proteins typically interact in pairs to form dimers (e.g., reverse transcriptase), multi-protein complexes (e.g., the proteasome for molecular degradation or the nucleosome (NuRD) chromatin remodeling complex), or long chains (e.g., actin filaments in muscle fibers). The subunits creating the various complexes can be identical or heterogeneous (e.g., homodimers vs. heterodimers) and the duration of the interaction can be transient (e.g., proteins involved in signal transduction) or permanent (e.g., some ribosomal proteins). Historically, the main source of knowledge about protein interactions has come from biophysical methods, particularly from those based on deducing information based on structural information (e.g., X-ray crystallography, NMR spectroscopy, fluorescence, and/or atomic force microscopy) (see, Gonzalez MW and Kann MG. PLOS Comput. Biol. 2012; 8 (12): e1002819, which is incorporated herein by reference in its entirety). Biophysical methods can identify interacting partners, and also provide detailed information about the biochemical features of the interactions, such as binding mechanism and allosteric changes involved. Yet, since they are time- and resource-consuming, biophysical characterizations only permit the study of a few complexes at a time, typically without any spatial information about the cellular or tissue-specific localization of a protein complex.

Protein biomarker discovery enables identification of signatures with pathophysiological importance, bridging the gap between genomes and phenotypes. This type of data may have a profound impact on improving future healthcare, particularly with respect to precision medicine, but progress has been hampered by the lack of technologies that can provide reliable specificity, high throughput, sufficient precision, and high sensitivity. Expanding the knowledge of cellular protein interaction networks is vital to improve our understanding of several types of diseases, including cancer. Improved methods to study these interaction networks, especially in clinical settings, is therefore of great importance both for increasing the knowledge of the underlying disease mechanics, but also for finding new biomarkers for improved disease diagnostics and treatment response prediction. Another context where multiplexed detection of protein-protein interactions provides decisive importance is in the field of network pharmacology, where drugs are designed to act on several drug targets simultaneously. The rationale being that as cellular interaction networks are quite robust because of their underlying structure, to perturb these networks and to avoid escape mutations in malignancy, it may prove crucial to target several proteins simultaneously.

Mammalian cells are organized into different compartments that separate and facilitate physiological processes by providing specialized local environments and allowing different, otherwise incompatible biological processes to be carried out simultaneously. Proteins are targeted to these subcellular locations where they fulfill specialized, compartment-specific functions. Spatial proteomics aim to localize and quantify proteins within subcellular structures to provide three important biological insights. Firstly, spatial proteomics enables placing a protein in a specific location within the cell provides a hypothesis about what function the protein might have. For example, proteins localized to the mitochondria could have roles in energy production or apoptosis. Secondly, it can indicate a specific state of the cell or provide potential hypotheses about a new function of a protein if the protein is found in different subcellular locations simultaneously or upon perturbation. Thirdly, determining the localization of proteins is important to understand the functions of organelles and compartments. Most importantly, spatial proteomics of the non-perturbed state also provides a baseline for detecting aberrant localization of proteins, which is an important cause for a number of different human diseases (scc, Pankow S et al. Curr. Opin. Chem. Biol. 2019; 48:19-25).

Studies of the human proteome have begun to reveal a complex architecture, including single-cell variations, dynamic protein translocations, changing interaction networks, and proteins locating to multiple compartments. A typical human cell expresses more than 10,000 different proteins, spanning an abundance range of seven orders of magnitude. Current large-scale studies of the human spatial proteome suggest that it has a highly complex architecture that includes single-cell variation (in both protein level and localization), dynamic protein translocation, changing interaction networks and the localization of approximately half of all proteins to multiple compartments. The incorporation of global quantification data enables cellular model building and systems analyses that go beyond qualitative descriptions. Furthermore, several studies have successfully harnessed the power of global spatial proteomics to investigate diseases, including acute viral infection and liver disease, or to pinpoint the cellular defects that underlie monogenic disorders (see, e.g., Lundberg E and Borner GHH. Nat. Rev. Mol. Cell. Biol. 2019; 20 (5): 285-302).

Sensitive detection of protein interactions and post-translational modifications of native proteins is a challenge for research and diagnostic purposes. A method for this, which could be used in point-of-care devices and high-throughput screening, should be reliable, cost effective and robust. Existing in situ protcomic assays provide information on protein expression and cellular localization, typically with the use of fluorophore-labeled probes or enzyme-labeled oligonucleotides, while any associated sequencing information (e.g., sequencing of an antibody-conjugated oligonucleotide barcode) is obtained ex situ, therein losing any spatial proteomic information. Two approaches used for detecting protein interactions in situ include the proximity ligation assay (PLA) and the proximity extension assay (PEA). Proximity probes include protein binding domain, such as antibodies or aptamers. Examples of aptamer affinity probes may be found in, for example, Fredriksson S et al. Nat. Biotechnol. 2002; 20 (5): 473-477.

Proximity ligation assay (PLA) combines multiple recognition events with potent signal amplification. The method is based on pairs of proximity probes (that is, antibodies conjugated to strands of DNA) to detect the proteins of interest (see, e.g., Alam M. Curr. Protoc. Immunol. 2018; 123 (1): c58, which is incorporated herein by reference in its entirety). PLA assays have been commercialized, for example as Duolink® PLA technology from Sigma. Only on proximal binding of these probes can an amplifiable DNA strand be generated by ligation, which then is amplified by PCR. For localized detection, rolling circle amplification (RCA), an isothermal DNA amplification technique, may be used. RCA amplifies a circular template and generates long DNA strands that collapse into bundles of DNA. These bundles can be visualized by hybridizing fluorophore-labelled oligonucleotides to them and quantifying the number and intensity of dots by fluorescence microscopy, or by enzyme-labeled detection oligonucleotides, making it possible to detect single molecules in situ (see, e.g., Klaesson A et al. Sci. Rep. 2018; 8 (1): 5400, which is incorporated herein by reference in its entirety). By way of an additional example, Raykova et al. reported using an in situ PLA protocol to detect protein interactions between endogenous proteins in subcellular locations in fixed tissues and tissue sections by using antibody-DNA oligonucleotide conjugates to facilitate target capture and signal amplification (Raykova et al. Nat Commun. 2022 Aug. 13;13 (1): 4755). Similar to the aforementioned applications of PLA, the mode for detecting of protein interactions as described in Raykova's method is limited to fluorescence microscopy.

Proximity extension assay (PEA) typically utilizes two matched antibodies (e.g., two antibodies targeting the same protein) labelled with unique DNA oligonucleotides that simultaneously bind to a target protein in solution (for example, as commercialized by the Olink® PEA platform; for additional information on PEA see, e.g., International Patent Pub. Nos. WO 01/61037, WO 03/044231, WO 2004/094456, WO 2005/123963, WO 2006/137932, WO 2013/113699, and WO 2021/191442, each of which are incorporated herein by reference in their entirety). This brings the two antibodies into proximity, allowing their DNA oligonucleotides to hybridize, serving as template for a DNA polymerase-dependent extension step. This creates a double-stranded DNA “barcode” which is unique for the specific antigen and quantitatively proportional to the initial concentration of target protein. The hybridization and extension are immediately followed by PCR amplification. The resulting DNA amplicon can then be quantified either by qPCR, or by NGS-based approaches, depending on the specific protocol used. The exponential amplification properties of PCR are utilized in PEA to achieve a strong readout signal, providing assay sensitivity on par or better than traditional enzyme-linked immunosorbent assays (ELISAs). Importantly, this also means that extremely small sample volumes are needed to measure large numbers of proteins simultaneously, which is greatly beneficial when precious samples are in limited supply, such as in studies using human samples from clinical cohorts or biobank material (see, e.g., Lundberg M et al. Nucleic Acids Res. 2011; 39 (15): e102 and Assarsson E et al. PLOS ONE. 2014; 9 (4): e95129, each of which is incorporated herein by reference). A significant limitation of existing PLA and PEA methods for protein interaction detection, though, is that a priori knowledge of the protein targets of interest is required to select, for example, the antibodies of interest. These methods may therefore require additional validation of protein interactions, or complementary data from other experiments, such as mass spectrometry analysis of protein complexes, prior to performing.

There is a need for new methods that can provide information on more than isolated protein interaction events, such as the simultaneous detection of several interactions. Such methods can uncover protein interaction networks, aid in understanding protein complex architectures in tissue-specific contexts, and provide better diagnostics and treatment options. Existing in situ proteomic assays provide information on protein expression and cellular localization, while sequencing information is obtained ex situ. Beyond quantifying protein expression data, obtaining precise information about the identity and localization of protein complexes will support the identification of malignancies, monitor aberrant protein activity, and support the development of targeted treatments at the molecular level. Disclosed herein are solutions to these and other problems in the art.

The compositions and methods described herein provide sequence-level resolution of protein interactions while retaining spatial information. Additionally, these methods allow for de novo identification of protein interaction networks in an in situ context, providing significantly more information than existing proteomic methods which either require known targets to be used or which require sequencing of target antibody barcodes to be performed ex situ, losing any spatial information.

### Example 1. Components for Detecting De Novo Proximal Protein Complexes In Situ

The approach described herein utilizes a Universal Padlock Probe (PLP; also referred to herein as a circularizable probe, a gap-fill padlock probe, or a coupling oligonucleotide), a nonlimiting example of which is illustrated in FIG. 1A. In this particular example, the PLP includes, from 5′ to 3′, a first oligonucleotide tag binding sequence (A′; also referred to herein as a 5′ arm, 5′ arm sequence, or 5′ arm region, of the PLP), which is complementary to the first PLP binding sequence (A) of the first oligonucleotide tag (5), of the first probe (FIG. 1C, 3) shown in FIG. 1C, a linker region (1), which serves to link the 5′ and 3′ arms of the PLP such that circularization of the PLP would occur once the gap between the 5′ and 3′ arms is filled (as is shown in FIGS. 1D and 1E), and a second oligonucleotide tag binding sequence (D′; also referred to herein as a 3′ arm, 3′ arm sequence, or 3′ arm region, of the PLP), which is complementary to the second PLP binding sequence (D) of the second oligonucleotide tag (8), of the second probe (FIG. 1C, 6).

Embodiments of a bridge oligonucleotide, or bridge oligo, are described herein. The bridge oligonucleotide may include two hybridization sequences consisting of targeted priming regions that flank a linker region. In the nonlimiting example illustrated in FIG. 1B, the bridge oligo includes two hybridization sequences, C′ and B′, wherein each is capable of hybridizing to separate oligonucleotide tags (5 and 8), thus forming a bridge between the two oligo tags when the tags are in close proximity to each other due to colocalization of the protein targets of their attached binding molecules (4 and 7).

Embodiments of oligonucleotide-conjugated proximity probes are described herein. Nonlimiting examples of oligonucleotide-conjugated proximity probes are illustrated in FIGS. 1C and 2C, referred to in each as a first probe and a second probe. In FIG. 1C, the first probe includes a first specific binding molecule (4; e.g., an antibody, affimer, aptamer, etc.) linked to a first oligonucleotide tag (5). The first oligonucleotide tag includes, from 5′ to 3′, a first padlock probe (PLP) binding sequence (A), which is complementary to the 5′ arm sequence of the Universal PLP (A′), a first barcode sequence (UMI1; also referred to herein as a first unique molecular identifier), and a first bridge binding sequence (B), which is complementary to the 3′ end sequence of the Universal Bridge (B′). The second probe includes a second specific binding molecule (7; e.g., an antibody, affimer, aptamer, etc.) linked to a second oligonucleotide tag (8). The second oligonucleotide tag includes, from 5′ to 3′, a second bridge binding sequence (C), which is complementary to the 5′ end sequence of the Universal Bridge (C′), a second barcode sequence (UMI2; also referred to herein as a second unique molecular identifier), and a second PLP binding sequence (D), which is complementary to the 3′ arm sequence of the Universal PLP (D′). Since, in the particular example in FIG. 1C, the first and second specific binding molecules are attached to the first and second oligonucleotide tags at their 5′ ends, respectively, 3′ extension and potential displacement of the first and second oligonucleotide tags may be prevented via the inclusion of modifications (e.g., dideoxynucleotide or a reversibly-terminated nucleotide) at their respective 3′ ends. Alternatively, the first and second specific binding molecules may be attached to the first and second oligonucleotide tags at their 3′ ends, respectively, as is illustrated in FIG. 2C. As such, 3′ extension and potential displacement of the first and second oligonucleotide tags may be prevented as a result of the first and second specific binding molecules serving at 3′ extension blockers. When applied to a biological sample (e.g., a cell or tissue), the probes bind to their respective targets, either different proteins (FIG. 1D) or different regions on the same protein (FIG. 1E).

FIGS. 1D and 1E both illustrate how the Universal PLP serves here as a “coupling oligonucleotide” in that it anneals to the two different oligonucleotide tags due to their close proximity to each other. After the annealing of the Universal Bridge to the different oligonucleotide tags, the 3′ end of the PLP and the Universal Bridge molecule may be extended to generate a complement of the UMI sequences, and a ligase joins the adjacent ends to form a closed circle, as depicted in FIG. 3. The circle oligonucleotide may then be amplified and detected (e.g., detected via hybridization of fluorescently-labeled probes or via sequencing, as described herein). The term “coupling oligonucleotide”, or “coupling oligo”, as used herein, is not limited to PLPs, and can refer to any nucleic acid that can anneal to two or more separate oligonucleotides when the two or more separate oligonucleotides are in close proximity to each other.

The proximity probes described herein may be used to detect two or more proteins present in a complex in situ a nonlimiting example of which is illustrated in FIGS. 1D and 2D. Additionally, the same approach may be used to detect single proteins through the use of two proximity probes targeting the same protein, a nonlimiting example of which is illustrated in FIGS. 1E and 2E. In contrast to existing methods for profiling protein expression, the methods described herein allow for parallel sequencing-based detection in situ and spatial profiling, including de novo biomolecular interactions.

### Example 2. Spatial Detection of Binary Protein Complexes

The localization of proteins, their interacting partners, and their complexes are precisely tuned to enable correct function of the proteins within their subcellular niches and tissues. Aberrant relocation and/or mislocalization of the proteins is considered to underlie the development of a variety of diseases (see, e.g., Breckels et al. Cell Chem Biol. 2024 Sep. 19;31 (9): 1665-1687, which is incorporated herein by reference in its entirety). Current single-cell proteomics methods suffer from low number of proteins analyzed in a study. Methods for high throughput, spatial detection of protein complexes in situ are greatly needed.

Described herein are compositions and methods used for spatial detection of binary protein complexes in situ. Prior to applying methods for spatial detection of binary protein complexes, the cell or tissue may be manipulated prior to immobilizing the cell or tissue onto a solid support using known techniques in the art (see, e.g., PCT Publication WO2023076832A1). Tissue sections may be manipulated using methods and techniques known in the art to for in situ proteomics workflows (see, e.g., U.S. Pat. No. 11,891,656, which is incorporated herein by reference in its entirety). Capture and spatial detection of a protein in a protein complex may performed using a protein-specific binding agent, such as first proximity probe (i.e., a first probe) and second proximity probe (i.e., a second probe) as described herein. In embodiments, a plurality of pairs of first probes and second probes are used to evaluate a plurality of distinct protein complexes in a tissue section simultaneously. In embodiments, the tissue is contacted with one, two, three, four, five, or more distinct pairs of first probes and second probes.

Embodiments for methods of spatial detection of binary protein complexes are described herein, nonlimiting examples of which are illustrated in FIGS. 1D and 2D. FIG. 1D illustrates an embodiment of a method described herein for spatial detection of protein interactions using the proximity probes (e.g., oligonucleotide-conjugated antibodies) described herein. In embodiments, the first probe (i.e., the first proximity probe) is attached to a first oligonucleotide including a first barcode sequence capable of hybridizing with a complementary sequence on the coupling oligonucleotide, and the second probe (i.e., the second proximity probe) is attached to a second oligonucleotide including a second barcode sequence capable of hybridizing with a complementary sequence on the coupling oligonucleotide. Shown here is a protein complex (11), as found in a cell, wherein the complex includes a Protein 1 bound directly to a Protein 2. A first proximity probe (3; FIG. 1C) binds to Protein 1, forming a protein-probe complex (9), and will thus be proximal to a second proximity probe (6; FIG. 1C) which forms a protein-probe complex with Protein 2. As such, the first and second probe tag oligonucleotides (5 and 8 respectively; FIG. 1C) are close enough to each other such that they can both hybridize to the couple oligonucleotide (e.g., a single Universal PLP as shown in FIG. 1A) to facilitate the formation of a linked complex. In embodiments, the linked complex incudes the protein-probe complex and the coupling oligonucleotide, wherein the protein-probe complex includes the first probe bound to the first protein target (shown as Protein 1 in FIGS. 1C and 2C) and the second probe bound to the second protein target (shown as Protein 2 in FIGS. 1C and 2C). In embodiments, the coupling oligonucleotide is hybridized to the first oligonucleotide (harboring UMI-1) of the first probe and second oligonucleotide (harboring UMI-2) of the second probe. Following the formation of the linked complex, a bridge oligonucleotide as described herein (e.g., a single bridging oligo as shown in FIG. 1B) may be added to hybridize a first sequence of the bridge oligonucleotide to the first oligonucleotide of the first probe and to hybridize a second sequence of the bridge oligonucleotide to the second oligonucleotide of the second probe to facilitate the formation of a linked-bridge complex. Following the formation of the linked-bridge complex, the coupling oligonucleotide could be extended with a polymerase to generate the complement of the second barcode sequence (e.g., shown as UMI-2 in FIGS. 1D and 2D); the bridge oligonucleotide could be extended with a polymerase to generate the complement of the first barcode sequence (e.g., shown as UMI-1 in FIGS. 1D and 2D); and the resultant extension products may be ligated using a ligase to generate a circular construct made up of the four hybridized nucleic acids (10). In embodiments, the circular oligonucleotide is amplified prior to detection to boost its signal for detection. In embodiments, the mode of detection is by sequencing-by-synthesis, where the sequence of first barcode and second barcode (or complement thereof) are detected and used to associate and identify the proteins captured from the binary protein complex in the tissue section following bioinformatic analyses.

In this particular nonlimiting example, the first probe tag oligonucleotide has a first unique barcode sequence (UMI-1) to be used for the purpose of identifying the protein that the probe specifically binds to (Protein 1). This first barcode sequence is flanked by a 5′ sequence (A) which hybridizes with the 5′ arm region of the Universal PLP (A′), and a 3′ region (B) that hybridizes with the 3′ tag binding region of the bridging oligo (B′). The second probe tag oligonucleotide has a second unique barcode sequence (UMI-2) to be used for the purpose of identifying the protein that the probe specifically binds to (Protein 2). This second barcode sequence is flanked by a 5′ sequence (C) which hybridizes with the 5′ tag binding region of the bridging oligo (C′), and a 3′ region (D) that hybridizes with the 3′ arm region of the Universal PLP (D′). Upon the annealing of the PLP with the two target oligos, and the annealing of the two target oligos with the bridging primer, as is illustrated in FIG. 1D (10), the 3′ end of the hybridized bridging oligo is extended (e.g., using a non-strand displacing polymerase), to generate a sequence that is complementary to the first barcode sequence, shown as UMI-l′ in FIG. 3. Additionally, the 3′ end of the hybridized PLP is extended (e.g., using a non-strand displacing polymerase), to generate a sequence that is complementary to the second barcode sequence, shown as UMI-2′ in FIG. 3. Following extension, the 3′ end of the newly formed first complementary barcode sequence (UMI-1′) is ligated to the 5′ end of the PLP, and the 3′ end of the newly formed second complementary barcode sequence (UMI-2′) is ligated to the 5′ end of the bridging oligo using, for example, a ligase, thereby generating a circularized probe, an example of which is illustrated in FIG. 3.

FIGS. 4A and 4B illustrate the steps of amplifying the circularized probe (e.g., by rolling circle amplification using a processive strand-displacing polymerase (13), thereby generating a concatemer of amplification products (FIG. 4B). In this particular nonlimiting example, FIG. 4A illustrates a circularized probe (e.g., of FIG. 3), primed with an amplification primer (12) and extended with a strand-displacing polymerase (13) to generate a concatemer containing multiple copies of the target nucleic acid sequence (14; FIG. 4B).

In embodiments, the padlock probe further includes an amplification priming site (i.e., a nucleic acid sequence complementary to an amplification primer) and a distinct sequencing priming site (i.e., a nucleic acid sequence complementary to a sequencing primer). Alternatively, in embodiments, the padlock probe further includes an amplification priming site and a sequencing priming site that are the same, are partially overlapping, or in which one is internal to the other.

The amplification products are then detected, for example, by hybridizing a sequencing primer to a plurality of sequencing primer binding sequences (illustrated as PBS1 and PBS2 in FIGS. 4C and 4D) on the amplification product, incorporating labeled nucleotides with a polymerase, and detecting the labels to identify the incorporated bases. The amplification products may also be detected using fluorescently labeled probes. Additional alternative modes of detection are contemplated herein, for example FISH, SBB, and the like. In embodiments, the primer binding sequence is complementary to a fluorescent in situ hybridization (FISH) probe. FISH probes may be custom designed using known techniques in the art, see for example Gelali, E., et al. Nat Commun 10, 1636 (2019). Additional methods based on single molecule fluorescence in situ hybridization may also be used for detection. These include MERFISH (Multiplexed Error-Robust Fluorescence In Situ Hybridization), STARmap (Spatially-resolved Transcript Amplicon Readout mapping), FISSEQ, BaristaSeq, seq-FISH (Sequential Fluorescence In Situ Hybridization) and others (see for example Chen, K. H., et al. (2015). Science, 348 (6233), aaa6090; Wang, G., Moffitt, J. R. & Zhuang, X. Sci Rep. 2018; 8, 4847; Wang X. et al.; Science, 2018; 27, Vol 361, Issue 6400, caat5691; Cai, M. Dissertation, (2019) UC San Diego. ProQuest ID: Cai_ucsd_0033D_18822; and Sansone, A. Nat Methods 16, 458; 2019).

Provided are embodiments for spatial detection of multiple protein interactions, in parallel, using the proximity probes (e.g., oligonucleotide-conjugated antibodies) described herein. A nonlimiting example is illustrated in FIG. 5. Shown here are two protein complexes in a cell, wherein the first complex (15) includes Protein A bound to Protein B, while the second complex (16) includes Protein C bound to Protein B. A first proximity probe (17) is bound to Protein A and is proximal to a second proximity probe (18), which is bound to Protein B, such that the first and second probe tag oligonucleotides can hybridize to the PLP and bridging oligos as described within. In this particular case, although the Protein B barcode is the same in both complexes, the interaction of Protein B with Protein A is distinguished from the interaction of Protein B with Protein C due to the unique barcodes formed when the Protein B and Protein A barcodes, and Protein B and Protein C barcodes, are combined as can be seen at the bottom of FIG. 5.

The methods described herein provide a novel way to obtain a comprehensive in situ view of protein interactions without the need to use laborious and expensive techniques such as mass spectrometry. The barcoded proximity probes can be scaled up or down to target numerous protein complexes in a sample. The methods provide unique insight into the spatial localization of protein complexes, for example, how protein complex components may vary depending on the tissue or cell under investigation, or under disease conditions.

### Example 3. Spatial Detection of Cellular Protein Interactomes

Spatial proteomics aims to localize and quantify proteins within subcellular structures to provide three important biological insights. Firstly, spatial proteomics enable placing a protein in a specific location within the cell provides a hypothesis about what function the protein might have. For example, proteins localized to the mitochondria could have roles in energy production or apoptosis. Secondly, it can indicate a specific state of the cell or provide potential hypotheses about a new function of a protein if the protein is found in different subcellular locations simultaneously or upon perturbation. Thirdly, determining the localization of proteins is important to understand the functions of organelles and compartments. Most importantly, spatial proteomics of the non-perturbed state also provides a baseline for detecting aberrant localization of proteins, which is an important cause for a number of different human diseases. Because spatial proteomics typically requires the enrichment of proteins prior to identification, results are fundamentally limited with regards to several basic aspects in subcellular biology of proteins (see, Pankow S et al. Curr. Opin. Chem. Biol. 2019; 48:19-25). Differences in protein abundance and localization can be dynamic and observed across macro-, meso-, and microscopic scales of tissues and cells. Existing methods for detecting cellular proteomes involve complex and expensive workflows, such as performing tandem-affinity purification of affinity tag-labeled proteins followed by mass spectrometry (see, e.g., Adelmant G et al. Curr. Protoc. Protein Sci. 2019; 96 (1): e84, which is incorporated herein by reference in its entirety). New methods are needed to assess protein interactomes in situ, retaining spatial information while providing high-resolution identification of novel protein complexes.

In situ sequencing involves tissue and/or cellular extraction, combined with the fixation and permeabilization of cells, followed by amplification of the target nucleic acid fragments for sequencing. Briefly, cells and their surrounding milieu are attached to a substrate surface, fixed, and permeabilized using known methods.

FIGS. 6A-6C illustrate further embodiments of the methods described herein for detecting a protein complex in situ using the proximity probes (e.g., oligonucleotide-conjugated antibodies) described herein.

FIG. 6A illustrates a protein complex in a cell including Protein A, Protein B, Protein C and Protein D. In this illustrated complex, Protein A is bound to Protein B, Protein C is bound to Protein D, and Proteins B and D are bound to each other. While Proteins A and C are not directly bound to each other, their close proximity to each other, as members of the Protein A-B-C-D Complex can be detected with a first proximity probe (First Probe), which binds to Protein A, and a second proximity probe (Second Probe), which binds to Protein C, using the methods described within.

FIG. 6B illustrates a protein complex in a cell including Protein A, Protein B, and Protein C. In this illustrated complex, Protein A and Protein C are both bound the Protein B, but are not directly bound to each other. A first proximity probe (First Probe) is bound to Protein A, and a second proximity probe (Second Probe) is bound to Protein C. As described above, while Proteins A and C are not directly bound to each other, their close proximity to each other, as members of the Protein A-B-C Complex can be detected with a first proximity probe (First Probe), which binds to Protein A, and a second proximity probe (Second Probe), which binds to Protein C, using the methods described within.

FIG. 6C shows how these methods can be used to determine the proximity of proteins with other types of compounds as well (i.e., carbohydrates). As such the posttranslational modifications of these proteins (i.e., glycosylation), and the correlation the modifications may have with protein-protein associations and localizations, may be determined.

The methods described herein provide a novel way to obtain a comprehensive in situ view of protein interactions without the need to perform ex situ sequencing or use laborious and expensive techniques such as mass spectrometry. Cellular protein interactomes are able to be identified in their native context without the need to introduce exogenously expressed proteins with affinity tags (e.g., FLAG and/or HA peptide epitopes). The barcoded proximity probes described herein can be scaled up or down to multiplex targeting of numerous protein complexes in a sample. These methods provide unique insight into the spatial localization of protein complexes, for example, how protein complex components may vary depending on the tissue or cell under investigation, or under disease conditions.

Although the Examples described supra and herein outline in situ sequencing approaches, it will be appreciated that the methods may be modified such that the barcode-containing oligonucleotides are removed from the cell (e.g., the cell is harvested and the oligonucleotides purified or captured using affinity capture) and then sequenced on an instrument, ex situ. For example, the circularized PLP with the two or more barcode sequences can be removed and sequenced outside of the cell using standard sequencing approaches (e.g., sequenced on a Singular Genomics G4X™ Sequencing Platform).

Alternatively, the circularized padlock probe including the complementary sequences of the two or more barcode sequences is purified and/or capture from the cell, and sequenced ex situ. The padlock probe may be circularized in the cell or after removal from the cell, and may be amplified prior to sequencing, wherein the amplification occurred in the cell or the amplification is performed outside of the cell prior to sequencing.

### Example 4: Characterizing Protein-Protein Interactions in Disease States

Protein interaction networks (e.g., network maps that annotate protein interactions with single or multiple binding partners in a given biological context) are useful resources in the abstraction of basic science knowledge and in the development of biomedical applications. By studying protein interaction networks, we can learn about the evolution of individual proteins and about the different systems in which they are involved. Due to their central role in biological function, protein interactions also control the mechanisms leading to healthy and diseased states in organisms. Diseases are often caused by mutations affecting the binding interface or leading to biochemically dysfunctional allosteric changes in proteins. Therefore, protein interaction networks can elucidate the molecular basis of disease, which in turn can inform methods for prevention, diagnosis, and treatment (see, Gonzalez MW and Kann MG. PLOS Comput. Biol. 2012; 8 (12): e1002819). As protein interactions mediate the healthy states in all biological processes, it follows that they should be key targets of the molecular-based studies of biological diseased states.

Protein interactions are known to be disrupted or altered in several human disease states. For example, pathogen-host interactions play a key role in bacterial and viral infections. The Human papillomavirus, upon infection, expression two viral genes, E6 and E7, which interaction with negative cell regulatory proteins to target them for degradation, allowing the virus to bypass the immune system (see, Scheffner M et al. Semin. Cancer Biol. 2003; 13:59-67). In other diseases, such as Huntington's disease, cystic fibrosis, and Alzheimer's disease, mutations may lead to unwanted protein interactions (e.g., mutations that lead to toxic misfolded proteins) that can alter homeostatic protein networks and lead to disease. In the case of Huntington's disease, glutamine expansion in the Huntingtin protein leads to alternate conformational states that induce toxic protein interactions (see, Duennwald ML et al. Proc. Natl. Acad. Sci. USA. 2006; 103 (29): 11051-6). Further, oncogenic protein-protein interactions have been found to have a high correlation with patient survival and drug resistance/sensitivity, with the recent observation that somatic missense mutations are enriched at the interfaces of protein-protein interactions in cancer (see, Cheng F et al. Nat. Genet. 2021; 53:342-353, which in incorporated herein by reference in its entirety).

Amino acid substitutions in vinculin (VCL), located at the interaction interface between VCL and fragile X mental retardation syndrome-related protein 1 (FXFR1) are significantly correlated with resistance to incorafenib, an FDA-approved BRAF inhibitor for the treatment of melanoma, compared to patients without VCL-FXFR 1-perturbing mutations (see, Koelblinger P et al. Curr. Opin. Oncol. 2018; 30:125-133). The methods described herein provide a novel in situ proteomic approach for obtaining detailed protein-protein interaction information from diseased tissue, such as tumor tissue, for example, in a patient undergoing treatment for melanoma. Briefly, a tumor tissue section is attached to a substrate surface, fixed, and permeabilized according to known methods in the art. Tissue sections may be manipulated using methods and techniques known in the art to for in situ proteomics workflows (see, e.g., U.S. Pat. No. 11,891,656, which is incorporated herein by reference in its entirety). Capture and spatial detection of a protein-protein interaction may performed using a protein-specific binding agent, such as first proximity probe (i.e., a first probe) and second proximity probe (i.e., a second probe) as described herein. In embodiments, a plurality of pairs of first probes and second probes are used to evaluate a plurality of disease-relevant protein-protein interactions in a tissue section simultaneously. In embodiments, the tissue is contacted with one, two, three, four, five, or more distinct pairs of first probes and second probes. The methods described in Example 2 are then performed, using a first proximal probe (e.g., a first probe described herein) specific for VCL and a second proximal probe (e.g., a second probe described herein) for FXFR1. In embodiments, the first probe specific for VCL is attached to a first oligonucleotide including a first barcode sequence capable of hybridizing with a complementary sequence on the coupling oligonucleotide, and the second probe specific for FXFR1 is attached to a second oligonucleotide including a second barcode sequence capable of hybridizing with a complementary sequence on the coupling oligonucleotide. Following the binding of the first probe and second probe to VCL and FXFR1, respectively, a coupling oligonucleotide as described herein may be added to the tumor tissue section to facilitate the formation of the linked complex including the protein-probe complex and the coupling oligonucleotide, wherein the protein-probe complex includes the first probe bound to VCL and the second probe bound to FXFR1 and wherein the coupling oligonucleotide is hybridized to the first oligonucleotide and second oligonucleotide. Following the formation of the linked complex, a bridge oligonucleotide as described herein may be added to hybridize a first sequence of the bridge oligonucleotide to the first oligonucleotide of the first probe and to hybridize a second sequence of the bridge oligonucleotide to the second oligonucleotide of the second probe, which facilitates the formation of a linked-bridge complex. Following extension and removal (e.g., digestion) of the second probe oligonucleotide, a circularizable probe is hybridized to the first probe oligonucleotide, extended, circularized, and amplified, as illustrated in FIGS. 1D and 1E. This extension product is then primed with a sequencing primer and subjected to sequencing processes as described herein, thereby providing a high-resolution view of molecular features that can be combined with additional histological findings for clinical decision-making.

### NUMBERED EMBODIMENTS

Embodiment 1. A method of forming a circular oligonucleotide, the method including: (i) contacting a protein-probe complex with a coupling oligonucleotide, wherein the protein-probe complex includes a protein bound to a first probe and a second probe, the first probe attached to a first oligonucleotide including a first barcode sequence and the second probe attached to a second oligonucleotide including a second barcode sequence, and hybridizing a first sequence of the coupling oligonucleotide to the first oligonucleotide and hybridizing a second sequence of the coupling oligonucleotide to the second oligonucleotide, thereby forming a linked complex including the protein-probe complex and the coupling oligonucleotide; (ii) contacting the linked complex with a bridge oligonucleotide and hybridizing a first sequence of the bridge oligonucleotide to the first oligonucleotide and hybridizing a second sequence of the bridge oligonucleotide to the second oligonucleotide, thereby forming a linked-bridge complex; and (iii) extending the second sequence of the coupling oligonucleotide with a polymerase to form a complement of the second barcode sequence; extending the second sequence of the bridge oligonucleotide with a polymerase to form a complement of the first barcode sequence; and ligating the complement of the second barcode sequence to the first sequence of the bridge oligonucleotide and ligating the complement of the first barcode sequence to form the circular oligonucleotide.

Embodiment 2. The method of Embodiment 1, prior to step (i), forming the protein-probe complex by binding the first probe to the protein, wherein the first probe includes a first specific binding reagent attached to the first oligonucleotide, and binding the second probe to the protein, wherein the second probe includes a second specific binding reagent attached to the second oligonucleotide.

Embodiment 3. The method of Embodiment 2, wherein the second probe binds to a carbohydrate attached to the protein.

Embodiment 4. A method of forming a circular oligonucleotide, the method including: (i) binding a first probe to a first protein and binding a second probe to a second protein, wherein the first probe includes a specific binding reagent attached to a first oligonucleotide including a first barcode sequence, and the second probe includes a specific binding reagent attached to a second oligonucleotide including a second barcode sequence, thereby forming a protein-probe complex; (ii) contacting the protein-probe complex with a coupling oligonucleotide and hybridizing a first sequence of the coupling oligonucleotide to the first oligonucleotide and hybridizing a second sequence of the coupling oligonucleotide to the second oligonucleotide, thereby forming a linked complex including the protein-probe complex and the coupling oligonucleotide; (iii) contacting the linked complex with a bridge oligonucleotide and hybridizing a first sequence of the bridge oligonucleotide to the first oligonucleotide and hybridizing a second sequence of the bridge oligonucleotide to the second oligonucleotide, thereby forming a linked-bridge complex; and (iv) extending the second sequence of the coupling oligonucleotide with a polymerase to form a complement of the second barcode sequence; extending the second sequence of the bridge oligonucleotide with a polymerase to form a complement of the first barcode sequence; and ligating the complement of the second barcode sequence to the first sequence of the bridge oligonucleotide and ligating the complement of the first barcode sequence to form the circular oligonucleotide.

Embodiment 5. The method of Embodiment 4, wherein the first protein and the second protein are both bound to a third protein.

Embodiment 6. The method of Embodiment 4, wherein the first protein is bound to a third protein and the second protein is bound to a fourth protein.

Embodiment 7. The method of any one of Embodiments 1 to 6, further including detecting the first barcode sequence and the second barcode sequence.

Embodiment 8. The method of Embodiment 7, wherein detecting includes sequencing.

Embodiment 9. The method of Embodiment 8, wherein sequencing includes extending a sequencing primer by incorporating a labeled nucleotide, or labeled nucleotide analogue and detecting the label to generate a signal for each incorporated nucleotide or nucleotide analogue.

Embodiment 10. The method of any one of Embodiments 1 to 9, further including amplifying the circular oligonucleotide.

Embodiment 11. The method of Embodiment 10, wherein amplifying includes hybridizing an amplification primer to the circular oligonucleotide and extending the primer with a strand-displacing polymerase to generate an amplification product including multiple complements of the circular oligonucleotide.

Embodiment 12. The method of Embodiment 10, wherein amplifying includes hybridizing an amplification primer to the circular oligonucleotide and extending the primer with a non-strand-displacing polymerase to generate an amplification product.

Embodiment 13. The method of any one of Embodiments 1 to 12, further including sequencing the amplification product.

Embodiment 14. The method of any one of Embodiments 1 to 13, wherein the first specific binding reagent is an antibody, single-chain Fv fragment (scFv), antibody fragment-antigen binding (Fab), lectin, affimer, or an aptamer and wherein the second specific binding reagent is an antibody, single-chain Fv fragment (scFv), antibody fragment-antigen binding (Fab), lectin, affimer, or an aptamer.

Embodiment 15. The method of any one of Embodiments 1 to 14, wherein the first specific binding reagent is an antibody and the second specific binding reagent is an antibody.

Embodiment 16. The method of any one of Embodiments 1 to 15, wherein the first oligonucleotide is attached to the first probe via a linker, and wherein the second oligonucleotide is attached to the second probe via a linker.

Embodiment 17. The method of any one of Embodiments 1 to 16, wherein the first oligonucleotide includes, from 5′ to 3′, a first coupling sequence, a first barcode sequence, and a first bridge sequence; and wherein the second oligonucleotide includes, from 5′ to 3′, a second bridge sequence, a second barcode sequence, and a second coupling sequence.

Embodiment 18. The method of any one of Embodiments 1 to 17, wherein the coupling oligonucleotide includes a primer binding sequence.

Embodiment 19. The method of any one of Embodiments 1 to 18, wherein the coupling oligonucleotide includes, from 5′ to 3′, a first sequence complementary to the first coupling sequence of the first oligonucleotide, a spacer sequence, and a second sequence complementary to the second coupling sequence of the second oligonucleotide.

Embodiment 20. The method of any one of Embodiments 1 to 19, wherein the coupling oligonucleotide includes 50 to 500 nucleotides.

Embodiment 21. The method of any one of Embodiments 1 to 20, wherein the bridge oligonucleotide includes, from 3′ to 5′, a first sequence complementary to a first bridge sequence of the first oligonucleotide, a spacer sequence, and a second sequence complementary to a second bridge sequence of the second oligonucleotide.

Embodiment 22. The method of any one of Embodiments 1 to 21, wherein the bridge oligonucleotide includes 20 to 100 nucleotides.

Embodiment 23. The method of any one of Embodiments 1 to 22, wherein the protein-probe complex is in a cell or tissue.

Embodiment 24. The method of Embodiment 23, wherein the cell or

tissue is fixed to a solid support.

Embodiment 25. The method of Embodiment 24, wherein the solid support includes a functionalized glass surface or a functionalized plastic surface.

What is claimed is:

1. A method of sequencing two barcode sequences, said method comprising:
a) binding a first probe to a first target molecule and binding a second probe to a second target molecule, first probe attached to a first oligonucleotide comprising a first barcode sequence and said second probe attached to a second oligonucleotide comprising a second barcode sequence;
b) forming a circular oligonucleotide comprising complements of the first barcode sequence and the second barcode sequence;
c) amplifying the circular oligonucleotide to form amplification products comprising the first barcode sequence and the second barcode sequence; and
d) hybridizing a sequencing primer to an amplification product and sequencing the first barcode sequence and the second barcode sequence.

2. The method of claim 1, wherein the first target molecule is a protein, and the second target molecule is a carbohydrate.

3. The method of claim 1, wherein the first target molecule is a protein, and the second target molecule is a protein.

4. The method of claim 3, wherein the first protein and the second protein are each in or on a cell.

5. The method of claim 3, wherein the first protein and the second protein are each in a tissue.

6. The method of claim 1, wherein forming the circular oligonucleotide comprises hybridizing a first sequence of a coupling oligonucleotide to the first oligonucleotide and hybridizing a second sequence of the coupling oligonucleotide to the second oligonucleotide, thereby forming a linked complex; hybridizing a first sequence of a bridge oligonucleotide to the first oligonucleotide and hybridizing a second sequence of the bridge oligonucleotide to the second oligonucleotide, thereby forming a linked-bridge complex; and extending the second sequence of the coupling oligonucleotide with a polymerase to form a complement of the second barcode sequence; extending the second sequence of the bridge oligonucleotide with a polymerase to form a complement of the first barcode sequence; and ligating the complement of the second barcode sequence to the first sequence of the bridge oligonucleotide and ligating the complement of the first barcode sequence to form the circular oligonucleotide.

7. The method of claim 1, wherein the first probe is an antibody and the second probe is an antibody.

8. The method of claim 1, further comprising detecting the first target molecule and the second target molecule based on the identity of the barcode sequences.

9. The method of claim 1, wherein said first oligonucleotide comprises, from 5′ to 3′, a first coupling sequence, a first barcode sequence, and a first bridge sequence; and wherein said second oligonucleotide comprises, from 5′ to 3′, a second bridge sequence, a second barcode sequence, and a second coupling sequence.

10. The method of claim 6, wherein said coupling oligonucleotide comprises a primer binding sequence.

11. The method of claim 6, wherein said coupling oligonucleotide comprises, from 5′ to 3′, a first sequence complementary to the first coupling sequence of the first oligonucleotide, a spacer sequence, and a second sequence complementary to the second coupling sequence of the second oligonucleotide.

12. The method of claim 6, wherein said coupling oligonucleotide comprises 50 to 500 nucleotides.

13. The method of claim 6, wherein said bridge oligonucleotide comprises, from 3′ to 5′, a first sequence complementary to a first bridge sequence of the first oligonucleotide, a spacer sequence, and a second sequence complementary to a second bridge sequence of the second oligonucleotide.

14. The method of claim 6, wherein said bridge oligonucleotide comprises 20 to 100 nucleotides.

15. The method of claim 4, further comprising imaging the cell and determining a location of the first target molecule and the second target molecule.

16. The method of claim 1, wherein sequencing comprising monitoring sequential incorporation of modified nucleotides, wherein the modified nucleotides comprise a label and a reversible terminator, wherein the reversible terminator is removed prior to incorporation of a subsequent modified nucleotide.

17. The method of claim 5, wherein a)-d) are performed in situ.

18. A microfluidic device comprising a solid support, wherein said solid support comprises a cell or tissue comprising:
a first probe bound to a first target molecule and a second probe bound to a second target molecule, wherein the first probe is attached to a first oligonucleotide comprising a first barcode sequence and said second probe attached to a second oligonucleotide comprising a second barcode sequence;
a coupling oligonucleotide hybridized to the first oligonucleotide and to the second oligonucleotide; and
a bridge oligonucleotide hybridized to the first oligonucleotide and to the second oligonucleotide.

19. The microfluidic device of claim 18, wherein the first target molecule is a protein.

20. The microfluidic device of claim 18, wherein the tissue is a colon, breast tissue, lung, lymph, or bone tissue.
